(19) United States
(12) Patent Application Publication
(10) Pub. No.: US 2026/0358287 A1
(43) Pub. Date: Nov. 12, 2026

(54) ANTIMICROBIAL COATING FOR MEDICAL DEVICES WITH HIGH ELECTROCONDUCTIVITY AND BIOCOMPATIBILITY

(71) Applicant: PRESIDENT AND FELLOWS OF HARVARD COLLEGE, Cambridge, MA (US)

(72) Inventors: Sofia Wareham MATHIASSEN, Cambridge, MA (US); Pawan JOLLY, Cambridge, MA (US); Donald E. INGBER, Cambridge, MA (US)

(73) Assignee: PRESIDENT AND FELLOWS OF HARVARD COLLEGE, Cambridge, MA (US)

(21) Appl. No.: 19/130,319

(22) PCT Filed: Nov. 16, 2023
(86) PCT No.: PCT/US2023/079982

§ 371 Date: May 15, 2025

Related U.S. Application Data
(60) Provisional application No. 63/425,844, filed on Nov. 16, 2022.

(51) Int. Cl. G01N 27/327; A01N 59/16; A01N 63/50; A01P 1/00; A61K 31/352; A61K 31/439; A61K 31/573; A61K 31/58; C09D 5/16; C09D 5/24; G01N 27/414; G01N 33/543; G01N 33/58

(52) U.S. Cl. CPC G01N 27/3276; A01N 59/16; A01N 63/50; A01P 1/00; A61K 31/352; A61K 31/439; A61K 31/573; A61K 31/58; C09D 5/1662; C09D 5/24; G01N 27/4145; G01N 33/5438; G01N 33/581

(57) ABSTRACT

The disclosure relates generally to compositions and methods for making anti-fouling and electrically responsive coatings and electrodes, organic field effect transistors and organic sensors including said coatings, and uses thereof. These coatings protect conducting and transducer surfaces from unwanted interactions that impede or diminish their intended function, further exhibiting antimicrobial activity and preventing adverse physiological responses resulting from implantation or insertion in tissue.

### CROSS-REFERENCE TO RELATED APPLICATIONS

This application claims benefit under 35 U.S.C. § 119(e) of U.S. Provisional Application No. 63/425,844, filed Nov. 16, 2022, the contents of which are incorporated herein by reference in their entireties.

### TECHNICAL FIELD

The disclosure relates generally to compositions and methods for making anti-fouling and electrically responsive coatings and electrodes, organic field effect transistors and organic sensors including said coatings, and uses thereof.

### BACKGROUND

In recent years there has been rapid development of point-of-care (POC) devices which can be used by patients at home or medical professionals in a clinic for rapid detection or diagnosis without the need to deliver samples for pathological analysis. Electrochemical sensors and transistors such as organic thin film transistors are ideal for POC devices because of their compatibility with biological fluid and potential to interface with electronics without requiring further analytical equipment to analyze the signal.

One of the major obstacles in the development of electrochemical sensors, such as those in POC devices, is the rapid accumulation of proteins from biological fluids on the conductive surfaces of the sensor. While selectivity is an inherent property of the capture agent's affinity for its target, non-specific interactions can considerably decrease the sensitivity of an assay and result in false positives. This weakens the sensor signal and can deactivate the sensor. One solution has been to embed an enzyme into a porous material that the biological fluid can permeate. This approach is not suitable for all assays, for example, immunological assays. Furthermore, inflammatory responses may arise from the implantation process, the physiological mismatch between the mechanical moduli of the electrode and surrounding tissue, and other immunogenic implant constituents/surface characteristics. In addition, the development of a foreign body response (FBR) may lead to fibrous capsule formation surrounding the device, further compromising sensor functionality.

There is a need for point-of-care diagnostic devices conducting an assay in a biological fluid contacting the surface of a sensor or transistor. Preferably, the device minimizes the loss of signal to the sensor or transistor electrode.

Molecular blockers of varying molecular weight, including Bovine Serum Albumin (BSA), casein, pluronic acid, and poly(ethylene glycol) polymers (PEG) among others, have been used to limit non-specific binding interactions that may occur at surfaces and/or in solution. For example, the surface of microtiter plates used in enzyme linked sandwich immunoassays (ELISAs) are typically blocked with BSA to reduce non-specific adsorption of proteins at their surface and BSA is also usually added to the buffers used during the assay.

For assays based on a final optical readout (e.g. absorbance, fluorescence, chemiluminescence or electrochemiluminescence), the blockers do not interfere with the final measurement. This is because the assay chemistry and measurements are fully decoupled. The assay is carried out on a surface (e.g. plates, microbeads and nanoparticles) whereas the final measurement is performed using an external transducer. For example, in fluorescence based assays, light of a predetermined wavelength is shined on a surface bearing the capture agent and the light emitted is quantified by a photodiode or CCD sensor (i.e., the transducer). In the forgoing example, the surface where the molecular interaction takes place acts as a passive support and does not contribute to the measurement.

A more challenging situation is presented when an electrochemical read out is desired since the assay is carried out on the transducer surface. The capture agent is typically immobilized at the surface of the transducer using strategies that should maximize its density and orientation, prevent non-specific interactions, and at the same time, preserve the ability of the electrode to record electrochemical signals with high sensitivity. Accordingly, it is imperative to the development of these electrochemical sensors that its surface resists biofouling, the aggregation of biomolecules on the surface, and that it maintains its initial physical and/or chemical properties (e.g. conductivity).

One of the problems encountered in providing these anti-fouling coatings is deposition and attachment on various substrates. For example, gold is a good electrode coating substrate that has a high affinity to proteins due to thiol-gold interactions. However other surfaces can be more challenging.

Furthermore, the standard process to functionalize transducers with an anti-fouling coating is to drop cast a composition on the surface of the transducer and incubating overnight. This step adds a day to the process and thus lengthens the time to make the final sensors. Integration into a multi-step on continuous manufacturing process may be frustrated, since the coating step may constitute a bottleneck to the entire process.

There is therefore a need for coatings and methods for preparing these coating on various electrical transducers that can accommodate capture agents, prevent non-specific interaction, preserve the ability of the electrical transducer to record electrochemical signals with high sensitivity, while also having antimicrobial components to prevent inflammatory responses. The present disclosure addresses some of these needs.

### SUMMARY

In general, various aspects described herein relate to compositions and their application to conducting surfaces and transducer surfaces. The coatings protect these surfaces from unwanted interactions that impede or diminish their intended function. Furthermore, the coatings described herein exhibit antimicrobial activity and prevent adverse immune responses from tissue trauma during implantation/insertion, encapsulation responses that alter analytes accessibility and the surface characteristics of the sensors, and a physiological mismatch between sensor and surrounding tissue.

In one aspect provided herein is a composition, e.g., a mixture comprising a proteinaceous material, a conductive element, and an anti-microbial agent. The mixture can be a heterogenous mixture or a homogenous mixture. For example, the mixture is a homogenous mixture.

The various components of the composition can be cross-linked. For example, the anti-microbial agent is covalently linked to the proteinaceous material or the conductive element. Optionally, the anti-microbial agent is covalently linked to the proteinaceous material or the conductive element via a linker, and optionally, the linker comprises genipin, polyethylene glycol (PEG), or glutaraldehyde. Also optionally, the ratio of anti-microbial agent to cross-linker is from about 100:1 to 10:1; the ratio of the anti-microbial agent to conductive element is from about 30:1 to 10:1; and the ratio of the proteinaceous material to the conductive element is from about 10:1 to 1:1.

Optionally, the anti-microbial agent is an anti-bacterial agent, anti-fungal agent or anti-viral agent. Optionally, the anti-microbial agent comprises an amino (NH₂) group. Also, optionally, the anti-microbial agent is an antimicrobial peptide or polymer, or is a metal particle. For example, the anti-microbial agent is titanium oxide, copper, or silver nanoparticles. In some aspects, the composition comprises at least two (e.g., 3, 4, 5, or more) different anti-microbial agents.

Optionally, the proteinaceous material is denatured or non-reversibly denatured. Optionally, the proteinaceous material is a globular protein, non-glycosylated protein, serum albumin protein, bovine serum albumin (BSA) or human serum albumin (HAS). Optionally, the proteinaceous material is cross-linked with the conductive element. Also optionally, the proteinaceous material is cross-linked to itself. In some aspects, the proteinaceous material is cross-linked to the conductive element or to itself by a linker, for example, genipin, polyethylene glycol, or glutaraldehyde.

Optionally, the conducting material comprises conductive particles, conductive rods, conductive fibers, conductive nano-particles, nano-flakes, nanotubes, conductive polymers, semi-conductive particles, semi-conductive rods, semi-conductive fibers, semi-conductive nano-particles or semi-conductive polymers. Optionally, the conducting material is a metal, a metalloid, a conducting polymer, a conducting carbon based material, or any combination thereof. Optionally, the conducting material comprises an allotrope of carbon atoms arranged in a hexagonal lattice. In some aspects, the conducting material is graphite, functionalised graphene oxide, reduced graphene oxide, or carbon nano-tubes (CNTs), and optionally the conducting material is carboxylated or animated. Optionally, the conductive material comprises gold.

Optionally, the mixture further comprises a target binding molecule capable of binding with a target molecule. Optionally, the target binding molecule is covalently linked to the proteinaceous material, or the conductive or semi-conductive material. In some aspects, the target binding molecule is a receptor, a ligand for a receptor, an antibody, antigen binding fragment of an antibody, an antigen, an enzyme, or a nucleic acid.

Optionally, the composition further comprises a polymer, for example, polyethylene glycol (PEG), alginate, polytetrafluoroethylene (PTFE), polyacetylene (PA), polyaniline (PANI), polypyrrole (PPy), polythiophene (PTH), poly(para-phenylene) (PPP), poly(phenylenevinylene) (PPV), or polyfuran (PF). Optionally, the composition further comprises a drug elutin component, for example, anti-inflammatory drugs sirolimus, everolimus, biolimus (A9), zotarolimus (ABT-578), tacrolimus, and pimecrolimus, genistein, steroids (dexamethasone, prednisolone, methylprednisolone and hydrocortisone), fluocinolone acetonide, hormones etc. Optionally, the mixture is a homogenous mixture and is antifouling and has antimicrobial activity.

In one aspect, provided herein is a surface comprising a composition provided herein coated thereon. with antifouling properties, wherein the surface comprises an antifouling coating layer comprising a composition provided herein. Optionally, the antifouling coating layer is directly or indirectly connected with an electrode.

In another aspect, the is the disclosure provides an electrode comprising (i) a conductive substrate (e.g., an electrically conductive substrate); and (ii) an antifouling coating layer on at least a portion of a surface of the conductive substrate, and wherein the antifouling coating layer comprises a composition provided herein. Optionally, the conductive substrate comprises gold, silver, copper, platinum, aluminum, stainless steel, tungsten, indium tin oxide, titanium, lead, nickel, palladium, zirconium, niobium, tantalum, chromium, molybdenum, manganese, rhenium, ruthenium, rhodium, iridium, silicon, polyimide, parylene, benzocyclobutene, carbon, graphite, or any combination thereof. Also, optionally, the conductive substrate comprises a flexible substrate comprising polyethylene terephthalate, polyethylene naphathalate, polyimides, polymeric hydrocarbons, celluloses, plastics, polycarbonates, polystyrenes, or any combination thereof. Optionally, the antifouling coating layer is adapted for contact with an analyte or a sample comprising an analyte, and the antifouling coating layer has a thickness of from about 0.1 nm to about 10 μm. Also optionally, the electrode is a planar or 3-dimensional electrode.

In yet another aspect, provided herein is an organic field effect transistor comprising: (a) a source electrode; (b) a drain electrode; (c) an organic conducting or semiconducting layer connecting the source electrode to the drain electrode; and (d) an antifouling coating layer on at least a portion of the surface of the organic conducting or semiconducting layer, and wherein the antifouling coating layer comprises a composition provided herein.

Optionally, the organic conducting or semiconducting layer comprises one or more organic compounds having conducting and/or semiconducting properties, and the organic conducting or semiconducting layer has a thickness of from about 0.1 nm to about 10 μm. For example, the organic compounds having conducting and/or semiconducting properties are selected from the group consisting of polyacetylenes, porphyrins, phthalocyanins, fullerenes, polyparaphenylenes, polyphenylenevinylenes, polyfluorenes, polythiophenes, polypyrroles, polypyridines, polycarbazoles, polypyridinevinylenes, polyarylvinylenes, poly (p-phenylmethylvinylenes), derivatives and co-polymers thereof, and any combination thereof. Optionally, the organic field effect transistor further comprises gate electrode in contact with the antifouling coating layer or the organic conducting or semiconducting layer, and the antifouling coating layer is configured to be connected to an ohmic conductor for applying a gate voltage to said organic conducting or semiconducting layer (e.g., via antifouling layer). Also, optionally, the antifouling coating layer has a thickness of from about 0.1 nm to about 10 μm.

In a further aspect, provided herein is a method for detecting a target analyte in a sample, the method comprising: contacting a sample suspected of comprising a target analyte with an electrode or organic field effect transistor and detecting binding of the target analyte with the target binding ligand. Optionally, the detecting the binding of the target molecule with the target binding ligand comprises applying a voltage to the electrode, measuring a current generated from an electrode, applying a voltage to the drain electrode or ohmic conductor, grounding the source electrode, or detecting a drain current. Optionally, the second target binding molecule comprises a detectable label, for example, an enzyme, a fluorophore, a chemiluminescent label, colloidal gold, colored glass or plastic (e.g., polystyrene, polypropylene, and latex) beads, a radiolabel, a quantum dot, or any combination thereof.

Optionally, the target analyte is a peptide, a polypeptide, a peptidomimetic, a nucleic acid, an oligosaccharide, a polysaccharide, an amino acid, nucleoside, a nucleotide, a carbohydrate, a lipid, a peptidoglycan, a cell, microbial matter, an antigen, a lipid, a steroid, a hormone, a lipopolysaccharide, an endotoxin, a therapeutic agent, a lipid-binding molecule, a cofactor, a small molecule, a toxin, a biological threat agent (e.g., spore, viral, cellular and protein toxin), or any combination thereof.

Optionally, the sample is a biological sample (e.g., blood, saliva, amniotic fluid, sputum, urine, semen, cerebrospinal fluid, bronchial aspirate, perspiration, mucus, liquefied stool sample, synovial fluid, lymphatic fluid, tears, tracheal aspirate, lactation product, and any combination thereof); or a food, an ingredient for preparing a food, poultry, meat, fish, beverage, or dairy product; or a non-biological sample (e.g., water, salt water, pond water, river water, reservoir water, brine, drinking water, industrial water, brown water, waste water, sewerage, soil, and mixtures thereof.

In another aspect, provided herein is a sensor comprising an electrode or organic field effect transistor with an antifouling coating provided herein. Optionally, the sensor comprises a fluid-contact surface and the electrode or the organic field effect transistor is immobilized on at least a portion of the fluid-contact surface, and the fluid-contact surface further comprises a positive control electrode and/or a negative control electrode immobilized thereon. Also optionally, the sensor comprises one or more microfluidic flow cells.

In yet another aspect, provided herein is a use of an electrode or organic field effect transistor with an antifouling coating provided herein for detecting a target analyte in a sample.

In another aspect, provided herein is a kit comprising a composition, surface, electrode, organic field effect transistor or sensor provided herein.

### BRIEF DESCRIPTION OF THE DRAWINGS

This patent or application file contains at least one drawing executed in color. Copies of this patent or patent application publication with color drawings will be provided by the Office upon request and payment of the necessary fee.

FIGS. 1A-1B show Schematic of RepelSense nanocomposite synthesis (FIG. 1A) showing ultrasonication of purified BSA and rGOx, BSA heat-denaturation, centrifugation and supernatant collection, addition of antibiotic and cross-linker to supernatant, drop-casting onto a plasma-treated gold electrode, and overnight incubation in a humidity chamber at room temperature (RT). (FIG. 1B) In action, RepelSense nanocomposite prevents attachment of bacteria (green and red rods), proteins (purple) and immune cells (pink cell) and actively kills bacteria, while allowing for specific electrochemical sensing as measured by cyclic voltammetry (CV).

FIGS. 2A-2B show Evaluation of antimicrobial efficacy of RepelSense coatings by FIG. 2A) a micro broth assay using ~10⁶ CFU mL−1 P. aeruginosa grown in LB medium over 15 h at 37° C. A saturating dose of soluble gentamicin (100 μg mL−1) was used as a positive control (Soluble G), and a blank tissue culture plate was used as a negative control (Blank) (n=3). FIG. 2B) Inoculation of coated chips with ~10² CFU mL−1 P. aeruginosa incubated over 24 h at 70 rpm at 37° C., repeated for 3 consecutive days, with RepelSense+covalently bound gentamicin (RepelSense G) and RepelSense+meropenum which has no primary amine groups (RepelSense M).

FIGS. 3A-3F show Monocytes seeded on eRapid and RepelSense, incubated over 24 h, and (FIG. 3A) analyzed by flow cytometry, and (FIG. 3B) an alamarBlue cell viability assay (n=3); the background signal was removed before plotting. We then normalized the alamarBlue signal with monocyte viability (FIG. 3C). (FIG. 3D) Expression of the co-stimulatory molecule CD14 on live cells, (FIG. 3E) HLA-DR on CD14+ cells, and (FIG. 3F) CD83 on CD14+ cells, were then analyzed by flow cytometry (n=2). The negative control was an empty plasma-treated tissue culture plate well (−), and the positive control was polyurethane (PU) film containing 0.1% zinc diethyldithiocarbamate (ZDEC) (+). *: p<0.05, **: p<0.01, ***p<0.001, ****p<0.0001.

FIGS. 4A-4F show Immunofluorescent staining of fibroblasts seeded on (FIG. 4A) RepelSense, (FIG. 4B) RepelSense G, (FIG. 4C) Negative control, (FIG. 4D) eRapid, and (FIG. 4E) the cytotoxic control (polyeurethane (PU) film containing 0.1% zinc diethyldithiocarbamate (ZDEC) (n=3). Images are separated into the channels: (I) ATTO 647 for F-actin, (II) Alexa 488 for a-SMA, (III) Hoechst for DNA, and (IV) Combined. The cytotoxic control was autofluorescent in the Hoechst wavelength (FIG. 4E). (FIG. 4F) alamarBlue viability assay of human-derived fibroblasts immediately after incubation with RepelSense coatings (RepelSense, RepelSense G, RepelSense Cx) and eRapid at 37° C. for 24 h and 48 h (n=3). The negative control was a plasma-treated tissue culture plate well (−), and the positive control was polyurethane (PU) film containing 0.1% zinc diethyldithiocarbamate (ZDEC) (+). The background signal was removed before plotting and the data was normalized. ****: p<0.0001.

FIGS. 5A-5E show Fibroblast cytokine expression of IL-1a, IL-8, IL-6, VEGF, and MCP-1 after 24 h incubation. The negative control was a plasma-treated tissue culture plate well (−), and the positive control was polyurethane (PU) film containing 0.1% zinc diethyldithiocarbamate (ZDEC) (+). The data was normalized by cell count. Expression was upregulated after exposure to eRapid for IL-1a, and was not significant for RepelSense coatings except when compared to RepelSense Cx for IL-6, VEGF, and MCP-1. ****: p<0.0001, *p<0.001, **: p<0.01.

FIGS. 6A-6D show (FIG. 6A) RepelSense coating current densities measured by cyclic voltammetry (CV). (FIG. 6B) Current density remained high for all RepelSense coatings even after 21 d incubation with 1% BSA; no signal was detected on the gold uncoated electrode (n=2). (FIG. 6C) The immunosensor developed with RepelSense and functionalized with capture antibodies (cAb) to detect MIP-1b showed a response in the range of 0.10 to 100 ng mL−1 in RPMI medium (n=3). and (FIG. 6D) with cAbs to detect IL-6 showed a limit of detection of 0.018 ng mL−1 in human plasma (n=3), indicating its high sensitivity even in complex biological samples.

FIGS. 7A-7D show Antimicrobial efficacy of RepelSense coatings with varied antibiotic (G) and crosslinker (GNP) concentrations measured by a micro broth assay using 10⁶ CFU mL−1 P. aeruginosa grown in LB medium over 15 h at 37° C. (n=3). A saturating dose of soluble Gentamicin (100 μg mL−1) was used as a positive control (Soluble G), and a blank tissue culture plate was used as a negative control (Blank).

FIGS. 8A-8F show AlamarBlue assay run immediately on human-derived fibroblasts after 24 h and 48 h incubation with RepelSense coatings (RepelSense, RepelSense G, RepelSense Cx) and eRapid at 37° C.; background signal was removed before plotting. The negative control was a plasma-treated tissue culture plate well (−), and the positive control was polyether urethane (PU) film containing 0.1% zinc diethyldithiocarbamate (ZDEC) (+).

FIG. 9 shows RepelSense coating current densities measured by cyclic voltammetry (CV). The immunosensor with RepelSense was functionalized with capture antibodies (cAb) to detect IL-6 in RPMI medium.

FIGS. 10A-10E show the electrochemical performance and antifouling ability of the antibiotic-crosslinked nanocomposites when tested at concentrations of 1 mg/ml through Cyclic Voltammetry (CV) in 5 mM ferri-ferrocyanide. (FIG. 10A) The current densities of the oxidation peak for the gentamicin-, ampicillin-, and vancomycin nanocomposites (NC-Gent, NC-Amp, NC-Vanc) and after coating after 1 hr and 1 day incubation with 1% BSA, (FIG. 10B) The peak-to-peak separation of each NC after 1 hr and 1 day incubation with 1% BSA; (FIG. 10C) CV of NC-Gent); (FIG. 10D) CV of NC-Amp; (FIG. 10E) CV of NC-Vanc. The coatings are compared to an uncoated gold electrode (Gold).

FIGS. 11A-11D show the antimicrobial efficacy tested through a microdilution assay. (FIG. 11A) Optical density (OD) of Gentamicin-crosslinked nanocomposite (NC-gent), Ag NP nanocomposite (NC-Ag), ZnO NP nanocomposite (NC-ZnO), incubated with Pseudomonas aeruginosa over 15 hours at 37° C. For a positive control 200 μl gentamicin and uncoated wells (blank) were used. (FIG. 11B) Absorbance at 450 nm of various anti-microbial nanocomposites, incubated with Pseudomonas aeruginosa over 15 hours at 37° C. For a positive control 200 μl gentamicin and uncoated wells (blank) were used. (FIG. 11C) Average OD measurements of NC-Gent of concentrations of 1 mg/ml, 5 mg/ml and 10 mg/ml using either the rapid or overnight protocol at 4.5 h; (FIG. 11D) Average OD measurements of gentamicin-, vancomycin-, or ampicillin-crosslinked nanocomposites (NC-Gent, NC-Vanc, NC-Amp) and ZnO Nps in BSA at 4.5 h. The respective antibiotics were added at 200 μl/ml as positive control and BSA and uncoated wells (Blank) were used as negative controls.

FIGS. 12A-12B show Monocyte viability and metabolic activity was determined of the GNP and GTA crosslinked coatings to test the biocompatibility of the crosslinkers. (FIG. 12A) flow cytometry after FACS staining of samples showing the viability of monocytes of GTA and GNP crosslinked composites with either bovine serum albumin (BSA) or human serum albumin (HAS); (FIG. 12B) alamarBlue assay assessing the metabolic activity of the remaining cells. All coatings are compared to a cytotoxic negative and positive control.

FIG. 13 shows an immunoassay against MIP-1b demonstrates the viability of the antibiotic-crosslinked nanocomposites (NC-Vanc, NC-Gent, NC-Amp) to perform equal to the current gold standard of BSA/GOx/GA (NC-BSA/rGOx) coatings for a sensor application.

FIGS. 14A and 14B show the antimicrobial efficiency of antibiotics with amine groups. 40 μl of P. aeruginosa in LB of approximate concentrations of 10² CFU/ml, incubated in humidity chamber 24 hr with shaker, 70 rpm, at 37 C were grown in the presence of Genipin with amine groups, Meropenum without amino groups, or without antibiotics.

FIG. 15 is the experimental step for an elution study comparing antibiotics with amine groups and without. 40 μl LB broth was added to tape well of chip, and it was incubated in humidity chamber 24 hr, removed and place on a pre-swabbed bacterial lawn. Bacterial growth was observed after 1, 2, and 3 days.

FIGS. 16A-16C show results of an elution study comparing antibiotics with amine groups and without. Bacterial growth on day 1 (FIG. 16A), day 2 (FIG. 16B) and day 3 (FIG. 16C) surrounding chips coated with Genipin with amine groups, Meropenem without amine groups, and no antibiotic.

FIGS. 17A-17K show microscopy and immunofluorescent staining for cytotoxicity against fibroblasts of various versions of an antimicrobial biosensor. Cytotoxicity was assessed after 24 and 48 hrs. All wells were washed carefully and stained for F-actin, ATTO 647 red, a-SMA, ALEXA 488 gree, and DNA/nucleus, Hoechsht-Dapi blue). (FIG. 17A) A regular tissue culture well, which served as a negative control. (FIG. 17B) Culture well containing a cytotoxic polyether urethane (PU) film containing 0.1% zinc diethyldithiocarbamate (ZDEC), which served as a positive control. (FIG. 17C) Culture well containing biosensor coated with human serum albumin crosslinked with glutaraldehyde. (FIG. 17D) Culture well containing biosensor coated with human serum albumin crosslinked with genipin. (FIG. 17E) Culture well containing biosensor coated with bovine serum albumin crosslinked with glutaraldehyde. (FIG. 17F) Culture well containing biosensor coated with bovine serum albumin crosslinked with genipin. (FIG. 17G) Culture well containing biosensor coated with bovine serum albumin crosslinked with genipin and containing gentamicin. (FIG. 17H) Culture well containing biosensor coated with bovine serum albumin crosslinked with genipin and containing ampicillin. (FIG. 17I) Culture well containing biosensor coated with bovine serum albumin crosslinked with genipin and containing cefepime cefepime. (FIG. 17J) Culture well containing biosensor coated with bovine serum albumin crosslinked with genipin and containing ceftriaxone. (FIG. 17K) Culture well containing biosensor coated with bovine serum albumin crosslinked with genipin and containing vancomycin.

FIGS. 18A-18B show statistical analysis of immunofluorescent images from FIGS. 17A-17K of smooth muscle alpha-actin. (FIG. 18A) Mean area of smooth muscle alpha actin (aSMA) per culture well containing controls or biosensors with various coatings. (FIG. 18B) Oneway means comparisons with control using Dunnett's method.

FIGS. 19A-19B show statistical analysis of immunofluorescent images from FIG. 8 of filamentous actin. (FIG. 19A) Mean area of filamentous actin (F-Actin) per culture well containing controls or biosensors with various coatings. (FIG. 19B) Oneway means comparisons with control using Dunnett's method.

FIGS. 20A-20C show statistical analysis of immunofluorescent images from FIG. 8 of DNA/Nuclei. (FIG. 20A) Mean area and sphericity of DNA/Nuclei per culture well containing controls or biosensors with various coatings. (FIG. 20B) Oneway means comparisons of mean area of DNA with control using Dunnett's method. (FIG. 20C) Oneway means comparisons of sphericity of nuclei.

FIGS. 21A-21C show the electrochemical performance of the antibiotic-crosslinked nanocomposites. (FIG. 21A) The peak-to-peak separation of each NC after 1 hr and 1 day incubation with 1% BSA; (FIG. 21B) Cyclic Voltammetry (CV) in 5 mM ferri-ferrocyanide of NC-GNP-Amp, NC-GNP-Gent, and NC-GNP-Vanc. The coatings are compared to an uncoated gold electrode (Gold). (FIG. 21C) Effect on electrochemical performance of the order of adding antibiotic on the NC before or after crosslinking.

### DETAILED DESCRIPTION

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose The methods, compositions, and structures provided herein are based in part on the preparation of composition, e.g., a mixture comprising a proteinaceous material, a conductive element, and an anti-microbial agent.

### Anti-microbial Agents

Embodiments of the various aspects described include an antimicrobial agent. The term “antimicrobial agent” as used herein refers to any entity with antimicrobial activity, i.e. the ability to inhibit or reduce the growth and/or kill a microbe, e.g., by at least about 30%, at least about 40%, at least about 50%, at least about 75%, at least about 90% or more, as compared to in the absence of an antimicrobial agent.

In some embodiments, the anti-microbial agent is an anti-bacterial agent, anti-fungal agent, or an anti-viral agent.

In some embodiments, an antimicrobial agent included in the composition can be an antibiotic. As used herein, the term “antibiotic” is art recognized and includes antimicrobial agents naturally produced by microorganisms such as bacteria (including Bacillus species), actinomycetes (including Streptomyces) or fungi that inhibit growth of or destroy other microbes, or genetically-engineered thereof and isolated from such natural source. Substances of similar structure and mode of action can be synthesized chemically, or natural compounds can be modified to produce semi-synthetic antibiotics. Exemplary classes of antibiotics include, but are not limited to, (1) β-lactams, including the penicillins, cephalosporins monobactams, methicillin, and carbapenems; (2) aminoglycosides, e.g., gentamicin, kanamycin, neomycin, tobramycin, netilmycin, paromomycin, and amikacin; (3) tetracyclines, e.g., doxycycline, minocycline, oxytetracycline, tetracycline, and demeclocycline; (4) sulfonamides (e.g., mafenide, sulfacetamide, sulfadiazine and sulfasalazine) and trimethoprim; (5) quinolones, e.g., ciprofloxacin, norfloxacin, and ofloxacin; (6) glycopeptides (e.g., vancomycin, telavancin, teicoplanin); (7) macrolides, which include for example, erythromycin, azithromycin, and clarithromycin; (8) carbapenems (e.g., ertapenem, doripenem, meropenem, and imipenem); (9) cephalosporins (e.g., cefadroxil, ceftriaxone, cefepime, and ceftobiprole); (10) lincosamides (e.g., clindamycin, and lincomycin); (11) monobactams (e.g., aztreonam); (12) nitrofurans (e.g., furazolidone, and nitrofurantoin); (13) Penicillins (e.g., amoxicillin, and Penicillin G); (14) polypeptides (e.g., bacitracin, colistin, and polymyxin B); and (15) other antibiotics, e.g., ansamycins, polymycins, carbacephem, chloramphenicol, lipopeptide, and drugs against mycobacteria (e.g., the ones causing diseases in mammals, including tuberculosis (Mycobacterium tuberculosis) and leprosy (Mycobacterium leprae), and any combinations thereof. Additional exemplary antimicrobial agent can include, but are not limited to, antibacterial agents, antifungal agents, antiprotozoal agents, antiviral agents, and any mixtures thereof.

In some embodiments of any one of the aspects described herein the anti-microbial agent is an anti-bacterial agent. Exemplary antibacterial agents include, but are not limited to, Acrosoxacin, Amifioxacin, Amoxycillin, Ampicillin, Aspoxicillin, Azidocillin, Azithromycin, Aztreonam, Balofloxacin, Benzylpenicillin, Biapenem, Brodimoprim, Cefaclor, Cefadroxil, Cefatrizine, Cefcapene, Cefdinir, Cefetamet, Cefmetazole, Cefprozil, Cefroxadine, Ceftibuten, Cefuroxime, Cephalexin, Cephalonium, Cephaloridine, Cephamandole, Cephazolin, Cephradine, Chlorquinaldol, Chlortetracycline, Ciclacillin, Cinoxacin, Ciprofloxacin, Clarithromycin, Clavulanic Acid, Clindamycin, Clofazimine, Cloxacillin, Danofloxacin, Dapsone, Demeclocycline, Dicloxacillin, Difloxacin, Doxycycline, Enoxacin, Enrofloxacin, Erythromycin, Fleroxacin, Flomoxef, Flucloxacillin, Flumequine, Fosfomycin, Isoniazid, Levofloxacin, Mandelic Acid, Mecillinam, Metronidazole, Minocycline, Mupirocin, Nadifloxacin, Nalidixic Acid, Nifuirtoinol, Nitrofurantoin, Nitroxoline, Norfloxacin, Ofloxacin, Oxytetracycline, Panipenem, Pefloxacin, Phenoxymethylpenicillin, Pipemidic Acid, Piromidic Acid, Pivampicillin, Pivmecillinam, Prulifloxacin, Rufloxacin, Sparfloxacin, Sulbactam, Sulfabenzamide, Sulfacytine, Sulfametopyrazine, Sulphacetamide, Sulphadiazine, Sulphadimidine, Sulphamethizole, Sulphamethoxazole, Sulphanilamide, Sulphasomidine, Sulphathiazole, Temafioxacin, Tetracycline, Tetroxoprim, Tinidazole, Tosufloxacin, Trimethoprim, and pharmaceutically acceptable salts or esters thereof.

In some embodiments of any one of the aspects described herein, the anti-bacterial agent is selected from the group consisting of macrolides or ketolides such as erythromycin, azithromycin, clarithromycin, and telithromycin; beta-lactams including penicillin, cephalosporin, and carbapenems such as carbapenem, imipenem, and meropenem; monolactams such as penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, cefiriaxone, cefoperazone, ceftazidime, cefixime, cefpodoxime, ceftibuten, cefdinir, cefpirome, cefepime, cefadroxil, ceftriaxone, ceftobiprole and astreonam; quinolones such as nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, ganefloxacin, gemifloxacin and pazufloxacin; antibacterial sulfonamides and antibacterial sulphanilamides, including para-aminobenzoic acid, sulfadiazine, sulfisoxazole, sulfamethoxazole and sulfathalidine; aminoglycosides such as streptomycin, neomvcin, kanamycin, paromycin, gentamicin, tobramycin, amikacin, netilmicin, spectinomycin, sisomicin, dibekalin and isepamicin; tetracyclines such as tetracycline, chlortetracycline, demeclocycline, minocycline, oxytetracycline, methacycline, doxycycline; rifamycins such as rifampicin (also called rifampin), rifapentine, rifabutin, bezoxazinorifamycin and rifaximin; lincosamides such as lincomycin and clindamycin; glycopeptides such as vancomycin and teicoplanin; streptogramins such as quinupristin and daflopristin; oxazolidinones such as linezolid; polymyxin, colistin and colymycin; trimethoprim, bacitracin, and phosphonomycin. In some preferred embodiments, the antibacterial agent is gentamicin, ampicillin, vancomycin, ceftriaxone or cefepime.

In some embodiments of any one of the aspects described herein the anti-microbial agent is an anti-fungal agent. Exemplary antifungal agents include, but are not limited to, Bifonazole, Butoconazole, Chlordantoin, Chlorphenesin, Ciclopirox Olamine, Clotrimazole, Eberconazole, Econazole, Fluconazole, Flutrimazole, Isoconazole, Itraconazole, Ketoconazole, Miconazole, Nifuroxime, Tioconazole, Terconazole, Undecenoic Acid, and pharmaceutically acceptable salts or esters thereof. In some embodiments of any one of the aspects described herein, the anti-fungal agent is selected from the group consisting of azoles (e.g., barleyconazole, butoconazole, clortrimazole, econazole, fluconazole, isavuconazole, itraconazole, ketoconazole, miconazole, oxyconazole, posaconazole, ravuconazole, saperconazole, sulconazole, tercocnazole, tioconazole, voriconazole, and ciclopirox), polyenes (e.g., natamycin, lucensomycin, nystatin, amphotericin B, etc.), echinocandins (e.g., Cancidas), pradimicins (e.g., beanomicins, nikkomycins, sordarins, allylamines, etc.), Triclosan, Piroctone, fenpropimorph, terbinafine, cyclopyroxolamine, flucitocin, griseofulvin haloprozin, tolnaftate, naphthypine, hydrochloride, morpholine, butenapin, undecylenic acid, propionic acid, and derivatives and analogs thereof.

In some embodiments of any one of the aspects described herein, the anti-microbial agent is an antiprotozoal agent. Exemplary antiprotozoal agents include, but are not limited to, Acetarsol, Azanidazole, Chloroquine, Metronidazole, Nifuratel, Nimorazole, Omidazole, Propenidazole, Secnidazole, Sinefingin, Tenonitrozole, Temidazole, Tinidazole, and pharmaceutically acceptable salts or esters thereof.

In some embodiments of any one of the aspects described herein, the anti-microbial agent is an antiviral agent. Exemplary antiviral agents include, but are not limited to, Acyclovir, Brivudine, Cidofovir, Curcumin, Desciclovir, 1-Docosanol, Edoxudine, Fameyclovir, Fiacitabine, Ibacitabine, Imiquimod, Lamivudine, Penciclovir, Valacyclovir, Valganciclovir, and pharmaceutically acceptable salts or esters thereof.

An antimicrobial agent can be, for example, but not limited to, a small molecule, a peptide, a peptidomimetics, an antibody or a fragment thereof, a nucleic acid, an enzyme (e.g., an antimicrobial metalloendopeptidase such as lysostaphin), an aptamer, a drug, an antibiotic, a chemical or any entity that can inhibit the growth and/or kill a microbe. In some embodiments of the any one of the aspects described herein, the anti-microbial agent is an antimicrobial peptide or polymer. Examples of antimicrobial peptides include, but are not limited to, mefloquine, venturicidin A, antimycin, myxothiazol, stigmatellin, diuron, iodoacetamide, potassium tellurite hydrate, aDL-vinylglycine, N-ethylmaleimide, L-allyglycine, diaryquinoline, betaine aldehyde chloride, acivcin, psicofuraine, buthionine sulfoximine, diaminopemelic acid, 4-phospho-D-erythronhydroxamic acid, motexafin gadolinium, xycitrin, cathelicidins, defensins, protegrins, mastoparan, poneratoxin, cecropin, moricin, melittin, magainin, dermaseptin, and/or nisin, or modified versions or analogues thereof.

In some embodiments, the anti-microbial agent is a metal particle. Exemplary metal particles can include silver, titanium oxide, or copper present in any form, e.g., a nanoparticle, a colloid, a suspension, powder, and any combinations thereof. In some embodiments of any one of the aspects described herein the anti-microbial agent is titanium oxide, copper, or silver nanoparticles.

It is noted that compositions and mixtures described herein can comprise two or more different antimicrobial agents. Accordingly, in some embodiments of any one of the aspects described herein, the composition or mixture described herein comprises more than one (e.g., at least 2, at least 3, at least 4, at least 5, at least 6, at least 7, at least 8, at least 9, at least 10, or more) different anti-microbial agents. The different anti-microbial agents can be for the same indication, e.g., anti-bacterial, or for different indications, e.g., one anti-microbial agent in the mixture is an anti-bacterial agent and another anti-microbial agent in the mixture is an anti-fungal agent.

The anti-microbial agent can comprise a functional group for cross-linking. Exemplary functional groups amenable to cross-linking include, but are not limited to, amino, hydroxyl, alkoxy, carbonyl, carboxyl, silyl, silyloxy, hydrocarbyl, sulfhydryl, cycloalkyl, aryl, thio, mercapto, imino, halo, cyano, nitro, azido, sulfoxy, phosphoryl, oxy, quinone, catechol, and the like. In some embodiments of any one of the aspects described herein, the anti-microbial agent comprises at least one amino (NH₂) group.

In some embodiments of any one of the aspects described herein, the anti-microbial agent is covalently linked to the proteinaceous material or the conductive element. For example, the anti-microbial agent is linked to the proteinaceous material or the conductive element via a linker or cross-linker. In some embodiments of any one of the aspects described herein, the anti-microbial agent is covalently linked to the proteinaceous material via a cross-linking agent such as genipin, polyethylene glycol (PEG), or glutaraldehyde. In some preferred embodiments, the anti-microbial agent is covalently linked to the proteinaceous material via genipin.

In some embodiments of any one of the aspects described herein, the anti-microbial agent is covalently linked to the conductive element via a cross-linking agent such as genipin, polyethylene glycol (PEG), or glutaraldehyde. In some preferred embodiments, the anti-microbial agent is covalently linked to the the conductive element via genipin The ratio of the anti-microbial agent to the cross-linking agent can be from about 100:1 to about 1:1 (w/w). In some embodiments of any one of the aspects described herein, the ratio of the anti-microbial agent to the cross-linker is from about 100:1 to about 10:1 (w/w). For example, the ratio of the anti-microbial agent to the cross-linker can be from about 90:1 to about 20:1 w/w, about 80:1 to about 30:1 w/w, about 70:1 to about 40:1 w/w, or about 60:1 to about 50:1 w/w. In some embodiments, the w/w ratio of anti-microbial agent to cross-linker is about 100:1, or about 95:1, or about 90:1, or about 85:1, or about 80:1, about 75:1, or about 70:1, or about 65:1, or about 60:1, about 55:1, or about 50:1, or about 45:1, or about 40:1, about 35:1, or about 30:1, or about 25:1, or about 20:1, about 15:1, or about 10:1.

Similarly, the ratio of the anti-microbial agent to the proteinaceous material can be from about 30:1 to about 1:1 (w/w). In some embodiments of any one of the aspects described herein, the ratio of the anti-microbial agent to the proteinaceous material is from about 25:1 to about 2:1 w/w, about 20:1 to about 3:1 w/w, about 15:1 to about 4:1 w/w, or about 10:1 to about 5:1 w/w. In some embodiments, the w/w ratio of anti-microbial agent to proteinaceous material is about 30:1, or about 29.5:1, or about 29:1, or about 28.5:1, or about 28:1, or about 27.5:1, or about 27:1, or about 26.5:1, or about 26:1, or about 25.5:1, or about 25:1, or about 24.5:1, or about 24:1, or about 23.5:1, or about 23:1, or about 22.5:1, or about 22:1, or about 21.5:1, or about 21:1, or about 20.5:1, or about 20:1, or about 19.5:1, or about 19:1, or about 18.5:1, or about 18:1, or about 17.5:1, or about 17:1, or about 16.5:1, or about 16:1, or about 15.5:1, or about 15:1, or about 14.5:1, or about 14:1, or about 13.5:1, or about 13:1, or about 12.5:1, or about 12:1, or about 11.5:1, or about 11:1, or about 10.5:1, or about 10:1, or about 9.5:1, or about 9:1, or about 8.5:1, or about 8:1, or about 7.5:1, or about 7:1, or about 6.5:1, or about 2.5:1, or about 2:1, or about 1.5:1, or about 1:1.

The ratio of the anti-microbial agent to the conductive element can be from about 20:1 to 1:1 (w/w). In some embodiments of any one of the aspects described herein, the ratio of the anti-microbial agent to the conductive element is from about 20:1 to about 5:1. For example, the ratio of the anti-microbial agent to the conductive element can be from about 15:1 to 5:1 w/w, 12.5:1 to 3:1 w/w, or 5:1 to 4:1 w/w. In some embodiments, the w/w ratio of anti-microbial agent to the conductive element is about 20:1, or about 19.5:1, or about 19:1, or about 18.5:1, or about 18:1, or about 17.5:1, or about 17:1, or about 16.5:1, or about 16:1, or about 15.5:1, or about 15:1, or about 14.5:1, or about 14:1, or about 13.5:1, or about 13:1, or about 12.5:1, or about 12:1, or about 11.5:1, or about 11:1, or about 10.5:1, or about 10:1, or about 9.5:1, or about 9:1, or about 8.5:1, or about 8:1, or about 7.5:1, or about 7:1, or about 6.5:1, or about 6:1, or about 5.5:1, or about 5:1.

The amount of the antimicrobial agent in the composition can range from about 1 μg/μL to about 100 μg/μL. For example, the amount of the antimicrobial agent in the composition can be about 1 μg/μL, about 5 μg/μL, about 10 μg/μL, about 15 μg/μL, about 20 μg/μL, about 25 μg/μL, about 30 μg/μL, about 35 μg/μL, about 40 μg/μL, about 45 μg/μL, about 50 μg/μL, about 55 μg/μL, about 60 μg/μL, about 65 μg/μL, about 70 μg/μL, about 75 μg/μL, about 80 μg/μL, about 85 μg/μL, about 90 μg/μL, about 95 μg/μL, or about 100 μg/μL. In some embodiments, the amount of the antimicrobial agent in the composition can range from about 1 μg/μL to about 50 μg/μL. For example, the amount of the antimicrobial agent in the composition can range from about 1 μg/μL to about 50 μg/μL, from about 5 μg/μL to about 25 μg/μL, or from about 10 μg/μL to about 20 μg/μL.

### Proteinaceous Material

As used herein, the term “proteinaceous” refers to proteins, peptides and the like. Generally, proteinaceous material includes proteins and peptides, functionalized proteins, copolymers including proteins, natural and synthetic variants of these, and mixtures of these.

In some embodiments, the proteinaceous material is a globular protein. Exemplary globular proteins include, but are not limited to, albumin, Immunoglobulin G (IgG), Immunoglobulin E (IgE), Protein A, avidins, and carbonic anhydrase.

It is noted that the proteinaceous material can be a glycosylated protein or a non-glycosylated protein.

In some embodiments of any one of the aspects described herein, the proteinaceous material is a serum albumin. For example, the proteinaceous material can be Bovine Serum Albumin (BSA) or human serum albumin (HAS). In some embodiments of any one of the aspects described herein, the proteinaceous material is BSA.

In some embodiments, the proteinaceous material is denatured. As used herein, “denaturing” is the process of modifying the quaternary, tertiary and secondary molecular structure of a protein from its natural, original or native state. For example, such as by breaking weak bonds (e.g., hydrogen bonds), which are responsible for the highly ordered structure of the protein in its natural state. The process can be accomplished by, for example: physical means, such as by heating, sonication or shearing; by chemical means such as acid, alkali, inorganic salts and organic solvents (e.g., alcohols, acetone or chloroform); and by radiation. A denatured protein, such as an enzyme, losses its original biological activity. In some instances, the denaturing process is reversible, such that the protein molecular structure is regained by the re-forming of the original bonding interactions at least to the degree that the original biological function of the protein is restored. In other instances, the denaturing process is irreversible or non-reversible, such that the original and biological function of the protein is not restored. Cross-linking, for example after denaturing, can reduce or eliminate the reversibility of the denaturing process.

The degree of denaturing can be expressed as a percent of protein molecules that have been denatured, such as a mole percent. Some methods of denaturing can be more efficient than others. For example, under some conditions, sonication applied to a protein, e.g., serum albumin such as BSA or HSA can denature about 30-40% of the protein and the denaturing is reversible. When BSA is denatured it undergoes two structural stages. The first stage is reversible whilst the second stage is irreversible (e.g., non-reversible) but does not necessarily result in a complete destruction of the ordered structure. For example, heating up to 65° C. can be regarded as the first stage, with subsequent heating above that as the second stage. At higher temperatures, further transformations are seen. In some embodiments, the proteinaceous material e.g., serum album such as BSA or HAS is denatured by heating above about 65° C. (e.g., above about 70° C., above about 80° C., above about 90° C., above about 100° C., above about 110° C., above about 120° C.), below about 200° C. (below about 190° C., 180° C., 170° C., 160° C., 150° C.), and for at least about 1 minute (e.g., at least about 2, 3, 4, 5, 10 or 20 minutes) but less than about 24 hours (e.g., less than about 12, 10, 8, 6, 4, 2 1 hour). According to some implementations, any ranges herein described, for example heating at about 90° C. but below about 150° C. and for at least about 1 minute but less than one hour. As previously noted, the heating can include be included as a separate step to heating of the substrate and include different temperature ranges and heating times.

In some embodiments of any one of the aspects, denaturing of the proteinaceous material can occur before deposition of the mixture on a substrate surface. In some embodiments of any one of the aspects, denaturing can occur only upon deposition on the substrate surface, for example when only a heating step to heat the substrate is included. In some implementations, denaturing occurs before and after deposition, for example, where heating occurs before and after deposition of the mixture on the substrate surface.

In some embodiments the proteinaceous material used in the compositions and structures described herein are at least about 20% to about 100% (e.g., 20%, 30%, 40%, 50%, 60%, 70%, 80%, 90%, or 100%) denatured. In some embodiments, less than 50% of the denatured protein reverts back to its natural state (e.g., less than 40%, less than 30%, less than 20%, less than 10%, less than 1%). Therefore, the reversibility of the denaturing can be described as being 50% reversible, 40% reversible (60% irreversible), 30 % reversible (70% irreversible), 20% reversible (80% irreversible), 10% reversible (90% irreversible) or even 0% reversible (100% irreversible).

In some embodiments, the proteinaceous material is irreversibly or non-reversibly denatured.

The proteinaceous material can be cross-linked with other components present in the composition (i.e., the mixture). For example, the proteinaceous material can be cross-linked with the antimicrobial agent, the conductive element, or itself. Accordingly, in some embodiments, the proteinaceous material is cross-linked with the conductive element. For example, the proteinaceous material is cross-linked to the conductive element by a cross-linking agent. In some embodiments of any one of the aspects described herein, the proteinaceous material is cross-linked to the conductive element by a cross-linking agent selected from genipin, polyethylene glycol, and glutaraldehyde. For example, the proteinaceous material is cross-linked to the conductive element by genipin.

In some embodiments, the proteinaceous material is cross-linked to itself. For example, the proteinaceous material is cross-linked to the conductive element by a cross-linking agent. In some embodiments of any one of the aspects described herein, the proteinaceous material is cross-linked to itself via a cross-linking agent selected from genipin, polyethylene glycol, and glutaraldehyde. For example, the proteinaceous material is cross-linked to itself via genipin.

The ratio of the proteinaceous material to the cross-linking agent can be from about 100:1 to about 1:1 (w/w). In some embodiments of any one of the aspects described herein, the ratio of the proteinaceous material to the cross-linker is from about 100:1 to about 10:1 (w/w). For example, the ratio of the proteinaceous material to the cross-linker can be from about 90:1 to about 20:1, about 80:1 to about 30:1, about 70:1 to about 40:1, or about 60:1 to about 50:1 (w/w). In some embodiments, the w/w ratio of proteinaceous material to cross-linker is about 100:1, or about 95:1, or about 90:1, or about 85:1, or about 80:1, about 75:1, or about 70:1, or about 65:1, or about 60:1, about 55:1, or about 50:1, or about 45:1, or about 40:1, about 35:1, or about 30:1, or about 25:1, or about 20:1, or about 15:1, or about 10:1, or about 5:1 or about 1:1.

The ratio of proteinaceous material to the conductive element can be from about 10:1 to 1:1 (w/w). For example, the w/w ratio of ratio of the proteinaceous material to the conductive element is about 10:1, or about 9.5:1, or about 9:1, or about 8.5:1, or about 8:1, or about 7.5:1, or about 8:1, or about 6.5:1, or about 6:1 or about 5.5:1, or about 5:1, or about 4.5:1, or about 4:1, or about 3.5:1 or about 3:1, or about 2.5:1, or about 2:1, or about 1.5:1 or about 1:1 (w/w).

The amount of the proteinaceous material in the composition can range from about 1 mg/ml to about 20 mg/ml. For example, the amount of the proteinaceous material in the composition can be about 1 mg/ml, about 1.5 mg/ml, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, about 10 mg/ml, about 10.5 mg/ml, about 11 mg/ml, about 11.5 mg/ml, about 12 mg/ml, about 12.5 mg/ml, about 13 mg/ml, about 13.5 mg/ml, about 14 mg/ml, about 14.5 mg/ml, about 15 mg/ml, about 15.5 mg/ml, about 16 mg/ml, about 16.5 mg/ml, about 17 mg/ml, about 17.5 mg/ml, about 18 mg/ml, about 18.5 mg/ml, about 19 mg/ml, about 19.5 mg/ml, or about 20 mg/ml. In some embodiments of any one of the aspects described herein, the amount of the proteinaceous material in the composition is from about 2 mg/ml to about 18 mg/ml, about 3 mg/ml to about 17 mg/ml, about 4 mg/ml to about 16 mg/ml. For example, the amount of the proteinaceous material in the composition is from about 5 mg/ml to about 15 mg/ml. In some embodiments, amount of the proteinaceous material in the composition is from about 5 mg/ml to about 10 mg/ml.

### Conductive Element

Embodiments of the various aspects described herein include a conductive element. As used herein a conductive element is a substance or substrate that has the capability to conduct electricity. The conductive element can comprise conducting and/or semiconducting materials. Further, the conductive element can be in any desired shape or form. For example, the conductive element can be in form of particles (e.g., nanoparticles), rods, flakes (e.g., nanoflakes), tubes (e.g., nanotubes), fibers, sheets, films, and the like. For example, the conductive element can be included in the form of a particle, a nano-particle, a micro-particle, a fiber, a nano-fiber, a micro-fiber, a flake, a nanoflake, a microflake, a tube, a nanotube, a microtube, a crystal, a nanocrystal, a microcrystal, a wire, a nano-wire, a micro-wire, a rod, a nano-rod, a micro-rod, a foil, a sheet, a web, or any combinations of these forms.

The conductive element can be formed from one or more metals, e.g., copper, gold, silver, platinum, palladium, indium, iridium, rhodium, ruthenium, osmium, nickel, tin, titanium, tantalum, tungsten, chromium, iron, aluminum, zinc, combinations thereof, or alloys of any of the foregoing. In addition, or in the alternative, a nonmetallic conductive material can be used. Exemplary nonmetallic conductive materials include, but are not limited to, graphite or acetylene black, graphene, conductive ceramics such as indium tin oxide (ITO), titanium nitride, tungsten nitride, tantalum nitride, and conductive polymers such as polythiophenes, polyanilines, polypyrroles, and polyetheylenes and their mixtures and derivatives.

In some embodiments of any one of the aspects, the conductive element comprises a metal or a metalloid. For example, the conductive element comprises gold. In some embodiments of any one of the aspects described herein, the conductive element comprises gold particles (e.g., gold nano-particles), gold wires (e.g., gold nanowires), gold rods (e.g., gold nano-rods), or any combinations thereof.

In some embodiments of any one of the aspects described herein, the conductive element comprises a conducting carbon based material. For example, the conductive element comprises an allotrope of carbon atoms arranged in a hexagonal lattice. The allotropes of carbon can include some functionalization, such as oxygen, carboxylates, epoxides, amines, amides and combinations of these, as described below. In some implementations, the functionalization includes poly amine functionalization such as pentaamine functionalization. In some embodiments, the conductive element comprises graphite, graphene, graphene oxide, functionalized graphene oxide, reduced graphene oxide (rGO), functionalized reduced graphene oxide, or carbon nano-tubes (CNTs).

As used herein “carbon nanotubes” and “graphene” are allotropes of carbon with sp² carbon atoms arranged in a hexagonal, honeycomb lattice. Single layer graphene is a two-dimensional material, and is a single layer of graphite. As used herein, more than one layer of graphene can be referred to as graphene, for example between 1 and 200 layers (e.g., about 1 to 100 layers, about 1 to 50 layers, about 1 to 10 layers). Carbon nanotubes are hollow, cylindrical structures, formed as a sheet of graphene rolled into a cylinder.

As used herein “graphene oxide” is a material that can be formed from the oxidation of graphene or exfoliation of graphite oxide. In a first step for producing graphene oxide, graphite is oxidized. Several methods for oxidation are known, one common method known as the Hummers and Offeman method, in which graphite is treated with a mixture of sulphuric acid, sodium nitrate and potassium permanganate (a very strong oxidizer). Other methods are known to be more efficient, reaching levels of 70% oxidisation, by using increased quantities of potassium permanganate, and adding phosphoric acid combined with the sulphuric acid, instead of adding sodium nitrate. Exfoliation of graphene oxide provides graphite oxide and can be done by several methods. Sonication can be a very time-efficient way of exfoliating graphite oxide, and it is extremely successful at exfoliating graphene (almost to levels of full exfoliation), but it can also heavily damage the graphene flakes, reducing them in surface size from microns to nanometres, and also produces a wide variety of graphene platelet sizes. Mechanically stirring is a much less destructive approach, but can take much longer to accomplish.

Graphite oxide and graphene oxide are very similar, chemically, but structurally, they are very different. Both are compounds having carbon, oxygen and hydrogen in variable ratios. In the most oxidized state the oxygen amount can be as high as about 60 wt %. the amount of hydrogen varies depending on the functionalization, for example, the number of epoxy bridges, hydroxyl groups and carboxyl groups. The main difference between graphite oxide and graphene oxide is the interplanar spacing between the individual atomic layers of the compounds, caused by water intercalation. This increased spacing, caused by the oxidization process, also disrupts the sp² bonding network, meaning that both graphite oxide and graphene oxide are often described as electrical insulators.

Reduced graphene oxide (rGO) is prepared from reduction of graphene oxide by thermal, chemical or electrical treatments. For example, treating the graphene oxide with; hydrazine, hydrogen plasma, heating in water, high temperature heating (e.g., under nitrogen/argon) and electrochemical reduction. Whereas graphene can be a single carbon layer ideally comprising only carbon, reduced graphene oxide is similar but contains some degree of oxygen functionalization. The amount of oxygen depends on the degree of reduction and in some materials can vary between about 50 wt % and about 1 wt. % (e.g., between about 30 wt. % and about 5 wt. %).

Reduced graphene oxide can be functionalized or include functional groups. For example, reduced graphene oxide often includes oxygen in the form of carboxyl groups and hydroxyl groups. In some forms, the carboxyl and hydroxyl groups populate the edges of the rGO sheets, which can be functionalized. Accordingly, in some embodiments, the reduced graphene oxide (rGO) is carboxylated reduced graphene oxide or aminated reduced graphene oxide. As used herein, carbonylated reduced graphene oxide can refer to reduced graphene oxide having carboxyl groups. In some embodiments the amount of oxygen attributable to the carboxyl groups is between about 30 wt. % and about 0.1 wt. % (e.g., between about 10 wt. % and about 1 wt. %). Other forms of functionalization are possible. For example, amine functionalized rGO can be formed by a modified Buchere reaction, wherein ammonia an graphene oxide are reacted using a catalyst such as sodium bisulfite, or epoxide groups on graphene oxide can be opened with p-phenylenediamine. In some embodiments, the amount of nitrogen is between about 30 wt. % and 0.1 wt. % (e.g., between about 10 wt. % and 1 wt. %). In some implementations, a polyamine is used to functionalize rGO. For example, pentaamine functionalized graphene is used in some implementations.

The tube-shaped carbon nanotubes have diameters in the nanometer scale, such as, for example, between about 0.2 and about 20 nm, preferably between about 0.5 and about 10 nm, and more preferably still between about 1 and about 5 nm. These can be single walled carbon nanotubes (SWCNT), multi walled carbon nanotubes (MWCNT) (e.g., a collection of 2 or more nested tubes of continuously increasing diameters, or mixtures of these). The diameters of MWCNT can be larger than the SWCNT, such as between about 1 and about 100 nm (e.g., between about 1 and about 50 nm, between about 10 and 20 nm, between 5 and 15 nm, between about 30 and 50 nm). Depending on how the precursor graphene sheet is rolled up to make a seamless cylinder that is the carbon nanotube, different isomers of carbon nanotube can be made, for example designated as armchair configuration, chiral configuration, and zigzag configuration. In some embodiments, the carbon nanotubes are carboxylated carbon nanotubes or aminated carbon nanotubes.

The carbon nanotubes and reduced graphene oxide can include intercalated materials, such as ions and molecules. In some embodiments the carbon nanotubes can be functionalized for example by oxidation to form carboxylic acid groups on the surface, providing CNTs. In addition, in some embodiments, the carbon nanotubes and rGO can be further modified through condensation reactions with the carboxylic acid groups present on the CNTs or rGO (e.g., with alcohols and amines), electrostatic interactions with the carboxylic acid groups (e.g., calcium mediated coupling, or quaternary amines, protonated amine-carboxylate interaction, through cationic polymers or surfactants) or hydrogen bonding through the carboxylic acid groups (e.g., with fatty acids, and other hydrogen bonding molecules). The functionalization can be partial (e.g., wherein less than 90%, less than 80%, less than 60%, less than 50%, less than 40%, less than 30%, less than 20%, less than 10%, more than 10%, more than 20%, more than 30%, more than 40%, more than 50%, more than 60%, more than 70%, more than 80%, of the available carboxylic acid groups are functionalized) or complete, such as functionalizing substantially all the carboxylic acids (e.g., more than 90%, more than 95%, more than 99% of available carboxylic acid groups).

In some embodiments of any one of the aspects, the conductive element comprises a conductive polymer. Exemplary conductive polymers include, but are not limited to, polyacrylonitrile (PAN), polyanilines, polypyrroles, polyacetylenes, polyphenylene sulfide, polythiophene, polyfluorene, polypyrene, polyazulene, polynaphthalene, polycarbazole, polyindole, polyazepine, poly (3, 4-ethylenedioxythiophene) (PEDOT), poly (p-phenylene sulfide) (PPS), poly(p-phenylene vinylene), poly(fluorenes)s, polyphenylenes, polypyrenes, polyazulenes, polynaphthalenes, polyanilines, polyazepines, polyindoles, polycarbazoles, poly(pyrrole)s, poly(thiophene)s, poly (p-phenylene vinylene) (PPV), and mixtures thereof.

The conductive element can be cross-linked with other components present in the composition (i.e., the mixture). For example, the conductive element can be cross-linked with the antimicrobial agent, the proteinaceous material, or itself. In some embodiments, the conductive element is cross-linked with another component of the mixture or by a cross-linking agent. In some embodiments of any one of the aspects described herein, the conductive element is cross-linked with another component of the mixture or to itself by a cross-linking agent by a cross-linking agent selected from genipin, polyethylene glycol, and glutaraldehyde. For example, the conductive element is cross-linked with another component of the mixture or to itself by genipin.

The ratio of the conductive element to the cross-linking agent can be from about 100:1 to about 1:1 (w/w). In some embodiments of any one of the aspects described herein, the ratio of the conductive element to the cross-linker is from about 100:1 to about 10:1 (w/w). For example, the ratio of the proteinaceous material to the cross-linker can be from about 90:1 to about 20:1, about 80:1 to about 30:1, about 70:1 to about 40:1, or about 60:1 to about 50:1 (w/w). In some embodiments, the w/w ratio of the conductive element to cross-linker is about 100:1, or about 95:1, or about 90:1, or about 85:1, or about 80:1, about 75:1, or about 70:1, or about 65:1, or about 60:1, about 55:1, or about 50:1, or about 45:1, or about 40:1, about 35:1, or about 30:1, or about 25:1, or about 20:1, or about 15:1, or about 10:1, or about 5:1 or about 1:1.

The amount of the conductive element in the composition can range from about 1 mg/ml to about 20 mg/ml. For example, the amount of the conductive element in the composition can be about 1 mg/ml, about 1.5 mg/ml, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, about 10 mg/ml, about 10.5 mg/ml, about 11 mg/ml, about 11.5 mg/ml, about 12 mg/ml, about 12.5 mg/ml, about 13 mg/ml, about 13.5 mg/ml, about 14 mg/ml, about 14.5 mg/ml, about 15 mg/ml, about 15.5 mg/ml, about 16 mg/ml, about 16.5 mg/ml, about 17 mg/ml, about 17.5 mg/ml, about 18 mg/ml, about 18.5 mg/ml, about 19 mg/ml, about 19.5 mg/ml, or about 20 mg/ml. In some embodiments of any one of the aspects described herein, the amount of the conductive element in the composition is from about 2 mg/ml to about 18 mg/ml, about 3 mg/ml to about 17 mg/ml, about 4 mg/ml to about 16 mg/ml. For example, the amount of the conductive element in the composition is from about 5 mg/ml to about 15 mg/ml. In some embodiments, amount of the conductive element in the composition is from about 5 mg/ml to about 10 mg/ml.

### Cross-linker

The components of the mixture, e.g., the proteinaceous material, the conductive element, and the anti-microbial agent can be cross-linked to each other or to themselves. As used herein, the term “cross-linked” is intended to refer to two or more molecules covalently bonded together. Cross-linking can be intermolecular, i.e., between different components/molecules, or intramolecular, e.g., between the same component/molecule. Further, covalent bonding between two cross-linkable components/molecules can be direct, in which case an atom in one component/molecule is directly bound to an atom in the other component/molecule, or it can be indirect, through a linking group/agent. The term “cross-linkable” refers to a component or molecule that is capable of undergoing reaction to form a cross-linked composition.

The terms “cross-linking agent”, “cross-linker”, and the like are used interchangeably herein and refer to a compound or molecule that can create a covalent linkage between two cross-linkable components/molecules. Generally, a cross-linking agent contains at least two reactive functional groups that generate covalent bonds between two or more molecules. Cross-linking agents can be homobifunctional (i.e., having two identical reactive ends) or heterobifunctional (i.e., having two different reactive ends). Suitable cross-linking agents include, but are not limited to, genipin; polyethylene glycol (PEG); glutaraldehyde; nordihydroguaiaretic acid (NDGA); 3,4-dihydroxyphenylalanine; 1,2-benzenediol; 2,3-dihydroxynaphthalene; 1,3-benzenediol; adrenalone; catechin; nitrocatechol; 3,4-dihydroxybenzaldehyde; 3,4-dihydroxybenzoic acid; deoxyepinephrine; dobutamine; dopamine; dopexamine; epinephrine; nordefrin; 3-pentadecylcatechol; carbodiimides (e.g., 1-ethyl-3-(3-dimethylaminopropyl) carbodiimide hydrochloride (EDC)); formaldehyde; tannic acid; isocyanates; epichlorohydrin; oxalic acid; malonic acid; succinic acid; glutaric acid; adipic acid; pimelic acid; fumaric acid; maleic acid; malic acid; tartrate; bisepoxides; divinyl sulfone and derivatives (e.g., divinyl sulfone (DVS)); butanediol diglycidyl ether (BDDE); dimethyl adipimidate (DMA), dimethyl suberimidate (DMS), Bissulfosuccinimidyl suberate, formaldehyde, p-azidobenzoyl hydrazide; n-5-azido-2-nitrobenzoyloxysuccinimide; n-[4-(p-azidosalicylamido)butyl]-3′-(2′-pyridyldithio) propionamide; p-azidophenyl glyoxal monohydrate; bis [b-(4-azidosalicylamido)ethyl]disulfide; bis [2-(succinimidooxycarbonyloxy)ethyl] sulfone; 1,4-di [3′-(2′-pyridyldithio)propionamido] butane; dithiobis(succinimidyl propionate); disuccinimidyl suberate; disuccinimidyl tartrate; 3,3′-dithiobis(sulfosuccinimidyl propionate); 3,3′-dithiobis(sulfosuccinimidyl propionate) 1-ethyl-3-[3-dimethylaminopropyl]carbodiimide hydrochloride; ethyleneglycol bis(succinimidyl succinate); N-(E-maleimidocaproic acid hydrazide); [N-(E-maleimidocaproyloxy)-succinimide ester]; N-maleimidobutyryloxysuccinimide ester; hydroxylamine. HCl; maleimide-PEG-succinimidyl carboxy methyl; m-maleimidobenzoyl-N-hydroxysuccinimide Ester; N-hydroxysuccinimidyl-4-azidosalicylic acid; N-(p-maleimidophenyl isocyanate); N-succinimidyl(4-iodoacetyl) aminobenzoate; succinimidyl-4-(N-maleimidomethyl)cyclohexane-1-carboxylate; succinimidyl 4-(p-maleimidophenyl) butyrate; sulfo-disulfosuccinimidyl tartrate; [N-(E-maleimidocaproyloxy)-sulfo succinimide ester; N-Maleimidobutyryloxysulfosuccinimide ester; N-hydroxysulfosuccinimidyl-4-azidobenzoate; m-Maleimidobenzoyl-N-hydroxysulfo succinimide ester; sulfosuccinimidyl (4-azidophenyl)-1,3 dithio propionate; sulfosuccinimidyl 2-(m-azido-o-nitrobenzamido)-ethyl-1,3′-dithio propionate; sulfosuccinimidyl 6-(4′-azido-2′-nitrophenylamino) hexanoate; sulfosuccinimidyl-2-(p-azidosalicylamido)ethyl-1,3-dithiopropionate; N-(Sulfosuccinimidyl(4-iodoacetyl) amino benzoate); sulfosuccinimidyl-4-(N-maleimidomethyl)cyclohexane-1-carboxylate; sulfosuccinimidyl 4-(p-maleimidophenyl) butyrate; poly (ethylene glycol) diglycidyl ether (PEGDE); poly (propylene glycol) diglycidyl ether (PPGDE); and the like. In some embodiments of any one of the aspects described herein, the cross-linking agent is genipin, polyethylene glycol (PEG), or glutaraldehyde. In some preferred embodiments of any one of the aspects described herein the cross-linking agent is genipin.

### Target Binding Molecule

In some embodiments of any one of the aspects described herein, the composition, e.g., mixture described herein further comprises target binding molecule. As used herein, the term “target binding ligand” refers to a molecule that binds to or interacts with a target molecule. In other words, a target binding ligand is a molecule that is capable of binding with a target molecule. The targeting binding ligand can be a natural or synthetic molecule (e.g., a molecular receptor) that binds to a target molecule. Exemplary target binding ligands include, but are not limited to, a receptor, a ligand for a receptor, an antibody, an antigen binding fragment of an antibody, an antigen, an enzyme, or a nucleic acid. The target binding ligand is also referred to as a “capture agent” or “capture molecule” herein.

In some embodiments of any one of the aspects described herein, the binding of the target binding ligand to the target molecule is a specific binding such that it is selective to that target above non-targets. For example the dissociation constant between the target binding ligand and target molecule is at least about 200 nM, alternatively at least about 150 nM, alternatively at least about 100 nM, alternatively at least about 60 nM, alternatively at least about 50 nM, alternatively at least about 40 nM, alternatively at least about 30 nM, alternatively at least about 20 nM, alternatively at least about 10 nM, alternatively at least about 8 nM, alternatively at least about 6 nM, alternatively at least about 4 nM, alternatively at least about 2 nM, alternatively at least about 1 nM, or greater. In certain embodiments, the specific binding refers to binding where the target binding ligand binds to its target molecule without substantially binding to any other species in the sample/test solution.

By way of non-limiting examples, a target binding ligand can be selected from antibodies, adnectins, ankyrins, other antibody mimetics and other protein scaffolds, aptamers, nucleic acid (e.g., an RNA or DNA aptamer), protein, peptide, binding partner, oligosaccharides, polysaccharides, lipopolysaccharides, cellular metabolites, cells, viruses, subcellular particles, haptens, pharmacologically active substances, alkaloids, steroids, vitamins, amino acids, avimers, peptidomimetics, hormone receptors, cytokine receptors, synthetic receptors, sugars or molecularly imprinted polymer. The target binding ligand can be selective to a specific target or class of targets such as toxins and biomolecules. For example, the target can be ions, molecules, oligomers, polymers, proteins, peptides, nucleic acids, toxins, biological threat agents such as spore, viral, cellular and protein toxins, carbohydrates (e.g., mono saccharides, disaccharides, oligosaccharides, polyols, and polysaccharides) and combinations of these (e.g., copolymers including these).

In some embodiments of any one of the aspects described herein, the target binding ligand is an antibody or antigen binding fragment thereof. As used herein, the terms “antibody” and “antibodies” include polyclonal antibodies, monoclonal antibodies, humanized or chimeric antibodies, single chain Fv antibody fragments, Fab fragments, and F(ab)₂ fragments. Antibodies having specific binding affinity for a target of interest (e.g., an antigen) can be produced through standard methods. As used herein, the terms “antibody” and “antibodies” refer to intact antibody, or a binding fragment thereof that competes with the intact antibody for specific binding and includes chimeric, humanized, fully human, and bispecific antibodies. In some embodiments, binding fragments are produced by recombinant DNA techniques. In additional embodiments, binding fragments are produced by enzymatic or chemical cleavage of intact antibodies. Binding fragments include, but are not limited to, Fab, Fab′, F(ab′)₂, Fv, and single-chain antibodies.

### Additional Components

In some embodiments, the composition, i.e., the mixture further comprises one or more polymers. Exemplary polymers include, but are not limited to, polyethylene glycol (PEG), alginate, polytetrafluoroethylene (PTFE), polyacetylene (PA), polyaniline (PANI), polypyrrole (PPy), polythiophene (PTH), poly(para-phenylene) (PPP), poly(phenylenevinylene) (PPV), and polyfuran (PF). In another example, in some implementations, the polymer is a natural polymer such as cellulose, natural silk, cotton, or natural rubbers. In some implementations, the polymer is a synthetic polymer, such as nylon, epoxies, polyethylene (e.g. HDPE and LDPE), polypropylene, polybutadiene, polyethylene terephthalate (PET), polycarbonate, polyurethane, fluorinated polymers (e.g. TEFLON®), polystyrene (e.g. Styrofoam), sulfonated polystyrene, aramide (e.g. KEVLAR®), poly acrylonitrile, poly vinyl acetate, poly vinyl chloride (PVC), poly methyl methacrylate (PMMA), Polyhydroxyethylmethacrylate (PolyHEMA), poly ethers, poly lactic acid, and copolymers and blends of these. In some implementations, the polymer is an ionic polymer, such as a cationic or anionic polymer. In some embodiments, the mixture comprises a polymer selected from the group consisting of polyethylene glycol (PEG), alginate, polytetrafluoroethylene (PTFE), polyacetylene (PA), polyaniline (PANI), polypyrrole (PPy), polythiophene (PTH), poly(para-phenylene) (PPP), poly(phenylenevinylene) (PPV), and polyfuran (PF).

In some embodiments, the composition, i.e., the mixture further comprises a drug eluting component. For example, the mixture further comprises a therapeutic in addition to the anti-microbial agent. Exemplary therapeutic agents include, but are not limited to, anti-inflammatory agents, anti-cancer agents, anti-proliferatives, anti-migratory agents, anti-fibrotic agents, proapoptotics, anti-neoplastics, immuno-suppressants, and hormones.

In some embodiments, the therapeutic agent is an anti-inflammatory agent. As used herein the term “anti-inflammatory agent” refers to a compound (including its analogs, derivatives, prodrugs and pharmaceutically salts) which can be used to treat inflammation or an inflammation related disease or disorder. Exemplary anti-inflammatory agents include, but are not limited to, the known steroidal anti-inflammatory and non-steroidal anti-inflammatory drugs (NSAIDs). Exemplary steroidal anti-inflammatory agents include but are not limited to 21-acetoxypregnenolone, alclometasone, algestone, amcinonide, beclomethasone, betamethasone, budesonide, chloroprednisone, clobetasol, clobetansone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furcate, paramethosone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, and analogues and derivatives thereof. Exemplary nonsteroidal anti-inflammatory agents include but are not limited to COX inhibitors (COX-1 or COX nonspecific inhibitors) and selective COX-2 inhibitors. Exemplary COX inhibitors include but are not limited to salicylic acid derivatives such as aspirin, sodium salicylate, choline magnesium trisalicylate, salicylate, diflunisal, sulfasalazine and olsalazine; para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin; anthranilic acids (fenamates) such as mefenamic acid and meloxicam; enolic acids such as the oxicams (piroxicam, meloxicam); alkanones such as nabumetone; and analogues and derivatives thereof. Exemplary COX-2 inhibitors include but are not limited to diarylsubstituted furanones such as refecoxib; diarylsubstituted pyrazoles such as celecoxib; indole acetic acids such as etodolac and sulfonanilides such as nimesulide; and analogues and derivatives thereof. Additional anti-inflammatory agents include, but are not limited to sirolimus, everolimus, biolimus (A9), zotarolimus (ABT-578), tacrolimus, pimecrolimus, and genistein.

In some embodiments, the mixture is a homogenous mixture.

In some embodiments, the composition has antimicrobial activity.

### Antifouling Surface

Without wishing to be bound by a theory, the composition, i.e., the mixture described herein has antifouling properties. As used herein, the term “antifouling” refers to the effect of preventing, reducing and/or eliminating fouling, i.e., preventing, reducing and/or eliminating the aggregation of molecules such as biomolecules on a surface such that the surface maintains its initial physical and/or chemical properties (e.g. conductivity). Thus, in some embodiments, the composition is antifouling. The composition can be coated on a surface to impart antifouling properties to the surface. Accordingly, in another aspect provided herein is a surface with antifouling properties. For example, the surface with the antifouling properties comprises a composition described herein.

In the context of this specification, the term “coated” means that a layer of is present on a surface. For example, a layer of antifouling layer on a surface or a layer of probe on the antifouling layer. The amount of the probe used to coat the antifouling layer can vary with a number of factors such as surface area, coating density, types of probe, and binding performance.

Without wishing to be bound by a theory, the composition described herein is biocompatible. The term “biocompatible” refers to a material's ability to perform its intended function, with a desired degree of incorporation in a host, without eliciting any undesirable local or systemic effects in that host. Accordingly, in some embodiments, the coated surface is a surface of a medical device. As used herein, a “medical device” refers to a non-naturally occurring object that is inserted or implanted in a subject or applied to a surface of a subject. Exemplary medical devices include, but are not limited to, fibers (wound dressings, bandages, gauze, tape, pads, sponges, including woven and non-woven sponges and those designed specifically for dental or ophthalmic surgeries), surgical, medical or dental instruments, blood oxygenators, ventilators, pumps, drug delivery devices, tubing, wiring, electrodes, contraceptive devices, feminine hygiene products, endoscopes, grafts (including small diameter<6 mm), stents (including coronary, uretheral, renal, biliary, colorectal, esophageal, pulmonary, urethral, and vascular), stent grafts (including abdominal, thoracic, and peripheral vascular), pacemakers, implantable cardioverter-defibrillators, cardiac resynchronization therapy devices, cardiovascular device leads, ventricular assist devices and drivelines, heart valves, vena cava filters, endovascular coils, catheters (including central venous, peripheral central, midline, peripheral, tunneled, dialysis access, urinary, neurological, peritoneal, intra-aortic balloon pump, angioplasty balloon, diagnostic, interventional, drug delivery, etc.), catheter connectors and valves (including needleless connectors), intravenous delivery lines and manifolds, shunts, wound drains (internal or external including ventricular, ventriculoperitoneal, and lumboperitoneal), dialysis membranes, infusion ports, cochlear implants, endotracheal tubes, tracheostomy tubes, ventilator breathing tubes and circuits, guide wires, fluid collection bags, drug delivery bags and tubing, implantable sensors (e.g., intravascular, transdermal, intracranial), ophthalmic devices including contact lenses, orthopedic devices (including hip implants, knee implants, shoulder implants, spinal implants (including cervical plates systems, pedicle screw systems, interbody fusion devices, artificial disks, and other motion preservation devices), screws, plates, rivets, rods, intramedullary nails, bone cements, artificial tendons, and other prosthetics or fracture repair devices), dental implants, periodontal implants, breast implants, penile implants, maxillofacial implants, cosmetic implants, valves, appliances, scaffolding, suturing material, needles, hernia repair meshes, tension-free vaginal tape and vaginal slings, prosthetic neurological devices, tissue regeneration or cell culture devices, or other medical devices used within or in contact with the body or any portion of any of these.

In some embodiments, the coating layer is directly or indirectly connected to an electrode.

### Electrode

In another aspect, provided herein is an electrode comprising a conductive substrate and an antifouling coating layer on at least a portion of a surface of the conductive substrate, wherein the antifouling coating layer comprises a composition described herein.

As used herein an “electrode” is a conductor through which current enters or leaves a medium, where the medium is nonmetallic (i.e., it emits or collects electrons or electron “holes”). For example, the medium can be a complex matrix (e.g., blood or serum). The electrode can be inserted into/onto a tissue such as mammalian tissue and be contacted with tissue and/or fluids therein/thereon. The electrode can be large (e.g., with a working surface area of greater than 1 cm ², greater than 10 cm², greater than 100 cm²) or the electrode can be small (e.g., with a working surface area of less than 1 cm², less than 1 mm², less than 100 μm², less than 10 μm², less than 1 μm²). The working surface area is the area in contact with the medium and wherein current enters or leaves the medium.

The conductive substrate can be in any form having a surface that can be coated. For example, the conductive substrate can be included in the form of a conductive particle, a conductive nano-particle, a conductive micro-particle, a conductive nano-fiber, a conductive micro-fiber, a conductive flake, a conductive chip, a conductive crystal, a conductive porous substrate, a conductive wafer, a conductive wire, a conductive nano-wire, a conductive micro-wire, a conductive channel, a conductive nano-channel, a conductive micro-channel, a conductive rod, a conductive nano-rod, a conductive micro-rod, a conductive foil, a conductive sheet, a conductive web, or combination of these forms. In some implementations, the conductive substrate is part of a microfluidic device, such as a channel or chamber therein.

Metal patterning techniques, such as standard printed circuit board (PCB) technology, offer a number of versatile fabrication options such as (i) track size and spacing less than 100 μm; (ii) high purity electrolytic gold plating several microns thick suitable for electrochemistry and surface modification chemistries; (iii) ease of small scale prototyping in standard laboratory settings; and (iv) large scale mass manufacturing capabilities at a fraction of the cost of high-end microarrays. In some embodiments, electrodes as disclosed herein may be fabricated using PCB technology.

In some embodiments, the electrodes are mass fabricated onto non-electrically conductive surfaces such as plastic substrates using inexpensive standard technology such as printed circuit board (PCB) technology, roll-to-roll laser ablation or evaporation. Exemplary non-electrically conductive surfaces include plastic, poly(carbonate) (PC), poly(methyl methacrylate) (PMMA), cyclic olefin polymers (COP) or cyclic olefin copolymers (COC), SU-8, parylene, silicon nitride, kapton, styrene-ethylene-butylene-styrene (SEBS), poly-dimethysiloxane (PDMS), polyimide, silicon dioxide, and any combination thereof.

In some embodiments, the electrode is a planar or a 3-dimensional electrode. As used herein, a planar electrode electrically interacts with an electroactive species or mediator on a 2-dimensional surface. As used herein, a 3-dimensional electrode is an electrode displaying a very high surface area per unit volume, caused by no planarity. Without being bound by theory, this provides high turbulence at their interface with an electroactive species or mediator, enhancing the mass transfer process of the electroactive species towards the electrode surface. These characteristics strongly improve the electrochemical reaction rate.

In some embodiments the electrode is “Multiplexed” such that it is configured for a multiplexed assay. As used herein a “multiplexed” assay can be used to simultaneously measure multiple analytes or signals such as two or more (e.g., 3 or more, 5 or more, 10 or more, 50 or more, 100 or more, 1000 or more) during a single run or cycle of the assay. The electrode can therefore be configured as an array of electrodes, microelectrodes or electrochemical sensors each of which can be independently electrically attached to a circuit for monitoring the electrical signals. For example, the array of electrodes can be disposed at the bottom, sides or top of a multiwell plate (e.g., microwell plate) arrayed on a flat surface such as a semiconductor chip (e.g., a sensor array chip) or form part of a multielectrode array (e.g., for connection of neurons to electronic circuitry). In some embodiments, the compositions as described herein, can coat more than one sensor since the coating will not conduct between the sensors due to the anisotropy of the conduction, therefore an array of conductors, sensors or electrodes can be coated forming a multiplexed electrode.

Electrodes can include materials with metallic conduction and semiconductors. For example, electrodes can include metals, metal alloys, semiconductors, doped materials, conducting ceramics and conducting polymers. Without limitation, electrode materials can include carbon (e.g., graphite, glassy carbon, conductive polymers), copper, titanium, brass, mercury, silver, platinum, palladium, gold, rhodium, zinc, lead, tin, iron, Indium Tin Oxide (ITO), aluminum, stainless steel, tungsten, nickel, zirconium, niobium, tantalum, chromium, molybdenum, manganese, rhenium, ruthenium, rhodium, iridium, silicon, doped silicon, II-VI semiconductors (e.g., ZnO, ZnS, CdSe), III-V semiconductors such as (e, g., GaAs, InSb), ceramics (e.g., TiO₂, Fe₃O₄, MgCr₂O₄), and conductive polymers (e.g., poly(acetylene)s, poly(p-phenylene vinylene), poly(fluorenes)s, polyphenylenes, polypyrenes, polyazulenes, polynaphthalenes, polyanilines, polyazepines, polyindoles, polycarbazoles, poly(pyrrole)s, poly(thiophene)s, poly(3,4-ethylenedioxythiophene)), polyimide, parylene, benzocyclobutene, and combinations, mixtures and alloys of these.

In some embodiments, the conductive substrate includes a metal, a metalloid, a conducting polymer, a conducting glassy material, a conducting amorphous material, a conducting biological membrane, a conducting carbon based material, or any combination of these.

In some embodiments, the conductive substrate includes gold. In some embodiments, the conductive substrate includes a silica based glass (e.g., pure silica or mixtures such as borosilicate glass). In some embodiments, the conductive substrate includes graphite, diamond, glassy carbon, or carbon nano-tubes (CNTs). In some implementations, the conductive substrate is a chip including gold and a silica based glass.

In some embodiments of any one of the aspects described herein, the conductive substrate is a flexible substrate. For example, the conductive substrate comprises a flexible material. Exemplary materials for the flexible substrate include, but are not limited to, polyethylene terephthalate, polyethylene naphathalate, polyimides, polymeric hydrocarbons, celluloses, plastics, polycarbonates, polystyrenes, and any combination thereof.

Electrodes can also include insulating components such as insulators for electrical and mechanical protection, imparting rigidity and electrical isolation to parts of the electrode.

In some embodiments, the electrode can be part of an electrochemical cell. For example, the electrode is a working electrode and the electrochemical cell can include a counter electrode and reference electrode.

Electrochemical methods are methods that rely on a change in the potential, charge or current to characterize the analyte's chemical reactivity. Some examples include potentiometry, controlled current coulometry, controlled-potential coulometry, amperometry, stripping voltammetry, hydrodynamic voltammetry, polarography, stationary electrode voltammetry, pulsed polarography, electrochemical impedance spectroscopy and cyclic voltammetry. The signals are detected using an electrode or electrochemical sensors coupled to circuits and systems for collection, manipulation and analysis of the signals.

In some embodiments, the antifouling coating layer of the electrode is adapted for contact with an analyte or a sample comprising an analyte. The antifouling coating can allow analyte to flow through the pores and be detected, for example, by binding to a capture molecule such as an antibody, DNA strand, or aptamer. In some implementations, the coatings can be patterned as a conductive wire or a dielectric/insulating surface. Some implementations include coating microfluidic chips, lab-on-a-chip, and organs on a chip. In some implementations, the coatings can be used in nano-gap and micro-gap devices. For example, these devices include nano-gap electrodes, nanostructured-based electrical biosensors, and nano-gap dielectric biosensor for label free DNA hybridization detection. The coatings can be applied, for example, to the gap between electrodes in the device and thereby protect the surfaces of the gap from fouling. In some implementations, the coating is a cross-linked and porous gel, and the gap is completely or mostly filled in. Analyte can flow through the pores and be detected, for example, by binding to a capture molecule such as an antibody, DNA strand, or aptamer.

In some embodiments, the antifouling coating layer may have a thickness of from about 0.1 nm to about 10 μm, or from about 1 nm to about 5 μm, or from about 10 nm to about 1 μm, or from about 100 nm to about 0.5 μm, or from about 150 nm to about 0.25 μm, or about 200 nm. In some embodiments, the antifouling coating layer has a thickness of about 0.1 nm, about 0.5 nm, about 1 nm, about 10 nm, about 50 nm, about 100 nm, about 200 nm, about 300 nm, about 400 nm, about 500 nm, about 600 nm, about 700 nm, about 800 nm, about 900 nm, about 1 μm, about 2 μm, about 3 μm, about 4 μm, about 5 μm, about 6 μm, about 7 μm, about 8 μm, about 9 μm, or about 10 μm.

In some embodiments of any one of the aspects described herein, the electrode is a gate electrode of a Field Effect Transistor (FET)

### Organic Field Effect Transistor

In another aspect provided herein is an organic field effect transistor (OFET). Generally, the organic field effect transistor comprises: (a) a source electrode; (b) a drain electrode; (c) an organic conducting or semiconducting layer connecting the source electrode to the drain electrode; and (d) an antifouling coating layer on at least a portion of the surface of the organic conducting or semiconducting layer, and wherein the antifouling coating layer comprises a composition as described herein.

In the context of this specification, the term “transistor” refers to a device capable of converting physical quantities into an electrical signal. Preferably, the device is a semiconductor. Preferably, the physical quantities are physical quantities of a chemical produced by an assay. The assay is optionally enzymatic. The assay is optionally a molecular diagnostic and/or involved antibody binding. The assay may be an ELISA assay.

The source and/or drain electrodes can comprise, consist, or consist essentially of an ohmic material. Exemplary ohmic materials include, but are not limited to, as metals (e.g., gold or silver) or metal oxides or graphene. Preferably the source and/or drain electrodes are tin oxide. Preferably the source and/or drain electrodes are indium tin oxide (ITO), for example pre-patterned ITO. Preferably each of said source and drain electrodes consist of an ohmic material. In at least one embodiment, source electrode and drain electrode each comprise, consist, or consist essentially of ITO, for example pre-patterned ITO. In alternate preferred embodiments, the ohmic conductor is a metal based ink such as silver based ink.

In some embodiments, the organic conducting or semiconducting layer of the organic field transistor may have a thickness of from about 0.1 nm to about 10 μm, or from about 1 nm to about 5 μm, or from about 10 nm to about 1 μm, or from about 100 nm to about 0.5 μm, or from about 150 nm to about 0.25 μm, or about 200 nm. In some embodiments, the antifouling coating layer has a thickness of about 0.1 nm, about 0.5 nm, about 1 nm, about 10 nm, about 50 nm, about 100 nm, about 200 nm, about 300 nm, about 400 nm, about 500 nm, about 600 nm, about 700 nm, about 800 nm, about 900 nm, about 1 μm, about 2 μm, about 3 μm, about 4 μm, about 5 μm, about 6 μm, about 7 μm, about 8 μm, about 9 μm, or about 10 μm.

In some embodiments, the antifouling layer of the organic field transistor may have a thickness of from about 0.1 nm to about 10 μm, or from about 1 nm to about 5 μm, or from about 10 nm to about 1 μm, or from about 100 nm to about 0.5 μm, or from about 150 nm to about 0.25 μm, or about 200 nm. In some embodiments, the antifouling coating layer has a thickness of about 0.1 nm, about 0.5 nm, about 1 nm, about 10 nm, about 50 nm, about 100 nm, about 200 nm, about 300 nm, about 400 nm, about 500 nm, about 600 nm, about 700 nm, about 800 nm, about 900 nm, about 1 μm, about 2 μm, about 3 μm, about 4 μm, about 5 μm, about 6 μm, about 7 μm, about 8 μm, about 9 μm, or about 10 μm.

In at least one embodiment, the organic conducting or semiconducting layer consists of one organic semiconductor.

The organic conducting or semiconducting layer may be disposed above and in between the source electrode and the drain electrode. In an embodiment, the organic conducting or semiconducting layer is connected to and between a source electrode and a drain electrode. The organic conducting or semiconducting layer may be disposed above and in between the source electrode and the drain electrode, and in direct contact with, the source electrode and the drain electrode.

The organic conducting or semiconducting layer is preferably in contact with, the source electrode and the drain electrode. At least part of the organic conducting or semiconducting layer is disposed above the source electrode and the drain electrode. Preferably a majority of the organic conducting or semiconducting layer is disposed above the source electrode and the drain electrode.

The organic conducting or semiconducting layer is configured to enable flow of electrical current between the source electrode and the drain electrode as a result of the generation of charge carriers.

The organic conducting or semiconducting layer includes one or more organic compounds having conducting or semiconducting properties. Exemplary organic compounds having conducting or semiconducting properties include, but are not limited to, polyacetylenes, porphyrins, phthalocyanins, fullerenes, polyparaphenylenes, polyphenylenevinylenes, polyfluorenes, polythiophenes, polypyrroles, polypyridines, polycarbazoles, polypyridinevinylenes, polyarylvinylenes, poly (p-phenylmethylvinylenes), including derivatives and co-polymers thereof, and further including combinations thereof. In some embodiments of any one of the aspects described herein, the organic conducting or semiconducting layer comprises an organic compound having conducting or semiconducting properties and selected from the group consisting of: poly(9,9-dioctylfluorene-2,7-diyl-co-bis-N, N-(4-butylphenyl)-bis-N, N-phenyl-1,4-phenylenediamine), poly(9,9-dioctylfluorene-2,7-diyl-co-benzothiadiazole), poly(3-hexylthiophene), perfluoro-3,6-dioxa-4-methyl-7-octenesulfonic acid copolymer, tetrafluoroethylene-perfluoro-3,6-dioxa-4-methyl-7-octenesulfonic acid copolymer (e.g. nafion), poly(9,9-dioctylfluorene-2,7-diyl-co-bis-N, N-(4-butylphenyl)-bis-N, N-phenyl-1,4-phenylenediamine), poly(9,9-dioctylfluorene-2,7-diyl-co-benzothiadiazole), (6,6)-phenyl-C61-butyric acid methyl ester, poly(2-methoxy-5-(2′-ethyl-hexyloxy)-1,4-phenylene vinylene), poly(4-vinylphenol) (PVP), and copolymers and combinations thereof. In some embodiments of any one of the aspects described herein, the organic conducting or semiconducting layer includes, consists of, or consists essentially of poly(3-hexyl-thiophene) (P3HT).

In some implementations, the organic field effect transistor further comprises a conducting polymer layer between the organic conducting or semiconducting layer and the antifouling layer. In some implementations, the conducting polymer layer comprises one or more of a e.g. fluorinated polymers e.g. poly methyl methacrylate (PMMA), Polyhydroxyethylmethacrylate (PolyHEMA), polyacetylenes, porphyrins, phthalocyanins, fullerenes, polyparaphenylenes, polyphenylenevinylenes, polyfluorenes, polythiophenes, polypyrroles, polypyridines, polycarbazoles, polypyridinevinylenes, polyarylvinylenes, poly(p-phenylmethylvinylenes), including derivatives and co-polymers thereof, and further including mixtures thereof and copolymers and blends of these.

In some embodiments of any one of the aspects described herein, the conducting polymer layer comprises one or more of: poly(3-hexylthiophene), perfluoro-3,6-dioxa-4-methyl-7-octenesulfonic acid copolymer, tetrafluoroethylene-perfluoro-3,6-dioxa-4-methyl-7-octenesulfonic acid copolymer (e.g. nafion), poly(9,9-dioctylfluorene-2,7-diyl-co-bis-N, N-(4-butylphenyl)-bis-N, N-phenyl-1,4-phenylenediamine), poly(9,9-dioctylfluorene-2,7-diyl-co-benzothiadiazole), (6,6)-phenyl-C61-butyric acid methyl ester, poly(2-methoxy-5-(2′-ethyl-hexyloxy)-1,4-phenylene vinylene), poly(4-vinylphenol) (PVP), and copolymers thereof, and any combination thereof. In some embodiments, the conducting polymer layer comprises poly(3-hexyl-thiophene) or nafion.

In some embodiments of any one of the aspects described herein, the conducting polymer is a gating layer between the anti-fouling layer and the organic or semiconducting layer.

In some embodiments, the conducting polymer layer of the organic field transistor may have a thickness of from about 0.1 nm to about 10 μm, or from about 1 nm to about 5 μm, or from about 10 nm to about 1 μm, or from about 100 nm to about 0.5 μm, or from about 150 nm to about 0.25 μm, or about 200 nm. In some embodiments, the antifouling coating layer has a thickness of 0.1 about nm, about 0.5 nm, about 1 nm, about 10 nm, about 50 nm, about 100 nm, about 200 nm, about 300 nm, about 400 nm, about 500 nm, about 600 nm, about 700 nm, about 800 nm, about 900 nm, about 1 μm, about 2 μm, about 3 μm, about 4 μm, about 5 μm, about 6 μm, about 7 μm, about 8 μm, about 9 μm, or about 10 μm.

In some embodiments, the organic field effect transistor further comprises a gate electrode in contact with the antifouling coating layer or the organic conducting or semiconducting layer.

In some embodiments, the antifouling coating layer is configured to be connected to an ohmic conductor for applying a gate voltage to said organic conducting or semiconducting layer (e.g., via antifouling layer). Optionally, the antifouling coating layer is connected to the ohmic conductor. Optionally, the ohmic conductor is in contact with the antifouling coating layer and/or the organic conducting or semiconducting layer.

In some embodiments, the ohmic conductor comprises, consists, or consists essentially of an ohmic material, such as metals (e.g., gold or silver) or metal oxides or graphene. Preferably the ohmic conductor is tin oxide. Preferably the ohmic conductor is indium tin oxide (ITO), for example pre-patterned ITO. In alternate preferred embodiments, the ohmic conductor is a metal based ink such as silver based ink. In some embodiments the silver based ink comprises silver nanoparticles (AgNPs).

In some embodiments, the ohmic conductor is a gate electrode The ohmic conductor or gate electrode is optionally laterally offset from the source and drain electrodes, as is part of the polymer layer. When the gate voltage is applied via the ohmic conductor/gate electrode to the polymer gating layer, a substantial electric field results in a vertical plane (i.e., in a plane perpendicular to the top surface of the semiconductor layer).

In some embodiments, the organic field effect transistor further comprises a substrate. Optionally, the substrate is on the opposite side of the OFET to the antifouling layer.

In some embodiments, the organic field effect transistor is at least a partially printed OFET. For example, the organic conducting/seminconducting layer, antifouling layer and optionally the conducting polymer layer of the OFET are printed on a substrate. In some embodiments, the antifouling layer and optionally the conducting polymer layer are printed onto the the organic conducting/seminconducting layer. In some embodiments, the electrodes are printed on the substrate, for example, prior to printing of the organic conducting/seminconducting layer, antifouling layer and optionally the conducting polymer layer. Printing methods include gravure, flexographic, screen-printing and doctor blade. Alternatively, non-printing methods known to those skilled in the art may be used, such as drop casting, vapour deposition and sputtering.

In some embodiments, the organic field effect transistor further comprises a dielectric layer intermediate the conducting polymer layer and the organic condiucting/semiconductor layer.

The dielectric layer may comprise, consist of, or consist essentially of an organic dielectric material. Preferably, the organic dielectric material has a conductivity to protons that is greater than the conductivity of said organic conducting/semiconducting layer. Preferably the dielectric layer intermediate said conducting polymer layer and said organic conducting/semiconducting layer is a homogenous layer. Preferably the organic dielectric material is a hygroscopic insulator, such as for example polyvinyl phenols. More specifically, the dielectric layer may comprise, consist of, or consist essentially of, poly(4-vinylphenol). Alternative dielectric materials that may be used in the devices will be readily apparent to those skilled in the art. Non-limiting examples include polyimide and poly(methyl methacrylate) (PMMA). In alternative embodiments the dielectric layer may comprise a doped dielectric material, for example lithium perchlorate doped poly(4-vinylpyridine).

The dielectric layer may have a thickness between about 50 nm and 750 nm, or between about 300 nm and about 500 nm, or about 400 nm.

The dielectric layer may be in contact with the organic conducting/semiconducting layer. Additionally, or alternatively the dielectric layer may be in contact with the conducting polymer layer. The dielectric layer may be disposed above the organic conducting/semiconducting layer. The dielectric layer may be disposed above, and in direct contact with, the organic conducting/semiconducting layer. The dielectric layer may be disposed below the conducting polymer layer. The dielectric layer may be disposed below, and in direct contact with, the conducting polymer layer.

The gate electrode/ohmic conductor ma y be disposed above the dielectric layer. The gate electrode may be disposed above, and in direct contact with, the dielectric layer or disposed above the dielectric layer, and in indirect contact with the dielectric layer via the polymer layer and/or the antifouling layer. The gate electrode/ohmic conductor may be offset with respect to the dielectric layer.

In some embodiments, the ohmic conductor is in contact with the dielectric layer and configured to apply a gate voltage to the dielectric layer.

### Analyte Detection

The electrode and the organic field effect transistor described herein can be used for detecting analytes, e.g., in a sample. Accordingly, another aspect provided herein relates to methods of detecting at least one target analyte, including, e.g., at least 2, 3, 4, 5, 6, 7, 8 target analytes or more. Generally, the method comprises contacting a sample suspected of comprising a target analyte with an electrode or organic field effect transistor described herein and detecting the binding of the target analyte with the target binding ligand. The binding may be detected electrochemically.

In some embodiments, the method of detecting a target analyte comprises contacting a sample suspected of comprising a target analyte with an electrode described herein and detecting the binding of the target analyte with the target binding ligand. Optionally, detecting the binding of the target molecule with the target binding ligand comprises applying a voltage to the electrode and measuring the current generated from the electrode.

In some embodiments, the method of detecting a target analyte comprises contacting a sample suspected of comprising a target analyte with an organic field effect transistor described herein and detecting the binding of the target analyte with the target binding ligand. Optionally, detecting the binding of the target molecule with the target binding ligand comprises applying a voltage to the drain electrode or source ohmic conductor and detecting a drain current. Optionally, the source electrode is grounded.

The applied voltage provides a sufficiently strong electric field to liberate H⁺ from H₂O₂, but not strong enough to cause electrolysis of water, as electrolysis of water may lead to a decrease in the signal-to-noise ratio (i.e. below −1.23 V). Typically, the gate voltage and drain voltage applied are between about 0 V and −2 V, e.g. about −1 V. In some embodiments, low voltages can be used including 250 to 500 mV to 300-400 mV. The voltage range can then be −0.25 to −2V or −0.25 to −0.1 V.

When the sensor is an OFET with an organic semiconductor with a conducting layer (e.g., nafion), the voltage applied (e.g., by an ohmic conductor/gate electrode) is at least −0.55 V, −0.6 V, −0.65 V or −0.7V (i.e. −0.55 V to −2 V). −0.6 to −0.8V and −0.65 to −0.75 V are preferred ranges.

The voltage applied by the ohmic conductor/gate electrode is optionally at least 0.7 V.

In some embodiments, the target of the target binding molecule can be redox active (e.g., an electroactive analyte) and is directly detected by an electrode. For example, the target binding molecule facilitates detection of the target analyte by the electrode due to it concentrating the analyte near or at the surface of the electrode where it can be detected directly by electrochemical means. In some embodiments, the electrode is a gate electrode of a Field Effect Transistor (FET) and the change in concentration of the target of the target binding molecule changes the voltage in a range to activate/deactivate the gate.

In some embodiments, the binding of the target analyte to the target binding molecule is detected indirectly by electrochemical means. For example, the target can be detected by binding with a detection agent that catalyzes, directly or indirectly, a redox reaction close to an electrode surface. For example, the target analyte can be contacted with a labeling probe, e.g., a second target binding molecule, wherein the labeling probe comprises a detectable label. Generally, the labeling probe comprises a target binding molecule capable of binding with the target analyte. For example, the labeling probe is a target binding molecule described herein. In some embodiments, the labeling probe is an antibody, antigen binding fragment of an antibody, an antigen, a receptor, a ligand for a receptor, an enzyme, or a nucleic acid.

As used herein, the term “detectable label” refers to a molecule or composition capable of producing a detectable signal indicative of the presence of a target. Exemplary detectable labels include but are not limited to an enzyme, a fluorophore, a chemiluminescent label, colloidal gold, colored glass or plastic (e.g., polystyrene, polypropylene, and latex) beads, a radiolabel, a quantum dot, or any combination thereof.

In some embodiments, the labeling probe contacts the target analyte prior to contacting the sample with the electrode or organic field effect transistor. In some other embodiments, the labeling probe contacts the target analyte after contacting the sample with the electrode or organic field effect transistor. In some embodiments of any one of the aspects described herein, the detectable label deposits a sacrificial redox active molecule on the electrode surface (e.g., on a coating that is on the surface of the electrode) that then is detected electrochemically.

In some embodiments of any one of the aspects described herein, the detectable label comprises an enzyme. Non-limiting examples of enzymes include: a peroxidase, alkaline phosphatase, malate dehydrogenase, staphylococcal nuclease, delta-V-steroid isomerase, yeast alcohol dehydrogenase, alpha-glycerophosphate dehydrogenase, triose phosphate isomerase, horseradish peroxidase (HRP), alkaline phosphatase, asparaginase, glucose oxidase, beta-galactosidase, ribonuclease, urease, catalase, glucose-VI-phosphate dehydrogenase, glucoamylase, tyrosinase, acetylcholinesterase, or any combination thereof. In some preferred embodiments, the enzyme is a peroxidase or alkaline phosphatase.

When the detectable label comprises an enzyme, the method can further comprise contacting the enzyme with a substrate of the enzyme. Exemplary reporter enzyme substrates include, but are not limited to, hydrogen peroxide, carbamide peroxide, nucleotides, oligonucleotides, RNA, DNA, phosphorylated peptides, phosphorylated proteins, phosphorylated small molecules, glucose, phenols, tyrosine, dopamine, catechol, urea, and any combination thereof.

In some embodiments, the reporter enzyme substrate is hydrogen peroxide. In some embodiments the target analyte can be redox active and the enzyme is directly responsible for generation of a charge carrier that is detected by an electrode. For example, the binding of the target analyte to the second target binding molecule facilitates generation of the charge carrier near the antifouling layer surface, the conducting antifouling layer conducts the charge carrier and this impacts the applied voltage and/or current resulting in detection of the target analyte. In one non-limiting example, the organic semiconductor is doped by interaction with said charge carriers to increase an electrical conductivity between the drain electrode and the source electrode.

The charge carrier can be any one or more of the following charge carrier types: anions, cations or electrons. In some embodiments, the charge carriers are cations, e.g., hydrogen ions such as protons).

In some embodiments, the substrate of the enzyme can be redox active and the enzyme is directly responsible for generation of a charge carrier that is detected by an electrode. For example, the enzymefacilitates generation of the charge carrier near the antifouling layer surface, the conducting antifouling layer conducts the charge carrier and this impacts the applied voltage and/or current resulting in detection of the target analyte.

In some embodiments, the enzyme is a redox catalyst and, in the presence of the substrate for said enzyme, the substrate for the reported enzyme is oxidized or reduced, thereby generating a charge carrier. Non-limiting examples of redox active molecules that can be oxidized or reduced and can be substrates to a redox catalyst include, 3,3′,5,5′-tetramethylbenzidine (TMB), o-phenylenediamine dihydrochloride (OPD), 2,2′-Azinobis [3-ethylbenzothiazoline-6-sulfonic acid] (ABTS), p-Nitrophenyl Phosphate (PNPP), 3,3′-diaminobenzidine (DAB), 4-chloro-1-naphthol (4-CN), 5-bromo-4-chloro-3-indolyl-phosphate (BCIP), nitro blue tetrazolium (NBT), methylene blue, hydroquinone, ferrocene derivatives, and any combination thereof.

In some embodiments of any one of the aspects, the binding of the target analyte to the target binding molecule can be detected using the methods described in U.S. patent Ser. No. 10/753,940, content of which is incorporated herein by reference in its entirety. For example, where the detectable label comprises an enzyme, the method comprises contacting the labeling probe, e.g., labeling probe bound to the target analyte with a reporter enzyme substrate, an electroactive mediator and, optionally, a precipitating agent. It is noted the reporter enzyme substrate, the electroactive mediator and, optionally, the precipitating agent can be contacted with the labeling probe simultaneously or serially. In some embodiments, the reporter enzyme substrate, the electroactive mediator and, optionally, the precipitating agent are contacted simultaneously with the labeling probe. Without wishing to be bound by a reaction the reporter enzyme substrate, the electroactive mediator and, optionally, the precipitating agent with the enzyme conjugated with the label probe forms an electroactive precipitate which is locally deposited near or at the surface of the electrode.

Exemplary electroactive mediators include, but are not limited to, 3,3′,5,5′-tetramethylbenzidine (TMB), o-phenylenediamine dihydrochloride (OPD), 2,2′-Azinobis [3-ethylbenzothiazoline-6-sulfonic acid] (ABTS), p-Nitrophenyl Phosphate (PNPP), 3,3′-diaminobenzidine (DAB), 4-chloro-1-naphthol (4-CN), 5-bromo-4-chloro-3-indolyl-phosphate (BCIP), nitro blue tetrazolium (NBT), methylene blue, hydroquinone, ferrocene derivatives, and any combination thereof. In some embodiments, the electroactive mediator is TMB.

Exemplary precipitating agents include, but are not limited to, a water-soluble polymer, a pyrrolidinone polymer, a polyaniline, a polypyrrole, a polythiophene, alginic acid, methyl vinyl ether/maleic anhydride copolymer, dextran sulfate, carrageenan, and any combination thereof. In some embodiments, the precipitating agent is a pyrrolidinone polymer.

In some embodiments of any one of the aspects described herein, the reporter enzyme substrate, the electroactive mediator and, optionally, the precipitating agent are comprised in a composition for contacting with the labeling probe.

In some embodiments, the voltage applied corresponds to an electrochemical oxidation or reduction potential, or combination thereof, of the electroactive mediator in a fully or partially oxidized state. In some embodiments, the generated current corresponds to a reduction or oxidation current derived from reduction or oxidation of the fully or partially oxidized electroactive mediator. An exemplary voltage window includes, but is not limited to, about −0.2V as reduction potential to +0.2V as oxidation potential versus a reference electrode In some implementations, the capture agent is used at a concentration between about 10 and about 5000μ/mL. In some implementations, the capture agent is used at a concentration between about 50 and 1000μ/mL, such as between about 100 and 1000μ/mL, or between about 100 and about 1000μ/mL. In some implementations, the labeling probe is used at a concentration between about 0.1 and 100μ/mL, such as between about 0.5 and 50μ/mL, between about 1 and 20μ/mL, between about 1 and 8μ/mL, or between about 2 and 5μ/mL. In some implementations, the labeling probe includes streptavidin-polyHRP or a similar molecule for signal augmentation. In some implementations, the streptavidin-polyHRP concentration is between about 0.1 and about 100μ/mL, such as between about 0.5 and 50μ/mL, or between about 1 and 10μ/mL. The ranges of concentrations of capture agent and labeling probe can be used in any combination, such as 500μ/ml of capture agent in combination with 5μ/mL of labeling probe. The ranges of concentrations of capture agent, labeling probe and streptavidin-polyHRP also be used in any combination, such as 500μ/ml of capture agent, 5μ/ml of labeling probe and 2μ/mL streptavidin-polyHRP.

### Target Analytes

In some embodiments, the analyte is a biological analyte. In some embodiments, the analyte ion, molecule, oligomer, polymer, protein, peptide, polypeptide, peptidomimetic, nucleic acid, antigen, antibody, nucleic acid, toxin, biological threat agent such as spore, viral, cellular and protein toxin, carbohydrate, monosaccharide, disaccharide, oligosaccharide, polyol, and polysaccharide, lipid, peptidoglycan, cell, microbial matter, steroid, hormone, lipopolysaccharide, endotoxin, therapeutic agent, lipid-binding molecule, co-factor, small molecule, fatty acid, chemical, or combinations of these. The analyte is optionally an antigen or antibody indicative of infection or resistance to infection. The analyte is optionally a clinical chemistry analyte.

In some embodiments, the analyte is immunological or serological, for example an antigen or antibody.

In some embodiments the analyte is a hormone, for example a gynaecological hormone such as luteinizing hormone (LH), progesterone, estradiol or follicle-stimulating hormone. In preferred embodiments the probe detects LH. In some embodiments the probe is a LH specific antibody. In some embodiments the probe is an LH monoclonal antibody. Additionally, or alternatively the hormone may be a pregnancy hormone such as human chorionic gonatropin (hCG).

In some embodiments the analyte is a clinical chemistry analyte such as an ion, salt, mineral, metabolite, therapeutic drug, toxicology marker, drug of abuse, transport protein, enzyme, specific protein, lipoprotein or marker, for example diabetes or myocardial infarction markers. In some embodiments the analyte is a metabolite selected from the group of glucose, cholesterol, urea, lactic acid, bilirubin, creatinine, triglycerides. In preferred embodiments the probe is selected to detect glucose or cholesterol.

In some embodiments, the analyte is a tumour marker. Tumour markers can be used in guiding treatment decisions, monitoring treatment, predicting the change of recovery and to predict or monitor for tumour recurrence.

### Sample

In accordance with various embodiments described herein, a sample, including any fluid or specimen (processed or unprocessed) that is intended to be evaluated for the presence of an analyte can be subjected to methods, compositions, kits and systems described herein. The sample or fluid can be liquid, supercritical fluid, solutions, suspensions, gases, gels, slurries, and combinations thereof. The sample or fluid can be aqueous or non-aqueous.

In some embodiments, the sample can be an aqueous fluid. An aqueous fluid includes biological fluids as described below. Optionally, if the sample is water-based but not fluid, an aqueous solution can be added to produce a fluid sample.

In some embodiments, the sample can include a biological fluid obtained from a subject. Exemplary biological fluids obtained from a subject can include, but are not limited to, blood (including whole blood, plasma, cord blood and serum), lactation products (e.g., milk), amniotic fluids, sputum, saliva, urine, semen, cerebrospinal fluid, bronchial aspirate, perspiration, mucus, liquefied stool sample, synovial fluid, lymphatic fluid, tears, tracheal aspirate, and any combination thereof. In some embodiments, a biological fluid can include a homogenate of a tissue specimen (e.g., biopsy) from a subject. In one embodiment, a test sample can comprise a suspension obtained from homogenization of a solid sample or a fragment thereof obtained from a subject.

In some embodiments, the sample can include a fluid or specimen obtained from an environmental source. For example, the fluid or specimen obtained from the environmental source can be obtained or derived from food products or industrial food products, food produce, poultry, meat, fish, beverages, dairy products, water (including wastewater), surfaces, ponds, rivers, reservoirs, swimming pools, soils, food processing and/or packaging plants, agricultural places, hydrocultures (including hydroponic food farms), pharmaceutical manufacturing plants, animal colony facilities, and any combinations thereof.

In some embodiments, the sample can be a non-biological fluid. As used herein, the term “non-biological fluid” refers to any fluid that is not a biological fluid as the term is defined herein. Exemplary non-biological fluids include, but are not limited to, water, salt water, brine, drinking water, industrial water, brown water, sewerage, and mixtures thereof. Preferred non-biological fluids are drinking or industrial water or sewerage.

Optionally, the sample is pre-processed prior to contacting with the electrode or the organic field transistor.

### Sensors

In another aspect provided herein is a sensor comprising an electrode as described herein or an organic field effect transistor as described herein.

In the context of this specification, the term “sensor” refers a device that senses the presence and/or amount of something. For example, the sensor could sense the presence of a chemical such as glucose, a protein such as an antigen, or an antibody in a biological fluid.

A sensor has two basic components: the sensing surface (or receptor) and the transducer. The sensing surface interacts with the target analyte and the transducer converts this interaction into a readable electronic signal. The sensor performance characteristics depend on both the components. The sensor selectivity and affinity towards the target analyte depends solely on the sensing surface because the analyte interacts only at the sensing surface. Other performance metrics such as sensitivity, resolution, and calibration depend on both components.

The transducer is optionally a transistor such as an organic field effect transistor.

In some embodiments the sensor may be an eRapid chip. Such devices are generally described in “Enabling Multiplexed Electrochemical Detection of Biomarkers with High Sensitivity in Complex Biological Samples, Sanjay S. Timilsina, Pawan Jolly, Nolan Durr, Mohamed Yafia, and Donald E. Ingber, Acc. Chem. Res. 2021, 54, 18, 3529-3539”, which is hereby incorporated herein in its entirety.

The sensor is optionally an organic field effect transistor. The sensor may have a channel length, between the source and drain of electrodes, of between about 5 μm and about 50 μm, or between about 10 μm and about 30 μm, or about 20 μm, and a channel width of between about 1 mm and about 20 mm, or between about 1 mm and about 10 mm, or about 3 mm.

In some embodiments, the sensor has one or more fluid-contact surfaces, and the electrode or the organic field transistor is immobilized on at least a portion of the fluid contact surface. In some embodiments, the sensor has one or more wells. In some embodiments, each well of the sensor comprises an inner bottom surface on which one or more analyte specific electrodes or organic field transistors is immobilized. In some embodiments, the wells are open cells comprising open tops, enclosed sides and bottom, and one or more analyte-specific electrodes or organic field transistors immobilized on the inner fluid-contact surface of the wells. In some embodiments, the sensor comprises 1, 2, 3, 4, 5, 6, 8, 10, 12, 16, 24, 32, 48, 64, 96 or more open wells. In one embodiment the sensor is in the form of a 96-well microtiter plate.

In some embodiments, the wells are microfluidic flow cells comprising an enclosed top, sides and bottom, wherein the top of each flow cell includes a fluid inlet and a fluid outlet, and comprising one or more analyte-specific electrodes or organic field transistors immobilized on the inner fluid-contact surface of the wells. In some embodiments, the electrochemical sensor comprises 1, 2, 3, 4, 5, 6, 8, 10, 12, 16, 24, 32, 48, 64, 96 or more microfluidic flow cells. Another embodiment is in the form of a 96-well microtiter plate, wherein each well comprises an enclosed top having a fluid inlet and a fluid outlet. In some embodiments, the sensor comprises both one or more open cells and one or more flow cells. Each well contains an array of analyte-specific electrodes (e.g., 32 gold electrodes) or organic field transistor that can be individually modified with capture probes to bind the corresponding target analyte (e.g., pathogen, protein, carbohydrate, toxin, drug, etc.) present in the collected sample. In some embodiments, one sample is introduced into each well. In embodiments having two or more wells, portions of the same sample can be introduced into more than one well, or different samples can be introduced into different wells. Thus, in embodiments having multiple wells, multiple samples can be simultaneously assayed.

Also provided herein is the use of a sensor. In some embodiments, the sensor may be for sensing an analyte in a sample. The analyte is optionally a biological analyte. In an embodiment the analyte is an antibody, antigen, protein, peptide or chemical. The sample may be any aqueous solution but is preferably a biological fluid, more preferably a bodily fluid, and still more preferably, saliva.

Kits In another aspect, the present disclosure provides a kit comprising a composition, surface, electrode, organic field effect transistor or sensor described herein.

In addition to the above mentioned components, any embodiments of the kits described herein can include informational material. The informational material can be descriptive, instructional, marketing or other material that relates to the methods described herein and/or the use of the aggregates for the methods described herein. For example, the informational material can describe methods for using the kits provided herein to perform an assay for capture and/or detection of a target analyte. The kit can also include an empty container and/or a delivery device, e.g., which can be used to deliver a test sample to a test container.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the formulation and/or its use in the methods described herein. Of course, the informational material can also be provided in any combination of formats.

In some embodiments, the kit can contain separate containers, dividers or compartments for each component and informational material. For example, each different component can be contained in a bottle, vial, or syringe, and the informational material can be contained in a plastic sleeve or packet. In other embodiments, the separate elements of the kit are contained within a single, undivided container. For example, a collection of magnetic nanoparticles is contained in a bottle, vial or syringe that has attached thereto the informational material in the form of a label.

Some embodiments of the present invention can be defined as any of the following numbered embodiments:

Embodiment 1: A composition comprising a mixture of a proteinaceous material, a conductive element, and an anti-microbial agent.

Embodiment 2: The composition of embodiment 1, wherein the anti-microbial agent is covalently linked to the proteinaceous material or the conductive element.

Embodiment 3: The composition of embodiment 1 or 2, wherein the anti-microbial agent is covalently linked to the proteinaceous material or the conductive element via a linker.

Embodiment 4: The composition of embodiment 3, wherein the linker comprises genipin, polyethylene glycol (PEG), or glutaraldehyde.

Embodiment 5: The composition of embodiment 3 or 4, wherein a ratio of the anti-microbial agent to the cross-linker is from about 100:1 to about 10:1 (w/w).

Embodiment 6: The composition of any one of embodiments 1-5, wherein a ratio of the anti-microbial agent to the proteinaceous material is from about 30:1 to about 10:1 (w/w).

Embodiment 7: The composition of any one of embodiments 1-6, wherein a ratio of the anti-microbial agent to the conductive element is from about 20:1 to about 5:1 (w/w).

Embodiment 8: The composition of any one of embodiments 1-7, wherein a ratio of the proteinaceous material to the conductive element is from about 10:1 to about 1:1 (w/w).

Embodiment 9: The composition of any one of embodiments 1-8, wherein the anti-microbial agent is an anti-bacterial agent, anti-fungal agent or anti-viral agent.

Embodiment 10: The composition of any one of embodiments 1-9, wherein the anti-microbial agent is an anti-bacterial agent.

Embodiment 11: The composition of embodiment 10, wherein the anti-bacterial agent is selected from the group consisting of macrolides or ketolides such as erythromycin, azithromycin, clarithromycin, and telithromycin; beta-lactams including penicillin, cephalosporin, and carbapenems such as carbapenem, imipenem, and meropenem; monolactams such as penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, cefiriaxone, cefoperazone, ceftazidime, cefixime, cefpodoxime, ceftibuten, cefdinir, cefpirome, cefepime, cefadroxil, ceftriaxone, ceftobiprole and astreonam; quinolones such as nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, ganefloxacin, gemifloxacin and pazufloxacin; antibacterial sulfonamides and antibacterial sulphanilamides, including para-aminobenzoic acid, sulfadiazine, sulfisoxazole, sulfamethoxazole and sulfathalidine; aminoglycosides such as streptomycin, neomycin, kanamycin, paromycin, gentamicin, tobramycin, amikacin, netilmicin, spectinomycin, sisomicin, dibekalin and isepamicin; tetracyclines such as tetracycline, chlortetracycline, demeclocycline, minocycline, oxytetracycline, methacycline, doxycycline; rifamycins such as rifampicin (also called rifampin), rifapentine, rifabutin, bezoxazinorifamycin and rifaximin; lincosamides such as lincomycin and clindamycin; glycopeptides such as vancomycin and teicoplanin; streptogramins such as quinupristin and daflopristin; oxazolidinones such as linezolid; polymyxin, colistin and colymycin; trimethoprim, bacitracin, and phosphonomycin.

Embodiment 12: The composition of any one of embodiments 1-9, wherein the anti-microbial agent is an anti-fungal agent.

Embodiment 13: The composition of embodiment 12, wherein the anti-fungal agent is selected from the group consisting of azoles (e.g., barleyconazole, butoconazole, clortrimazole, econazole, fluconazole, isavuconazole, itraconazole, ketoconazole, miconazole, oxyconazole, posaconazole, ravuconazole, saperconazole, sulconazole, tercocnazole, tioconazole, voriconazole, and ciclopirox), polyenes (e.g., natamycin, lucensomycin, nystatin, amphotericin B, etc.), echinocandins (e.g., Cancidas), pradimicins (e.g., beanomicins, nikkomycins, sordarins, allylamines, etc.), Triclosan, Piroctone, fenpropimorph, terbinafine, cyclopyroxolamine, flucitocin, griseofulvin haloprozin, tolnaftate, naphthypine, hydrochloride, morpholine, butenapin, undecylenic acid, propionic acid, and derivatives and analogs thereof.

Embodiment 14: The composition of any one of embodiments 1-13, wherein the anti-microbial agent comprises a functional group for cross-linking, e.g., an amino (NH₂) group.

Embodiment 15: The composition of any one of embodiments 1-9, wherein the anti-microbial agent is an antimicrobial peptide or polymer.

Embodiment 16: The composition of any one of embodiments 1-9, wherein the anti-microbial agent is a metal particle.

Embodiment 17: The composition of embodiment 16, wherein anti-microbial agent is titanium oxide, copper, or silver nanoparticles.

Embodiment 18: The composition of any one of embodiments 1-17, wherein the composition comprises at least two (e.g., 3, 4, 5, or more) different anti-microbial agents.

Embodiment 19: The composition of any one of embodiments 1-18, wherein the proteinaceous material is denatured.

Embodiment 20: The composition of any one of embodiments 1-19, wherein the proteinaceous material is non-reversibly denatured.

Embodiment 21: The composition of any one of embodiments 1-20, wherein the proteinaceous material is a globular protein.

Embodiment 22: The composition of any one of embodiments 1-21, wherein the proteinaceous material is a non-glycosylated protein.

Embodiment 23: The composition of any one of embodiments 1-22, wherein the proteinaceous material is a serum albumin protein.

Embodiment 24: The composition of any one of embodiments 1-23, wherein the proteinaceous material is bovine serum albumin (BSA) or human serum albumin (HSA).

Embodiment 25: The composition of any one of embodiments 1-24, wherein the proteinaceous material is cross-linked with the conductive element.

Embodiment 26: The composition of any one of embodiments 1-25, wherein the proteinaceous material is cross-linked to the conductive element by a linker.

Embodiment 27: The composition of embodiment 26, wherein the linker is genipin, polyethylene glycol, or glutaraldehyde.

Embodiment 28: The composition of any one of embodiments 1-27, wherein the proteinaceous material is cross-linked to itself.

Embodiment 29: The composition of any one of embodiments 1-28, wherein the proteinaceous material is cross-linked to itself by a linker.

Embodiment 30: The composition of embodiment 29 wherein the linker is genipin, polyethylene glycol, or glutaraldehyde.

Embodiment 31: The composition of any one of embodiments 1-30, wherein the conducting material comprises conductive particles, conductive rods, conductive fibers, conductive nano-particles, conductive polymers, conductive nano-flakes, conductive nanotubes, semi-conductive particles, semi-conductive rods, semi-conductive fibers, semi-conductive nano-particles, semi-conductive nano-flakes, semi-conductive nanotubes, or semi-conductive polymers.

Embodiment 32: The composition of any one of embodiments 1-31, wherein the conducting material is a metal, a metalloid, a conducting polymer, a conducting carbon based material, or any combination thereof.

Embodiment 33: The composition of any one of embodiments 1-32, wherein the conducting material comprises an allotrope of carbon atoms arranged in a hexagonal lattice.

Embodiment 34: The composition of any one of embodiments 1-33, wherein the conducting material is graphite, functionalised graphene oxide, reduced graphene oxide, or carbon nano-tubes (CNTs).

Embodiment 35: The composition of embodiment 34, wherein the carbon nanotubes are carboxylated carbon nanotubes or aminated carbon nanotubes.

Embodiment 36: The composition of embodiment 34, wherein the reduced graphene oxide is a carboxylated reduced graphene oxide or an aminated reduced graphene oxide.

Embodiment 37: The composition of any one of embodiments 1-32, wherein the conductive material comprises gold.

Embodiment 38: The composition of any one of embodiments 1-37, wherein the mixture further comprises a target binding molecule capable of binding with a target molecule.

Embodiment 39: The composition of embodiment 38, wherein the target binding molecule is covalently linked to the proteinaceous material, or the conductive or semi-conductive material.

Embodiment 40: The composition of embodiment 38 or 39, wherein the target binding molecule is a receptor, a ligand for a receptor, an antibody, antigen binding fragment of an antibody, an antigen, an enzyme or a nucleic acid.

Embodiment 41: The composition of any one of embodiments 38-40, wherein the target binding molecule is an antibody, an antigen binding fragment of an antibody or an antigen.

Embodiment 42: The composition of any one of embodiments 1-41, wherein the composition further comprises a polymer, e.g., a polymer selected from the group consisting of polyethylene glycol (PEG), alginate, polytetrafluoroethylene (PTFE), polyacetylene (PA), polyaniline (PANI), polypyrrole (PPy), polythiophene (PTH), poly(para-phenylene) (PPP), poly(phenylenevinylene) (PPV), and polyfuran (PF).

Embodiment 43: The composition of any one of embodiments 1-41, wherein the composition further comprises a a therapeutic agent, e.g., anti-inflammatory drugs sirolimus, everolimus, biolimus (A9), zotarolimus (ABT-578), tacrolimus, and pimecrolimus, genistein, steroids (dexamethasone, prednisolone, methylprednisolone and hydrocortisone), fluocinolone acetonide, hormones etc.

Embodiment 44: The composition of any one of embodiments 1-42, wherein the mixture is a homogenous mixture.

Embodiment 45: The composition of any one of embodiments 1-43, wherein the composition is antifouling.

Embodiment 46: The composition of any one of embodiments 1-44, wherein the composition has antimicrobial activity.

Embodiment 47: A surface with antifouling properties, wherein the surface comprises an antifouling coating layer comprising a composition of any one of embodiments 1-46.

Embodiment 48: The surface of embodiment 47, wherein the antifouling coating layer is directly or indirectly connected with an electrode.

Embodiment 49: An electrode comprising: (i) a conductive substrate (e.g., an electrically conductive substrate); and (2) an antifouling coating layer on at least a portion of a surface of the conductive substrate, and wherein the antifouling coating layer comprises a composition of any one of embodiments 1-46.

Embodiment 50: The electrode of embodiment 49, wherein the conductive substrate comprises gold, silver, copper, platinum, aluminum, stainless steel, tungsten, indium tin oxide, titanium, lead, nickel, palladium, zirconium, niobium, tantalum, chromium, molybdenum, manganese, rhenium, ruthenium, rhodium, iridium, silicon, polyimide, parylene, benzocyclobutene, carbon, graphite, or any combination thereof.

Embodiment 51: The electrode of embodiment 49 or 50, wherein the conductive substrate comprises a flexible substrate.

Embodiment 52: The electrode of embodiment 51, wherein the flexible substrate comprises polyethylene terephthalate, polyethylene naphathalate, polyimides, polymeric hydrocarbons, celluloses, plastics, polycarbonates, polystyrenes, or any combination thereof.

Embodiment 53: The electrode of any one of embodiments 49-52, wherein the antifouling coating layer is adapted for contact with an analyte or a sample comprising an analyte.

Embodiment 54: The electrode of any one of embodiments 49-53, wherein the electrode is a planar or 3-dimensional electrode.

Embodiment 55: The electrode of any one of embodiments 49-54, wherein the antifouling coating layer has a thickness of from about 0.1 nm to about 10 μm.

Embodiment 56: An organic field effect transistor comprising: (a) a source electrode; (b) a drain electrode; (c) an organic conducting or semiconducting layer connecting the source electrode to the drain electrode; and (d) an antifouling coating layer on at least a portion of the surface of the organic conducting or semiconducting layer, and wherein the antifouling coating layer comprises a composition of any one of embodiments 1-46.

Embodiment 57: The organic field effect transistor of embodiment 56, wherein the organic conducting or semiconducting layer has a thickness of from about 0.1 nm to about 10 μm.

Embodiment 58: The organic field effect transistor of embodiment 56 or 57, wherein the antifouling coating layer has a thickness of from about 0.1 nm to about 10 μm.

Embodiment 59: The organic field effect transistor of any one of embodiments 56-58, wherein the organic conducting or semiconducting layer comprises one or more organic compounds having conducting and/or semiconducting properties.

Embodiment 60: The organic field effect transistor of embodiment 59, wherein the organic compounds having conducting and/or semiconducting properties are selected from the group consisting of polyacetylenes, porphyrins, phthalocyanins, fullerenes, polyparaphenylenes, polyphenylenevinylenes, polyfluorenes, polythiophenes, polypyrroles, polypyridines, polycarbazoles, polypyridinevinylenes, polyarylvinylenes, poly (p-phenylmethylvinylenes), derivatives and co-polymers thereof, and any combination thereof.

Embodiment 61: The organic field effect transistor of embodiment 59 or 60, wherein the organic compounds having conducting and/or semiconducting properties are selected from the group consisting of poly(3-hexylthiophene) (P3HT), perfluoro-3,6-dioxa-4-methyl-7-octenesulfonic acid copolymer, tetrafluoroethylene-perfluoro-3,6-dioxa-4-methyl-7-octenesulfonic acid copolymer (e.g. nafion), poly(9,9-dioctylfluorene-2,7-diyl-co-bis-N, N-(4-butylphenyl)-bis-N, N-phenyl-1,4-phenylenediamine), poly(9,9-dioctylfluorene-2,7-diyl-co-benzothiadiazole), (6,6)-phenyl-C61-butyric acid methyl ester, poly(2-methoxy-5-(2′-ethyl-hexyloxy)-1,4-phenylene vinylene), poly(4-vinylphenol) (PVP), and copolymers thereof, and any combination thereof.

Embodiment 62: The organic field effect transistor of any one of embodiments 56-61, wherein the organic field effect transistor further comprises a conducting polymer layer between the organic conducting or semiconducting layer and the antifouling layer.

Embodiment 63: The organic field effect transistor of embodiment 62, wherein the conducting polymer layer comprises one or more of fluorinated polymer, poly methyl methacrylate (PMMA), polyhydroxyethylmethacrylate (PolyHEMA), polyacetylenes, porphyrins, phthalocyanins, fullerenes, polyparaphenylenes, polyphenylenevinylenes, polyfluorenes, polythiophenes, polypyrroles, polypyridines, polycarbazoles, polypyridinevinylenes, polyarylvinylenes, poly(p-phenylmethylvinylenes), and derivatives, co-polymers, mixtures and blends thereof.

Embodiment 64: The organic field effect transistor of embodiment 62 or 63, wherein the conducting polymer layer comprises one or more of poly(3-hexylthiophene) (P3HT), perfluoro-3,6-dioxa-4-methyl-7-octenesulfonic acid copolymer, tetrafluoroethylene-perfluoro-3,6-dioxa-4-methyl-7-octenesulfonic acid copolymer (e.g. nafion), poly(9,9-dioctylfluorene-2,7-diyl-co-bis-N, N-(4-butylphenyl)-bis-N, N-phenyl-1,4-phenylenediamine), poly(9,9-dioctylfluorene-2,7-diyl-co-benzothiadiazole), (6,6)-phenyl-C61-butyric acid methyl ester, poly(2-methoxy-5-(2′-ethyl-hexyloxy)-1,4-phenylene vinylene), poly(4-vinylphenol) (PVP), and copolymers thereof, and any combination thereof.

Embodiment 65: The organic field effect transistor of any one of embodiments 62-64, wherein the conducting polymer layer has a thickness of from about 0.1 nm to about 10 μm.

Embodiment 66: The organic field effect transistor of any one of embodiments 56-65, wherein the organic field effect transistor further comprises gate electrode in contact with the antifouling coating layer or the organic conducting or semiconducting layer.

Embodiment 67: The organic field effect transistor of any one of embodiments 56-66, wherein the antifouling coating layer and/or the organic conducting or semiconducting layer is configured to be connected to an ohmic conductor for applying a gate voltage to said organic conducting or semiconducting layer (e.g., via antifouling layer).

Embodiment 68: The organic field effect transistor of embodiment 67, wherein the antifouling coating layer is configured to be connected to the ohmic conductor.

Embodiment 69: The organic field effect transistor of embodiment 67 or 68, wherein the organic field effect transistor further comprises an ohmic conductor in contact with the antifouling coating layer and/or the organic conducting or semiconducting layer.

Embodiment 70: The organic field effect transistor of any one of embodiments 67-69, wherein the ohmic conductor is a gate electrode.

Embodiment 71: A method for detecting a target analyte in a sample, the method comprising: contacting a sample suspected of comprising a target analyte with an electrode of any one the preceding embodiments and detecting binding of the target analyte with the target binding ligand.

Embodiment 72: The method of embodiment 71, wherein said detecting the binding of the target molecule with the target binding ligand comprises applying a voltage to the electrode.

Embodiment 73: The method of embodiment 71 or 72, wherein said detecting the binding of the target molecule with the target binding ligand comprises measuring a current generated from electrode.

Embodiment 74: A method for detecting a target analyte in a sample, the method comprising: contacting a sample suspected of comprising a target analyte with an organic field effect transistor of any one of the preceding embodiments and detecting binding of the target analyte with the target binding ligand.

Embodiment 75: The method of embodiment 74, wherein said detecting the binding of the target molecule with the target binding ligand comprises applying a voltage to the drain electrode.

Embodiment 76: The method of embodiment 74 or 75, wherein said detecting the binding of the target molecule with the target binding ligand comprises applying a voltage to the ohmic conductor.

Embodiment 77: The method of any one of embodiments 74-76, wherein said detecting the binding of the target molecule with the target binding ligand comprises grounding the source electrode.

Embodiment 78: The method of any one of embodiments 74-77, wherein said detecting the binding of the target molecule with the target binding ligand comprises detecting a drain current.

Embodiment 79: The method of any one of embodiments 71-78, wherein said detecting the binding of the target molecule with the target binding molecule comprises contacting a second target binding molecule to the target molecule, wherein the second target binding molecule comprises a detectable label.

Embodiment 80: The method of embodiment 79, wherein said contacting with the second target binding molecule is prior to contacting the sample with the electrode or the organic field effect transistor.

Embodiment 81: The method of embodiment 79, wherein said contacting with the second target binding molecule is after contacting the sample with the electrode or the organic field effect transistor.

Embodiment 82: The method of any one of embodiments 79-81, wherein the detectable label comprises an enzyme, a fluorophore, a chemiluminescent label, colloidal gold, colored glass or plastic (e.g., polystyrene, polypropylene, and latex) beads, a radiolabel, a quantum dot, or any combination thereof.

Embodiment 83: The method of any one of embodiments 79-84, wherein the detectable label comprises an enzyme.

Embodiment 84: The method of embodiment 85, wherein the enzyme is a peroxidase, alkaline phosphatase, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase or acetylcholinesterase.

Embodiment 85: The method of embodiment 83 or 84, wherein the enzyme is a peroxidase or alkaline phosphatase.

Embodiment 86: The method of any one of embodiments 83-85, wherein the method further comprises contacting the enzyme with a substrate of the enzyme.

Embodiment 87: The method of any one of embodiments 79-86, wherein the detectable label facilitates generation of a charge carrier.

Embodiment 88: The method of embodiment 87, wherein said detecting the binding of the target molecule with the target binding molecule comprises detecting the charge carrier.

Embodiment 89: The method of any one of embodiments 79-88, wherein the target analyte is a peptide, a polypeptide, a peptidomimetic, a nucleic acid, an oligosaccharide, a polysaccharide, an amino acid, nucleoside, a nucleotide, a carbohydrate, a lipid, a peptidoglycan, a cell, microbial matter, an antigen, a lipid, a steroid, a hormone, a lipopolysaccharide, an endotoxin, a therapeutic agent, a lipid-binding molecule, a cofactor, a small molecule, a toxin, a biological threat agent (e.g., spore, viral, cellular and protein toxin), or any combination thereof.

Embodiment 90: The method of any one of embodiments 79-89, wherein the target analyte is a protein, an antibody, an antigen binding fragment of an antibody, an antigen, a hormone, or a metabolite.

Embodiment 91: The method of any one of embodiments 79-90, wherein the target analyte is a tumour marker or a clinical chemistry target.

Embodiment 92: The method of any one of embodiments 79-91, wherein the sample is a biological sample (e.g., blood, saliva, amniotic fluid, sputum, urine, semen, cerebrospinal fluid, bronchial aspirate, perspiration, mucus, liquefied stool sample, synovial fluid, lymphatic fluid, tears, tracheal aspirate, lactation product, and any combination thereof).

Embodiment 93: The method of any one of embodiments 79-92, wherein the sample is a food, an ingredient for preparing a food, poultry, meat, fish, beverage, or dairy product.

Embodiment 94: The method of any one of embodiments 79-93, wherein the sample is a non-biological sample (e.g., water, salt water, pond water, river water, reservoir water, brine, drinking water, industrial water, brown water, waste water, sewerage, soil, and mixtures thereof.

Embodiment 95: The method of any one of embodiments 79-94, wherein the sample is pre-processed prior to contacting with the electrode or the organic field effect transistor.

Embodiment 96: A sensor comprising an electrode of any one of embodiments 49-55 or an organic field effect transistor of any one embodiments 56-70.

Embodiment 97: The sensor of embodiment 96, wherein the sensor comprises a fluid-contact surface and the electrode or the organic field effect transistor is immobilized on at least a portion of the fluid-contact surface.

Embodiment 98: The sensor of embodiment 96 or 97, wherein the sensor comprises one or more microfluidic flow cells.

Embodiment 99: The sensor of any one of embodiments 96-98, wherein the sensor comprises one or more microfluidic flow cells.

Embodiment 100: The sensor of any one of embodiments 97-99, wherein the fluid-contact surface further comprises a positive control electrode and/or a negative control electrode immobilized thereon.

Embodiment 101: Use of an electrode of any one of embodiments 49-55 or an organic field effect transistor of any one embodiments 56-70 for detecting a target analyte in a sample.

Embodiment 102: A kit comprising a composition, surface, electrode, organic field effect transistor or sensor of any one of the preceding embodiments.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As used herein the term “comprising” or “comprises” is used in reference to compositions, methods, and respective component(s) thereof, that are essential to the invention, yet open to the inclusion of unspecified elements, whether essential or not. In other words, except where the context requires otherwise, the term “comprise” and variations of the term, such as “comprising”, “comprises” and “comprised”, are not intended to exclude further additives, components, integers or steps.

The singular terms “a,” “an,” and “the” include plural referents unless context clearly indicates otherwise. Similarly, the word “or” is intended to include “and” unless the context clearly indicates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “binding” or “bound” generally refers to a reversible binding of one molecule to molecule via, e.g., van der Waals force, hydrophobic force, hydrogen bonding, and/or electrostatic force. The binding interaction between two molecules can be described by a dissociation constant (K_{d}) or association constant (K).

Specific elements of any of the disclosed embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example 1: RepelSense: A Biocompatible Sensor Coating for In Vivo Monitoring That Actively Inhibits Microbial Proliferation and Fibrotic Attachment

Biofouling and foreign body response have deleterious effects on sensor functionality and longevity, seriously impeding the implementation of implantable/wearable biosensors for long-term monitoring. Here is described a novel nanocomposite coating consisting of covalently interlinked bovine serum albumin (BSA), amine-terminated reduced graphene, and antibiotics via a crosslinker, that actively combats biofouling while retaining high electroconductivity and excellent electrochemical immunosensor behavior. The coating's antimicrobial efficacy is investigated via a micro broth dilution assay and its biocompatibility is demonstrated with human-isolated monocytes and fibroblasts via flow cytometry, microscopy, MSD analysis, and an alamarBlue cell viability assay. Results demonstrate the capability of this coating to mitigate microbial proliferation and fibrotic adhesion. At the same time, its electrochemical stability can be maintained after 21 days of exposure to BSA, a potential blocking agent, and the coating's applicability is showcased with an immunosensor that detects cytokines in culture medium and human plasma.

### Introduction

Implementation of implantable/wearable biosensors is greatly hindered by biofouling, as non-specific absorption of cells and proteins passivate and deteriorate the electrode, compromising electrochemical performance. [1,2] Moreover, implantation of a foreign body elicits unfavorable tissue reactions known as the foreign body response (FBR), isolating the sensor from analytes of interest, compromising device sensitivity/functionality, and fostering subsequent biofilm formation, all of which contribute to the elevation of infection risk and implant failure. [3-7] Thus, mitigating biofouling, improving biocompatibility, and minimizing both acute and chronic states of FBR are crucial to securing the clinical translation of long-term, continuous biosensors.

The inventors previously reported, antifouling coating, a 3D nanocomposite of bovine serum albumin (BSA) integrated with electroconductive pentaamine functionalized reduced graphene oxide flakes (prGOx) which is further stabilized by crosslinking with glutaraldehyde (GTA). [8,9] This unique structure facilitates electron transfer while effectively preventing protein diffusion, serving as a protective shield for the electrode. [8,10] However, there are potential cytotoxicity issues associated with GTA due to the presence of unreacted moieties and toxic by-products, which may limit its applicability in wearable or implantable devices. [11,12] Additionally, some microorganisms could bypass this antifouling barrier, leveraging sophisticated attachment factors like non-proteinaceous adhesins, including polysaccharides and extracellular DNA. [13,14]

To enhance coatings with active antimicrobial features, common strategies involve embedding or releasing bactericidal agents such as ions, nanoparticles, salts, antibiotics, etc. [15-17] While highly effective in the short-term, agent-releasing coatings encounter challenges regarding loading capacity, reservoir exhaustion, cellular toxicity, and potential induction of antimicrobial resistance, which restrict their long-term use. [18] Conversely, coatings employing contact-killing methods are effective without exerting adverse effects on surrounding tissue but they are prone to biofouling as dead bacteria accumulate on their surfaces. [18-20] For electrochemical sensors, these approaches are further challenged by inherent trade-offs between coating thickness and electrochemical and antimicrobial properties. While thin-film coatings (5-15 nm) offer optimal electrochemical attributes, antimicrobial efficacy is accentuated at higher thicknesses, such as in zinc oxide coatings (200-600 nm) which compromise sensor sensitivity. [21] Efficiently incorporating an antimicrobial agent into thinner coatings also poses challenges, as seen in past studies on gold nanoparticles in the nanocomposite, revealing issues with integration and BSA adsorption, attributed to electrostatic repulsion. [22]

In response to these challenges, presented herein is RepelSense, a proprietary and safe thin-film nanocomposite coating for wearable and/or implantable electrochemical sensors. This novel technology exhibits antimicrobial activity through active and passive strategies while retaining outstanding electrochemical behavior, as verified up to 21 days. Moreover, this greatly improved biocompatibility prevents unfavorable cell activation and attachment, which can contribute to localized discomfort, irritation, pain, erythema, and edema, as well as initiation of FBR. [5,23,24] An antibody-based sensor resulting from a sandwich ELISA involving two important inflammatory biomarkers, MIP-1b and IL-6, is characterized in culture medium and human plasma. In addition, this technology offers high cost-effectiveness and manufacturability due to a single-pot recipe of low-cost components and a highly scalable drop-casting method. This novel technology holds the potential to substantially increase the lifespan and functionality of implantable/wearable biosensors, presenting exciting avenues for continuous monitoring of a plethora of analytes.

### Results and Discussion

### Antimicrobial Efficacy and Stability Maintained Over Time

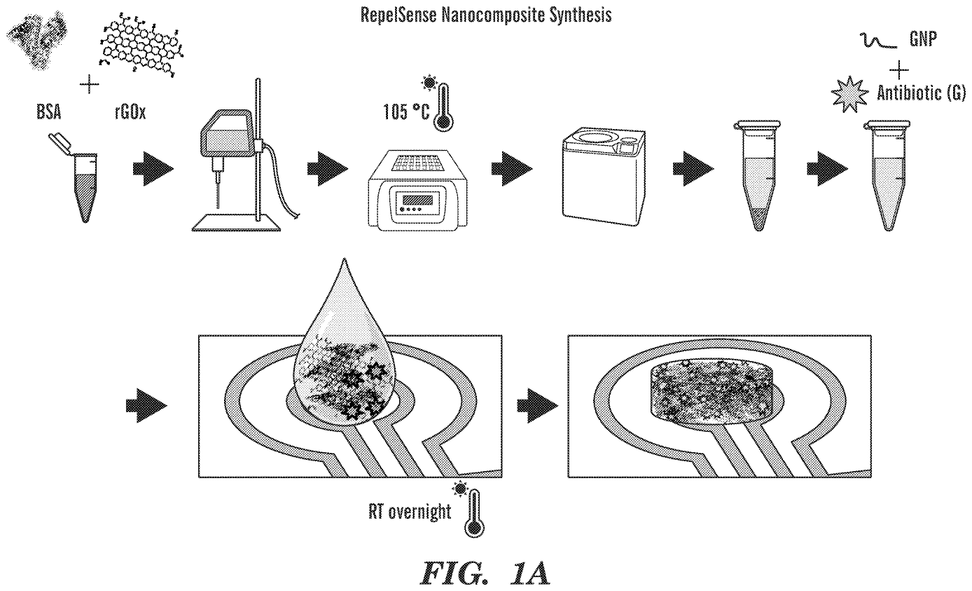

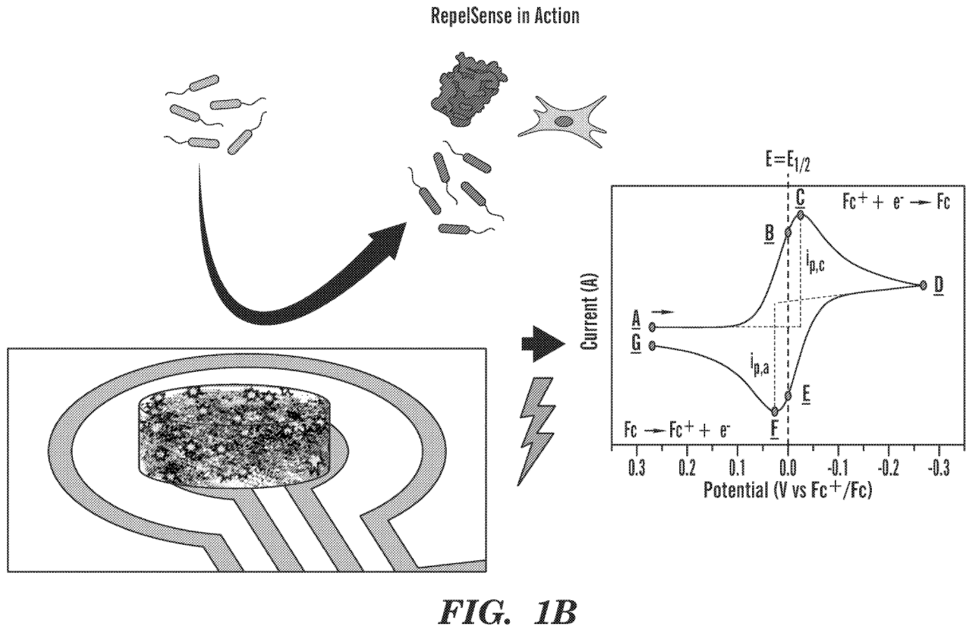

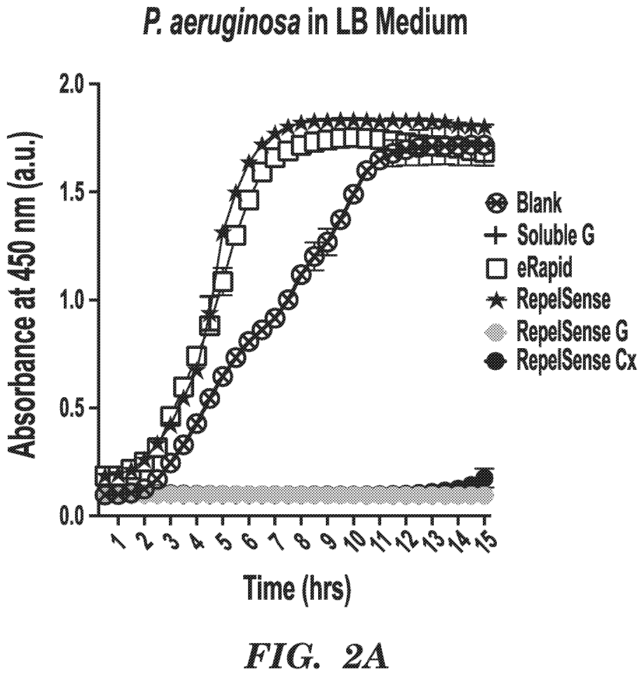

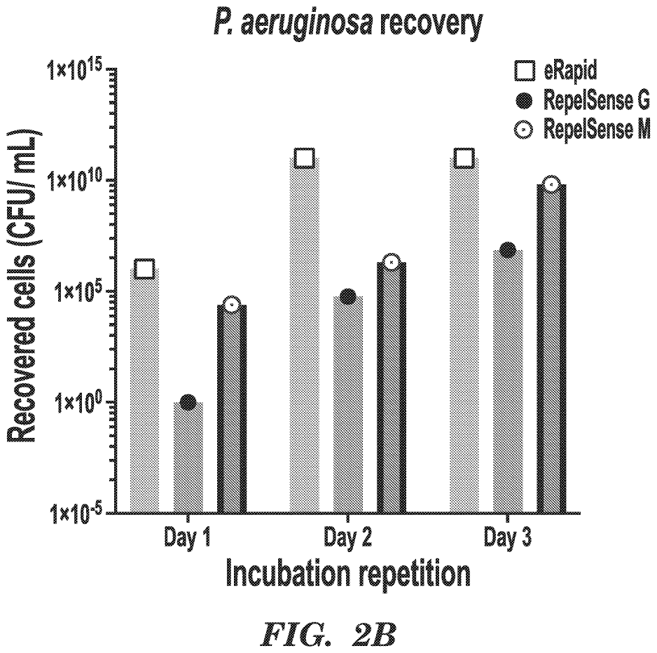

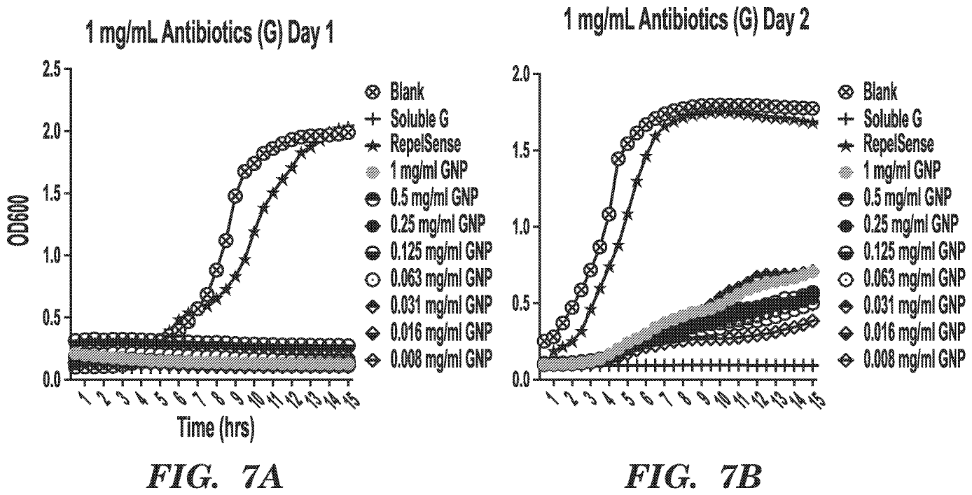

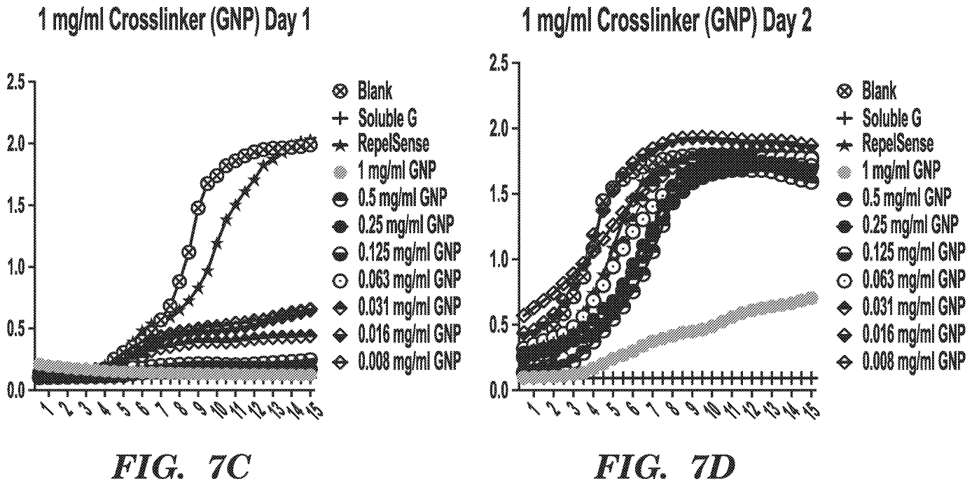

Biosensor surface modification approaches, such as antimicrobial coatings, could prevent initial bacterial adhesion and biofilm formation, enabling prolonged sensor application and wear time. To confer the nanocomposite coating (RepelSense) with enhanced antimicrobial protection, we cross-linked antibiotics containing primary amine groups (such as gentamicin (G) and ceftriaxone (Cx)) with prGOx/BSA/GNP resulting in an antimicrobial nanocomposite coating (RepelSense G & RepelSense Cx, respectively) (FIG. 1). G is an aminoglycoside broad-spectrum antibacterial that works against common implant-associated pathogens such as Staphylococcus spp. and aerobic Gram-negative organisms as well as established biofilms, while Cx is a third-generation cephalosporin used to treat a multitude of infections. [25,26] Both RepelSense G, and RepelSense Cx demonstrated excellent antimicrobial effectiveness against P. aeruginosa, a gram-negative bacterium, over 15 h, on par with a saturating dose of soluble antibiotic (soluble G, 100 μg mL−1 gentamicin), verified through a simple broth microdilution assay, compared with RepelSense lacking an antimicrobial component (i.e., meropenum (M)) and eRapid coatings (FIG. 2). To optimize antimicrobial activity, we assessed the ideal ratio of antibiotics to the cross-linker, GNP, using RepelSense G as a model, testing combinations of two-fold dilutions from 1 mg mL−1 to 0.008 mg mL−1 of both G and GNP. The combination of G and GNP each at a concentration of 1 mg mL−1 (1:1 ratio) was found to optimally retain antimicrobial properties (FIG. 7).

In prior studies, we observed enhanced antifouling retention in the nanocomposite due to covalent interaction between the conducting nanomaterial (prGOx) and the BSA matrix, facilitating dispensability and maintaining composite structure. [8,10] We hypothesized that the RepelSense coating sustained antimicrobial activity from covalent crosslinking between the s-amines of lysine residues in BSA and primary amine groups of the integrated antibiotic. To validate this, we assessed the antimicrobial efficacy of RepelSense when fortified with M, an antibiotic lacking primary amine or carboxyl functionalities (designated RepelSense M), and compared this to RepelSense G and eRapid coatings. Nanocomposite-coated chips were inoculated with ~102 CFU mL−1 of P. aeruginosa, incubated for 24 h at 37° C., washed, and counted, with the process repeated for 3 days. The amine-enriched RepelSense G demonstrated superior antimicrobial potency compared to both eRapid and RepelSense M coatings (FIG. 2B), validating our hypothesis. This covalent cross-linking attachment thus proved crucial for sustained efficacy and minimal antibiotic leaching and was considered a prerequisite for subsequent iterations.

### Improved Biocompatibility of Repelsense Evident in Cell Viability and Functionality

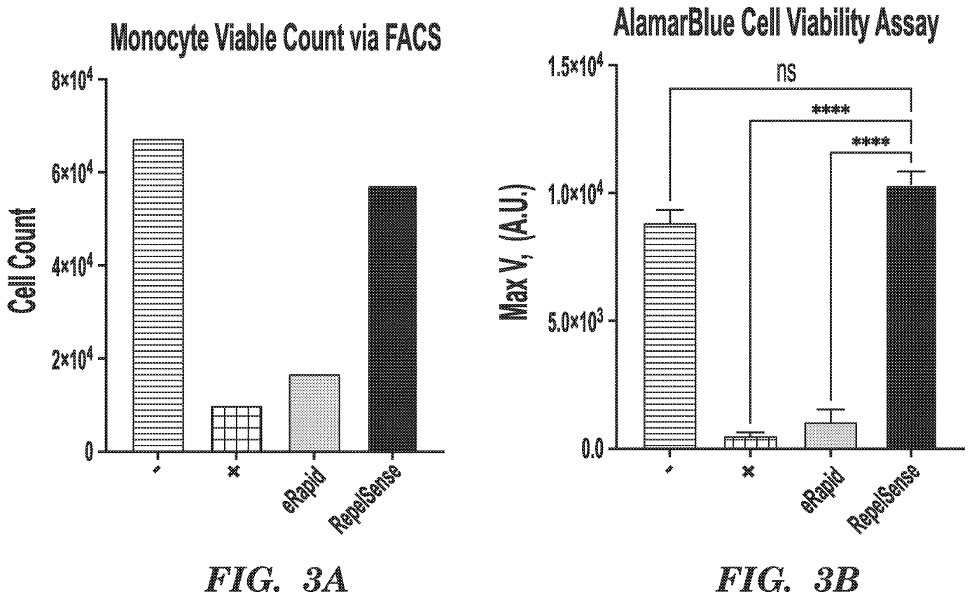

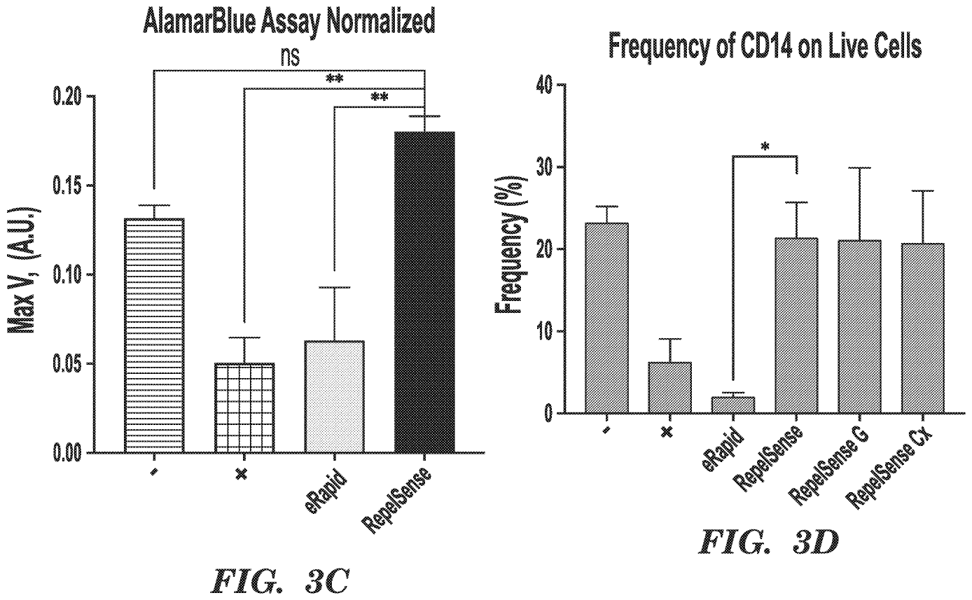

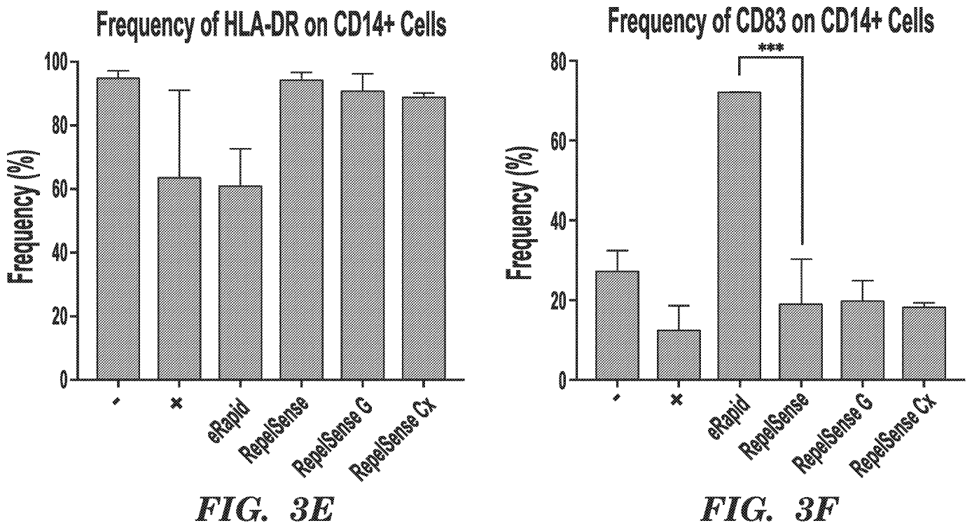

A nanocomposite's biocompatibility is pivotal for biosensor application and prolonged use. [23,24] RepelSense's biocompatibility was augmented by replacing the cytotoxic crosslinker, GTA, with non-toxic GNP, ensuring sensor functionality. GNP is a natural crosslinking agent with multiple advantages such as biocompatibility, non-cytotoxicity, and stability, positioning it as an eco-friendly and cost-effective alternative to traditional crosslinkers. [11,27] To assess RepelSense's enhanced biocompatibility, monocytes from peripheral blood mononuclear cells (PBMCs) of three human donors were cultured on nanocomposite-coated wells and incubated at 37° C. for set durations. Cell viability was determined using flow cytometry (FIG. 3A), and metabolic activity was measured via an alamarBlue assay with periodic fluorescence intensity recordings at 37° C. (FIG. 3B). Post-normalization, monocytes incubated with RepelSense showed heightened metabolic activity compared to the cytotoxic control (p<0.05) (FIG. 3C). Conversely, eRapid-coated samples displayed reduced cell viability, akin to the cytotoxic control (p<0.05), emphasizing that replacing GTA with GNP as a crosslinker in RepelSense significantly improves monocyte viability and metabolic function.

This improved biocompatibility was maintained despite the crosslinking of antibiotics, as verified through the expression of co-stimulatory molecules CD14, Human leukocyte antigen DR (HLA-DR), and CD83 on freshly isolated monocytes placed onto RepelSense coatings (RepelSense G, RepelSense Cx). CD14 pertains to the classical CD14++CD16− monocyte subpopulation, whereas CD14-CD16+ monocytes precede pro-fibrotic M2 macrophages, which are crucial players in infection, inflammation, and disease pathogenesis. [28-30] Similarly, while indicative of immune deficiency in critically ill patients, HLA-DR also correlates with reduced antigen presentation and proinflammatory cytokine release, providing a prediction of mortality or risk of secondary infection. [30,31] Overall CD14 expression and subsequent HLA-DR expression on CD14+cells were lower for the eRapid coating condition and the positive (cytotoxic) control. In contrast, RepelSense coating conditions showed similar expression of CD14, and HLA-DR expression on CD14+ cells, compared to the negative control (FIG. 3D & FIG. 3E) emphasizing enhanced cell functionality. Additionally, the eRapid coating condition showed elevated CD83, an antigen-presenting cell activation marker, suggesting heightened monocyte activation propensity compared to RepelSense coating conditions. [32,33]

### RepelSense Prevents Initial Fibrotic Attachment While Maintaining Cell Metabolic Function

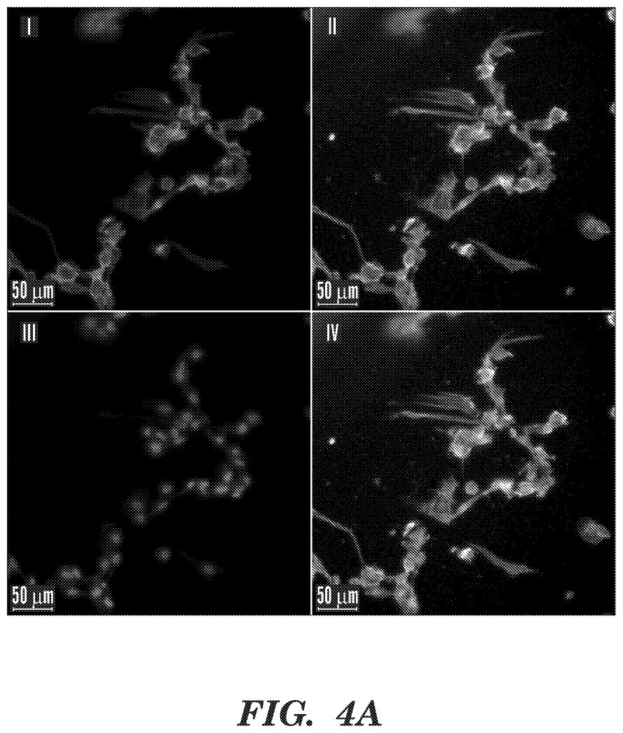

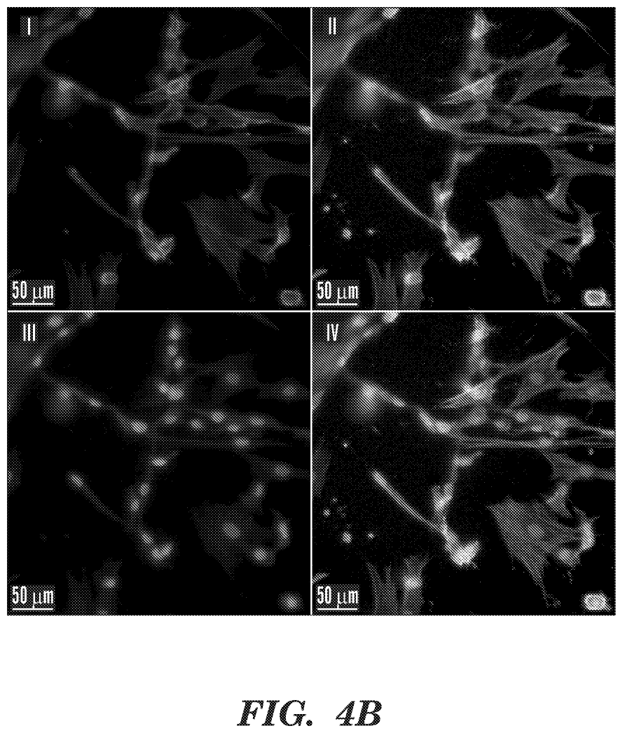

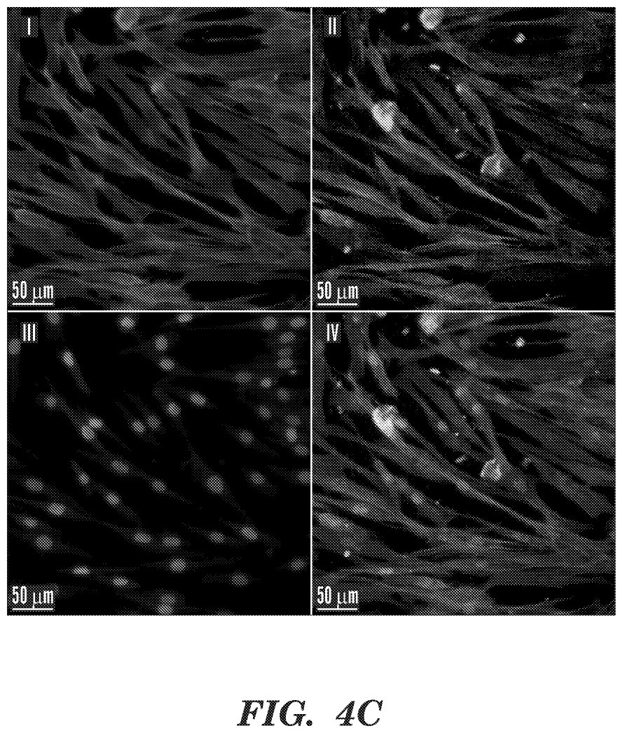

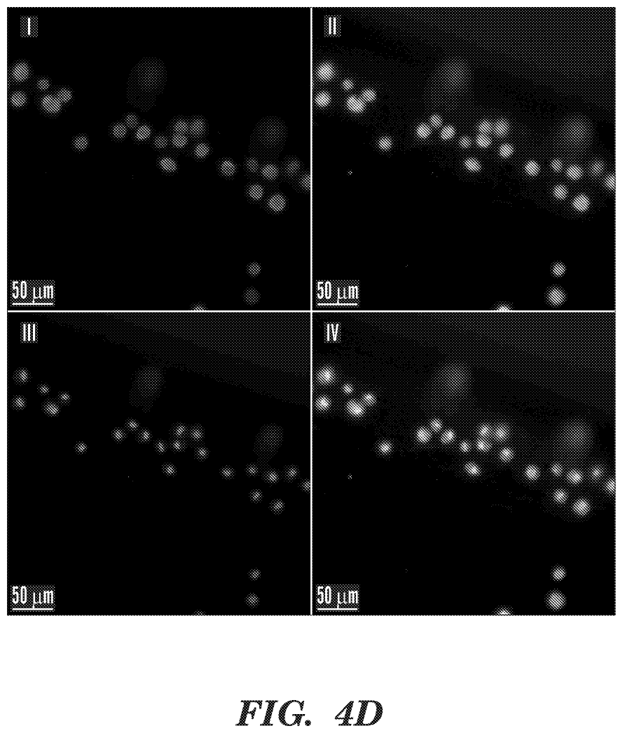

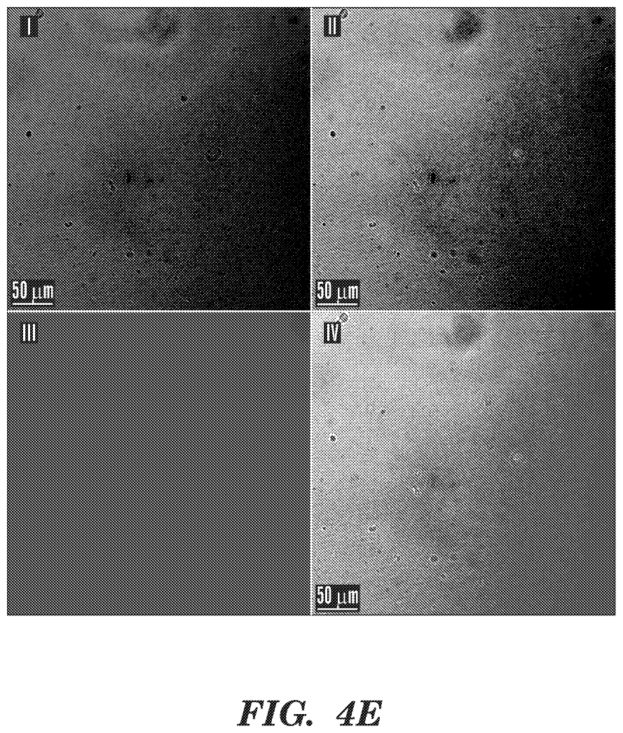

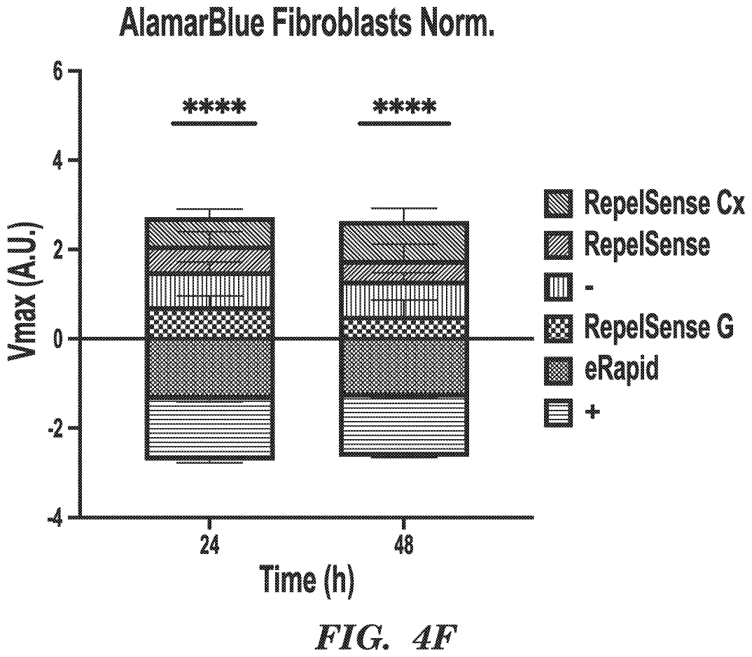

Given the integral role of fibroblasts in tissue regeneration and wound recovery, regulating fibroblast recruitment and adherence is crucial to mitigating foreign body response and improving the longevity and biocompatibility of implantable biosensors. [7] RepelSense's antifouling properties prevent fibrotic attachment and subsequent release of matrix proteins, without compromising cell metabolic function. In this study, fibroblasts from three donors were cultured on specific nanocomposite coatings and incubated at 37° C. across two time periods. Repel Sense coatings (RepelSense without antibiotics and RepelSense Cx) showed non-toxicity with cell counts akin to the negative control. However, RepelSense G had fewer cells (p<0.05), while eRapid and the cytotoxic control had significantly lower counts compared to the negative control (p<0.001) (see Supplementary files). Fibroblast extension, a pivotal marker for attachment, provides insights into biocompatibility and ensuing immunological response. [6,34] Microscopic analysis of RepelSense coatings (FIG. 4A & FIG. 4B) highlighted minimal fibroblast adhesion, preserving healthy nucleic sphericity, and underlining the safety of RepelSense. In contrast, the negative control displayed typical elongated fibroblast morphology with pronounced adhesions and fibrillar deposition (FIG. 4C), while the eRapid coating-treated cells appeared sparser, and the cytotoxic control showed no viable cells. [6] Importantly, the antifouling effect did not compromise cell metabolic activity, as verified by alamarBlue assay conducted immediately after incubation at 37° C. for 60 min, with background signal removed. Once again, the assay demonstrated increased metabolic activity (p<0.0001) in fibroblasts exposed to RepelSense coatings (RepelSense, RepelSense G, RepelSense Cx) compared to eRapid coating conditions for both 24 and 48 h incubation periods (FIG. 4F).

### Cytokine Production

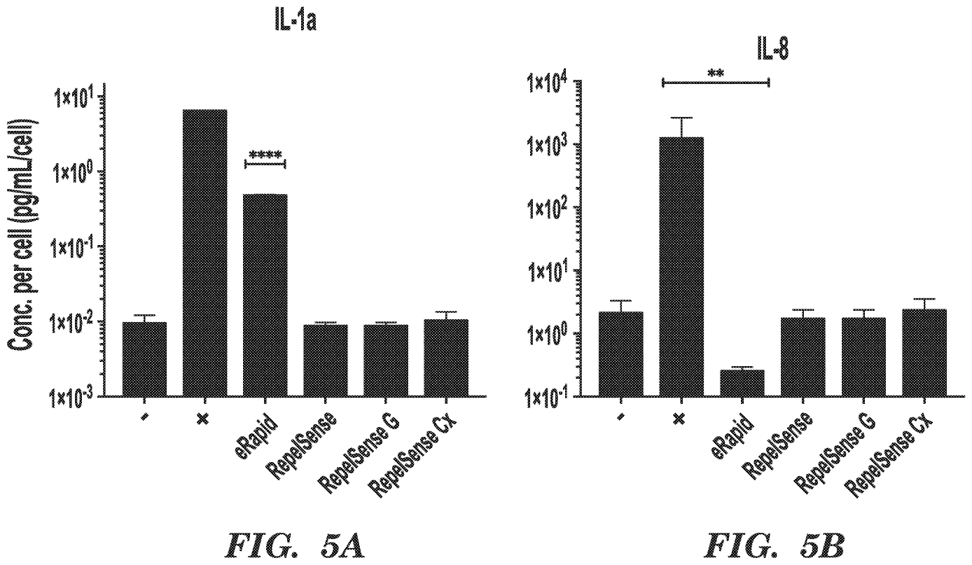

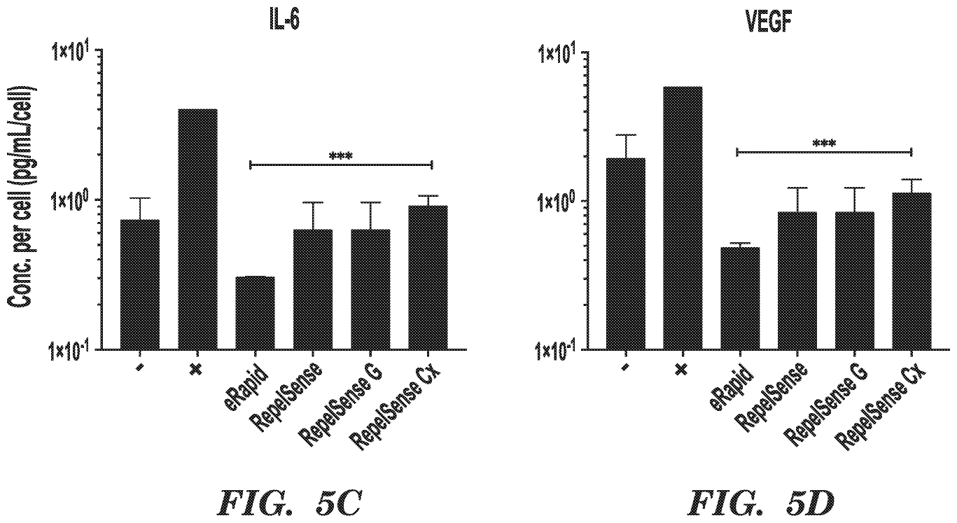

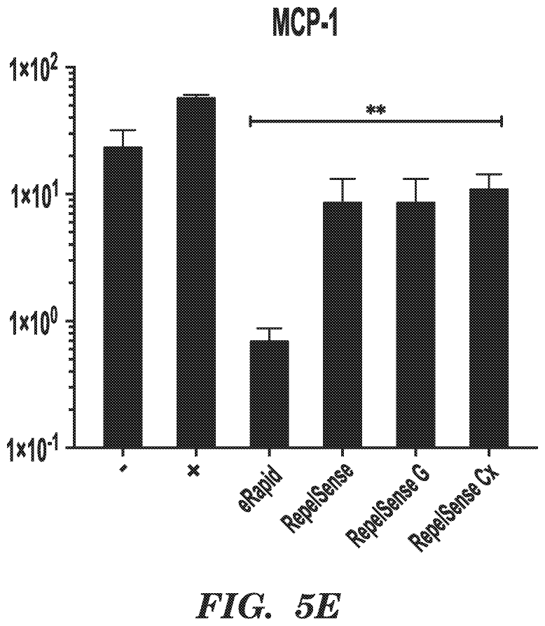

Monitoring cytokine expression is essential for optimal biomaterial design and functionality as high levels of pro-inflammatory cytokines, such as IL-6 and TNF-α, suggest potential material rejection, while anti-inflammatory cytokines like IL-10 indicate better material integration. [35,36] To discern the impact of RepelSense on inflammation, we analyzed the expression of key pro-and anti-inflammatory markers (IL-6, IL-1a, IL-10, IL-8, VEGF, MIP-1α, MCP-1, and TNF-α) in fibroblast supernatants at the 24-hour time point and results were normalized to cell counts. [35,36] For RepelSense, IL-1α, IL-8, IL-6, VEGF, and MCP-1 levels closely mirrored the negative control, suggesting a neutral cellular response which is often preferable for implanted devices (FIG. 5). [4,35] MIP-1α, IL-10, and TNF-α secretion were either absent or under the limit of detection (LoD) for all conditions. Compared to fibroblasts exposed to RepelSense, fibroblasts exposed to the eRapid coating condition revealed upregulated IL-1α expression (p<0.0001) and decreased but not significant expression of IL-8, IL-6, VEGF, and MCP−, and significant expression when compared to RepelSense Cx for IL-6 (p<0.001), VEGF (p<0.001), and MCP-1 (p<0.01). RepelSense's marked suppression of pro-inflammatory IL-1α and overall cytokine levels compared to the negative control underscore its non-inflammatory nature. However, the slight cytokine elevation in RepelSense Cx could suggest a reduced level of biocompatibility for Cx when compared to G as an antimicrobial component.

### Repelsense Maintains Electrochemical Sensitivity and Stability

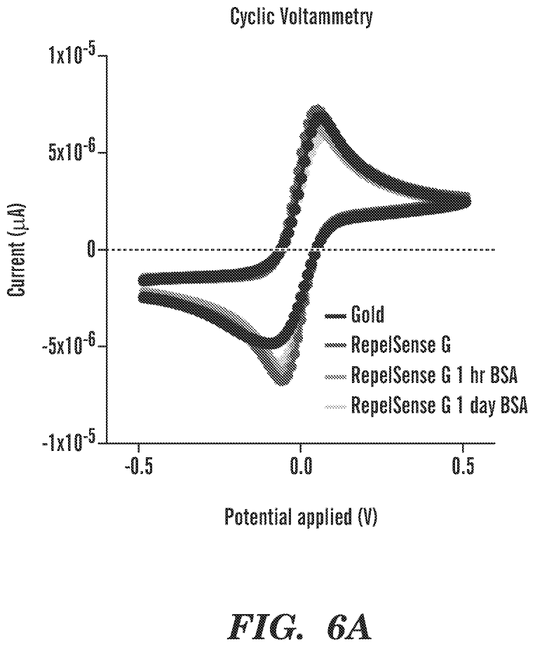

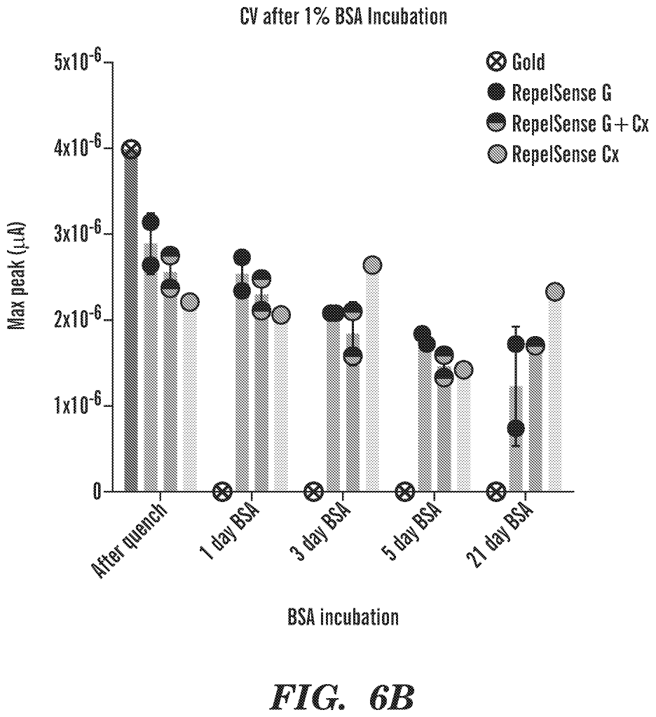

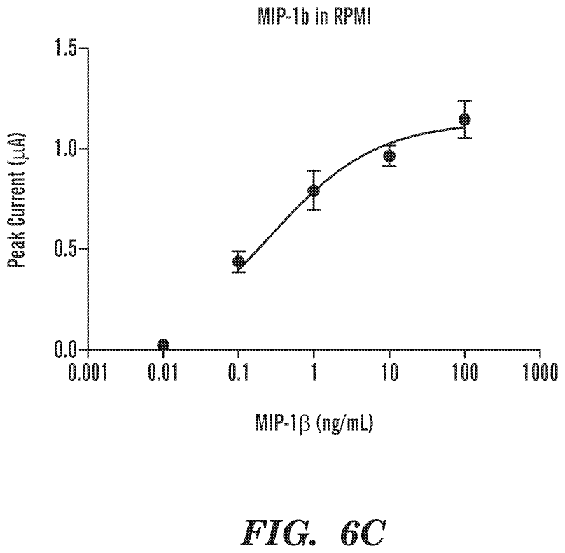

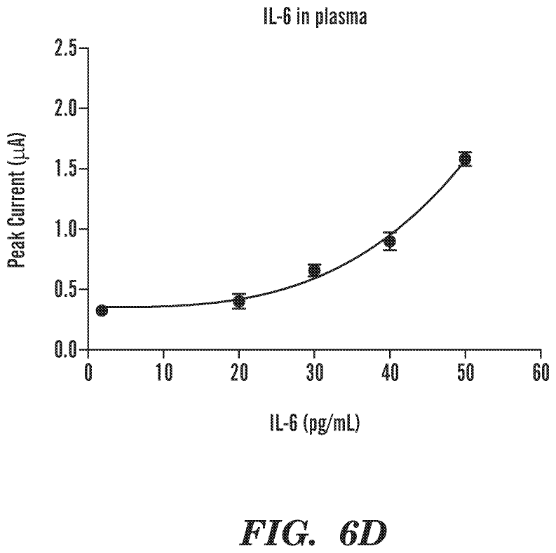

Electrochemical performance is key to achieving the sensor stability, longevity, and functionality required for long-term biosensing applications. [9] We verified the electrochemical performance and antifouling properties of RepelSense coatings with cyclic voltammetry (CV) by examining oxidation/reduction peaks and peak-to-peak distance (ΔEp) using potassium ferri/ferrocyanide. High current densities with oxidation/reduction peaks of 7.31/−7.22×10-6A were found after coating the electrode with RepelSense G, compared to the peaks of an uncoated gold electrode (6.79/−5.01×10-6A, respectively) (FIG. 6A). The antifouling capacity of each nanocomposite was then confirmed by exposing the electrodes to BSA, a typical blocking agent. High oxidation peaks of 6.48×10-6A, and 5.74×10-6A and reduction peaks of −6.71×10-6A and −6.17×10-6A were seen for RepelSense G after 1-h and 1-day exposure to 1% BSA, respectively, whereas the uncoated gold electrode was passivated immediately after BSA exposure. Excellent transfer kinetics facilitating electron flow while mitigating protein diffusion were further displayed by a ΔEp of 0.091 V, 0.091 V, and 0.1309 V for RepelSense G immediately after coating, 1-h, and 1-day in 1% BSA, which were below or on par with 0.1309 V for the uncoated gold electrode (FIG. 6A). All RepelSense coatings, including Repel Sense Cx and (RepelSense G+Cx) exposed to 1% BSA maintained remarkably high current densities and low ΔEps even after 21 days, demonstrating the ability of RepelSense coatings to maintain high signal strength and assay sensitivity with little evidence of sensor passivation, further confirming its utility for long-term sensing (FIG. 6B).

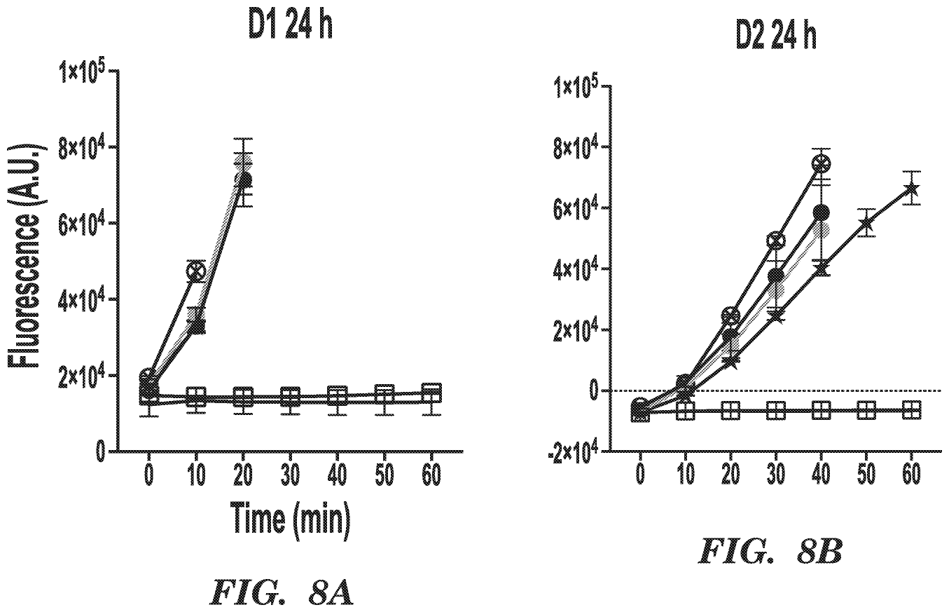

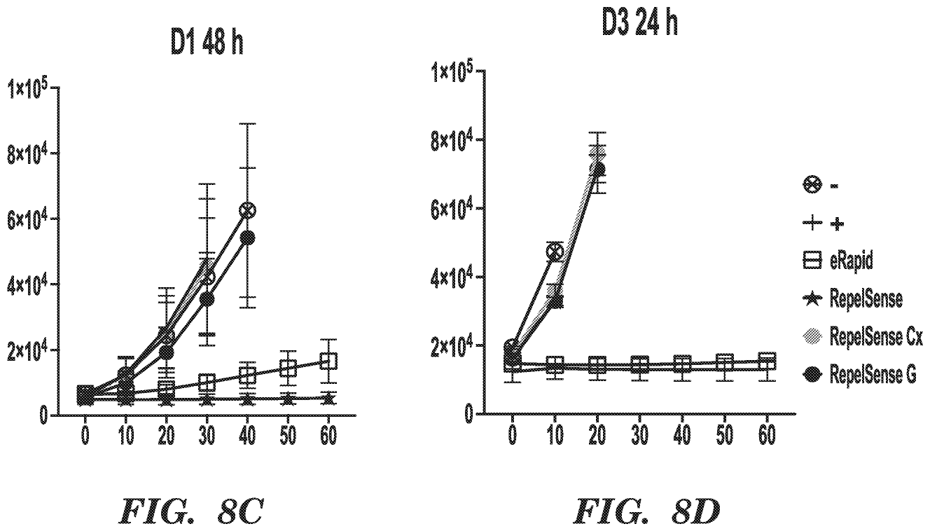

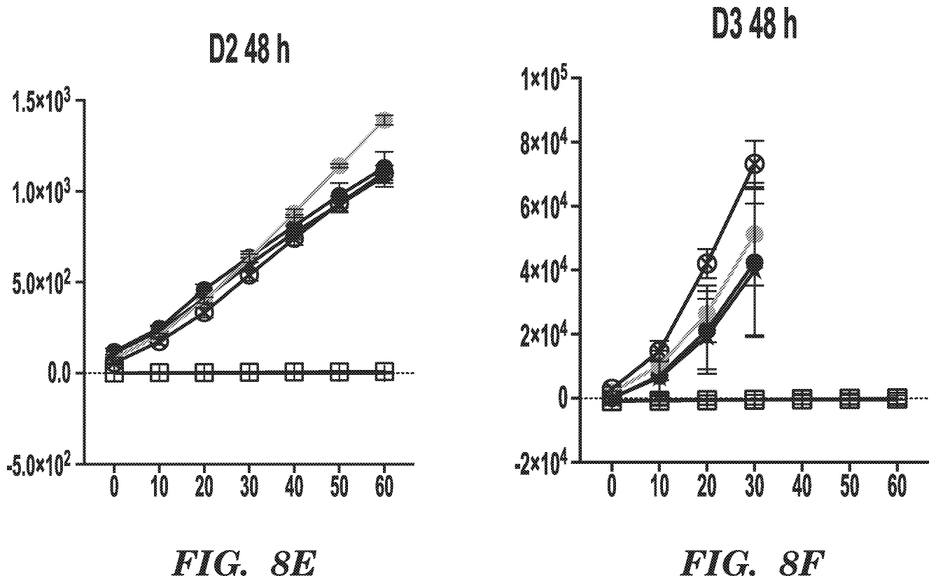

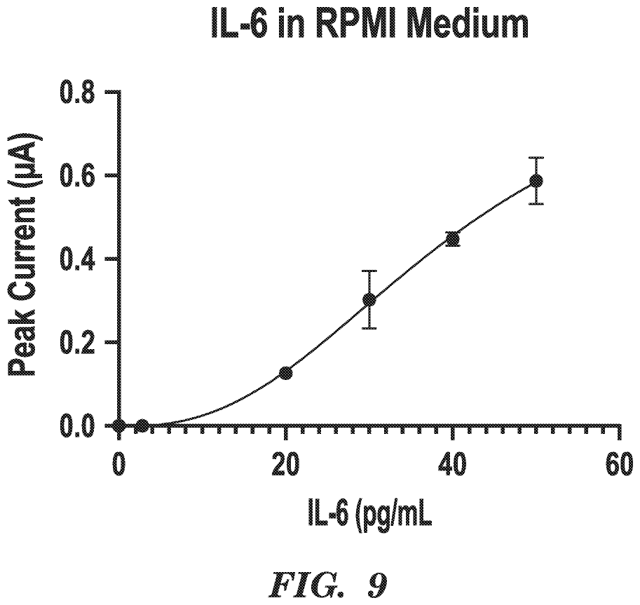

For further validation, the exceptional electrochemical properties of RepelSense G were leveraged to develop an immunosensor based on sandwich ELISA wherein the working electrodes were selectively functionalized with capture antibodies (cAb) by EDC/NHS chemistry. The cAb-immobilized immunosensor was exposed to different concentrations of target antigen, and detection was achieved using biotinylated secondary antibodies based on the reduction of poly-HRP-streptavidin binding followed by TMB precipitation. First, the potential of the RepelSense G-coated immunosensor to detect MIP-1b and IL-6 in a culture medium (RPMI) were explored. The immunosensor response was calibrated against different MIP-1b and IL-6 concentrations in the range of 0.10 to 100 ng mL−1 and 20 to 50 pg mL−1, respectively (FIG. 8). A detection limit of 0.096 ng mL−1 and 0.028 ng mL−1 was obtained for MIP-1b and IL-6 in RPMI, respectively. To evaluate immunosensing performance in complex biological samples, a RepelSense-coated immunosensor was calibrated using spiked concentrations of IL-6 in human plasma (FIG. 6) and obtained a LoD of 0.018 ng mL−1. This underlines the unique ability of RepelSense to maintain high sensor sensitivity even in complex biological samples.

### Conclusions

The rise of personalized medicine and digital health necessitates more stable implantable/wearable biosensors for continuous, long-term analyte monitoring. Compared with other implanted devices such as pacemakers and heart stents, biosensors experience considerably shorter lifetimes due to multiple failures in the complex, dynamic in vivo environment. [37] Addressing this, we successfully synthesized a new prototype using an cost-effective antimicrobial, antifouling, and biocompatible sensor coating formulation. This coating dually combats contamination by deterring microbial adhesion and neutralizing nearby microbial activity while remaining safe to mammalian cells. The adaptability and versatility of the RepelSense coating make it suitable for a wide range of sensing needs in the biomedical and industrial fields, such as next-generation wearables, implants, in vitro diagnostics, and bioreactors. While the intricate and gradual onset of FBR and biofilm formation limits the translation of in vitro data, our initial findings underline the immense potential of RepelSense to revolutionize the biocompatibility and functionality of both wearable and implantable biosensors.

Nevertheless, comprehensive testing and validation are imperative to mature this technology, such as clinical efficacy and safety evaluations. Gauging the antimicrobial effect of RepelSense against a wide range of microbes under physiologically relevant conditions such as flow and the presence of human blood is crucially important. Such evaluations will elucidate the combined impact of its antimicrobial and antifouling properties. Furthermore, assessing its biocompatibility through evaluations such as thrombogenicity and long-term stability is vital to ensure its effectiveness in vivo. Additionally, several sensor-related queries that still require in-depth electrochemical analysis using methods such as chronoamperometry, single-frequency electrochemical impedance spectroscopy, in-line sensing tests, and clinical accuracy employing clinical error grid analysis in vitro, are imperative to advance toward in vivo evaluations.

### Materials

### Coating Synthesis

### Antifouling Nanocomposite Preparation

The eRapid nanocomposite was prepared and drop-cast on custom-fabricated gold electrode chips (Telic Company, Santa Clarita, CA, USA) as previously described. [9,10] The antimicrobial, biocompatible nanocomposite (bovine serum albumin-genipin (BSA-GNP)) was similarly prepared by sonicating 8 mg mL−1 of pentaamine-modified graphene oxide (prGOx) nanoflakes (Millipore Sigma, no. 806 579) with 5 mg mL−1. BSA (IgG-Free, Protease-Free; Jackson ImmunoResearch, no. 001-000-162) in 10×10-3 m phosphate-buffered saline solution (PBS, pH 7.4) (Sigma-Aldrich, USA, no. D 8537). The solution mixture was sonicated by tip sonicator for 30 min using 1 s on/off cycles at 50% amplitude, 125 W and 20 kHz (Bransonic, CPX 3800) followed by heating (Labnet, no. D 1200) at 105° C. for 5 min to denature the protein. The resulting opaque black mixture was centrifuged at 16.1×g for 15 min to remove excess aggregates. The semitransparent nanocomposite supernatant solution was then mixed either with 70 % glutaraldehyde (Sigma-Aldrich, USA, no. G7776) for eRapid or GNP (Adooq Bioscience, USA, no. A11677) dissolved in 50 % ethanol at 1 mg mL−1 for RepelSense at a ratio of 69:1.

### Addition of Cross-linked Antibiotics

As a proof of concept, we first crosslinked antibiotics with primary amine groups to the BSA/prGOx nanocomposite to actively mitigate microbial adhesion and prevent the leaching of antibiotics. Specifically, we made various RepelSense coatings-gentamicin (G)-crosslinked nanocomposite, -ampicillin-crosslinked nanocomposite (A), and vancomycin-crosslinked nanocomposite (V) using different concentrations of each antibiotic. Briefly, gentamicin sulfate salt, ampicillin sodium salt, vancomycin hydrochloride, Cefepime (C), Ceftriaxone (Cx), and Colistin (Co) (all from Sigma-Aldrich) were dissolved in deionized (DI) water at 70 mg mL−1 and added to the prGOx/BSA/GNP coating, 1 μL per chip for a final concentration of 1 mg mL−1, or diluted further to 0.1 mg mL−1.

### Coating of Antifouling Nanocomposite Onto Electrodes

For each antifouling nanocomposite, 70 μL was drop-cast over the surface of gold electrodes that were pre-cleaned and plasma-treated as previously described[8], incubated in a humidity chamber overnight at room temperature, then rinsed and washed with PBS at 400 rpm for 2 min. Double-sided adhesive tape was used to create a well for liquids (ARseal 90 880 Polypropylene Double-Sided Adhesive Tape, Adhesive research Inc., Glen Rock, PA).

Chips were then exposed to 1 M ethanolamine (Sigma-Aldrich, USA, no. E9508) in PBS to quench the unreacted GTA and GNP groups before electrochemical analysis.

### Fabrication of Electrochemical Immunosensor

Chips with coated electrodes were exposed to a solution containing the GNP-G crosslinked prGOx/BSA nanocomposite overnight as described in the aforementioned section, to fabricate an electrochemical immunosensor. Chips were then washed in PBS at 400 rpm for 10 min and dried. Coated chips were functionalized with 400 mM EDC (Thermo Fischer Scientific) and 200 mM NHS (Sigma Aldrich) dissolved in 0.05 M MES, for 30 min, rinsed with DI water, and dried at RT. Three working electrodes were spotted with the optimized concentration of capture antibodies (anti-MIP (R&D systems; anti-IL-6 (Abcam)) diluted in PBS, and the fourth electrode was spotted with negative control (BSA) using an Xtend capillary microarray pin (LabNEXT) and all electrodes were incubated in a humidity chamber at 4° C. overnight. Chips were washed with PBS and treated with 1 M ethanolamine for 30 min to quench the unreacted GNP groups, followed by blocking with 2.5% BSA in PBS for 1 h. MIP antigen (R&D systems) was spiked into RPMI (RPMI 1640, Gibco, 72400-047) ranging from 0.01 to 100 ng mL−1 and chips were incubated with 15 IL of (MIP antigen+RPMI) samples for 1 h. Similarly, IL-6 antigen (Abcam) was spiked into RPMI or human plasma (Innovative Research, USA no. IPLAS-COV2P100UL), and chips were incubated with 15 μL of (IL-6 antigen+RPMI or human plasma) samples for 1 h.

Chips were rinsed with 0.05% PBST and treated with their respective biotinylated detection antibodies. Biotinylation of the MIP detection antibody was achieved using a Biotin Conjugation Kit (Abcam) and the IL-6 from ab246838 Human IL-6 Matched Antibody Pair Kit (Abcam). The MIP and IL-6 sensor was incubated with MIP DAb and IL-6 DAb for 15 and 30 min, respectively, and then washed with 0.05% PBST. DAb-treated sensors were exposed to 5 mg mL−1 of Poly-HRP-Streptavidin (Thermo Fisher Scientific) prepared in 0.1% BSA in PBST for 5 min and then rinsed with PBST. Precipitating TMB (Sigma Aldrich) was added to the MIP sensor for 1 min and to the IL-6 sensor for 3 min. Finally, sensors were rinsed with PBST, and electrochemical measurements were taken.

### Electrochemical Characterization of Antifouling Nanocomposite

Electrochemical characterizations were carried out as described previously. [9] Briefly, each chip contained four gold electrodes, a working electrode, and a reference electrode connected to the potentiostat (Autolab PGSTAT128N, Metrohm; VSP, Bio-Logic) through a connector built in-house. Electrochemical characterization of the antifouling nanocomposite was characterized by cyclic voltammograms (CV) in a redox aqueous solution of PBS containing 5×10-3 m [Fe(CN) 6]3−/4− at a scan rate of 1 V/s between −0.5 and 0.5 V. The antifouling capacity of the nanocomposite coating was tested after incubation with 1% BSA for 1 h and 24 h. The immunosensors were then characterized using CV in PBST at a scan rate of 1 V s-1 between −0.5 and 0.5 V. The on-chip integrated (uncoated) gold electrode was used as a quasi-reference electrode for all electrochemical measurements.

### Antimicrobial Efficacy Evaluation

### Coating of Microtiter Plate

The antimicrobial efficacy of each nanocomposite coating was evaluated using a broth dilution assay. Briefly, a Falcon™ 96-Well, Cell Culture-Treated, Flat-Bottom Microplate (Fisher Scientific) was coated with the respective combinations of nanocomposites in triplicate (43 μL per well) by the drop-casting method as described above, followed by incubation in a humidity chamber overnight at room temperature and washing with PBS. Coatings were then quenched by adding 1 M ethanolamine (6 μL ethanolamine to 94 μL PBS) for 30 min at room temperature.

### Inoculation With Bacteria

Escherichia coli(E. coli) Mar. 2, 1953, and Pseudomonas aerigunosa (P. aerigunosa) ATCC 10145 were cultured on LB agar plates. Fresh colonies were picked and placed in 5 mL LB medium at 37° C. for 8 h. This was then diluted at 1:500 to 1:1000 to inoculate the overnight culture. The overnight culture was then adjusted with the BD PhoenixSpec™ nephelometer to 0.5±0.05 MacFarland and then diluted to 1:20 in LB medium. To each treatment and control well, 100 μL LB medium was added, followed by 100 μL of the diluted bacterial suspension for a final concentration of ~2×10⁶ CFU mL−1 per well, verified by plating. Absorbance was measured every 30 min for 10-15 h under shaking at OD 600 and 37° C. using the Synergy H1 Hybrid Multi-Mode Reader (Agilent technologies, USA) with BioTek Gen5 software.

### Antimicrobial Efficacy and Leaching

To assess potential antibiotic leaching from the nanocomposite, we assessed chips with three nanocomposite coatings: 1.) without antibiotics (prGOx/BSA/GNP), 2.) with antibiotics containing a primary amine-containing functional group (prGOx/BSA/GNP/G), and 3.) an antibiotic with no primary amine groups (meropenum, prGOx/BSA/GNP/M). Initially, two pieces of double-sided tape were placed to create a well in which LB medium and bacterial suspension could remain in contact with the nanocomposite coating. Then 40 μL of ~102 CFU mL−1 P. aeruginosa in LB medium was added to the nanocomposite and incubated in a humidity chamber for 24 h on a shaker at 70 rpm and 37° C. The following day, the remaining inoculum was collected and the nanocomposite was washed with 60 μL LB medium, serially diluted, plated, and counted after 24 h incubation at 37° C. This procedure was then repeated daily for two consecutive days.

Collected media was added to a 5 mm piece of ø filter paper (Thermo Fisher Scientific) placed on an LB agar plate which had been swabbed with P. aeruginosa in LB medium to create a bacterial lawn. As a control, a separate plate was swabbed and 1 μL of 70 mg mL−1 G was diluted at 1:20 in LB medium, 2-fold serially diluted, and added to 5 mm pieces of ø filter paper on the bacterial lawn. Plates were then incubated for 24 h at 37° C. whereafter the zones of inhibition around the filter papers were analyzed.

### Biocompatibility Evaluation

### Coating of Wells:

Nanocomposite coatings of different combinations were tested: eRapid, RepelSense, RepelSense G, and RepelSense Cx. As a positive control, a polyurethane (PU) film containing 0.1% zinc diethyldithiocarbamate (ZDEC) (Hetano Research Institute, Food and Drug Safety Center, Japan) and an uncoated tissue culture plate as a negative control was used in accordance with ISO 10993-1:2018: Biological evaluation of medical devices. Briefly, 1 Falcon™ 96-Well, Cell Culture-Treated, Flat-Bottom Microplate (Thermo Fisher Scientific) per donor was coated using the drop-casting method described above with each nanocomposite combination.

### Monocyte Isolation and Culture

De-identified human patient-derived apheresis collars (a by-product of platelet isolation) were obtained from the Crimson Biomaterials Collection Core Facility under approval from the Institutional Review Board at Harvard University (#22470); informed written consent was not required. PBMCs were isolated by density centrifugation using Lymphoprep (StemCell Technologies, 07801), and magnetic beads were used for negative selection of monocytes (StemCell Technologies, 19058). Monocytes were then seeded at approximately 100,000 cells per well and cultured under the various experimental conditions in complete RPMI, 10% Fetal bovine Serum (FBS, Gibco, 10082-147), and 1% Penicillin/Streptomycin (Gibco, 15140122). Supernatant from each condition and timepoint was then collected and stored at −20° C. for MSD analysis; live cells were harvested and analyzed by flow cytometry, alamarBlue™ Cell Viability assay, MDS analysis, and microscopy.

### Fibroblast Isolation and Culture

Full-thickness samples were isolated anonymously from healthy regions of colon resections and processed in the Department of Pathology at Massachusetts General Hospital under an existing Institutional Review Board (approved protocol number is #2015P001859). All samples were pooled into one sample pool. Deidentified samples (macroscopically grossly unaffected regions of the colon) were collected from adult patients undergoing endoscopy for abnormal complaints. Collected samples were digested with 2 mg mL−1collagenase I (17100-017; Thermo Fisher Scientific, Waltham, MA) supplemented with 10 mmol L-1 Y-27632(Y0503; Sigma-Aldrich, St. Louis, MO) for 40 min at 37° C. with intermittent agitation, then suspended in fibroblast growth medium (FGM-2, Lonza, CC-3131) supplemented with a bullet kit (Lonza, Cat #CC-4126) for further expansion. Culture medium was changed every other day to provide enough nutrients for proliferation. Cells were seeded on the nanocomposite-coated 96 well plates, allowed to reach 70% confluency, and incubated for 24 and 48 h at 37° C. Supernatant from each condition was then collected.

### Flow Cytometry

Cells for flow cytometry analysis were harvested via pipetting, centrifuged at 300×g for 5 min, and resuspended in PBS for staining. Cells were first labeled with ViaKrome fixable viability dye (1:150 dilution; Beckman Coulter, C36628), followed by a 30 min incubation with fluorophore-labeled antibodies and Fc Block (1:100 dilution) at 4° C. Cells were washed twice with PBS and then fixed with Cytofix (BD Biosciences, 554655) for 15 min at 4° C. Cells were centrifuged and re-suspended in PBS containing 2% FBS, 2 mM EDTA and stored at 4° C. prior to analysis using a Cytoflex LX flow cytometer (Beckman Coulter). Results were analyzed using FlowJo V10 software (Flowjo, LLC). Antibodies to CD80 (#305232), CD40 (#334345), HLA-DR (#361610), CD1c (#331526), and CD86 (#305430) were obtained from Biolegend; antibodies to CD83 (#19677) were obtained from Santa Cruz Biotechnology; antibodies to CD14 (#563561) were obtained from BD.

### Alamarblue Viability Assay

To assess cell metabolic activity, monocytes and fibroblasts were treated with alamarBlue™ Cell Viability Reagent (ThermoFisher). Cells were washed and resuspended in fresh RPMI with alamarBlue at 10% total volume, and incubated at 37° C. Fluorescence was measured using Biotek fluorescence spectrophotometry at an excitation wavelength of 560 nm and an emission wavelength of 590 nm. Measurements were taken every 10 min for 1 h.

### MSD Cytokine/chemokine Analysis

Cytokines and chemokines from monocytes and fibroblasts were analyzed using the Meso Scale Discovery MSD® U-PLEX development pack (Meso Scale Discovery, Maryland, USA) with spots for IL-1, IL-6, IL-10, IL-8, VEGF, MIP-1a, MCP-1, and TNF-a according to the manufacturer's protocol. Briefly, antibody solutions were created by diluting each biotinylated antibody to 10 μg mL−1, then added to 300 μL of the assigned linker, vortexed, and incubated at room temperature for 30 min. Then 200 μL of stop solution was added, followed by vortexing and incubation for another 30 min; antibodies were then pooled. Coating solution was added at 50 μL per well, sealed, and incubated at room temperature for 1 h, followed by washing 3 times with PBS-T. To each well, 50 μL of sample, calibrator, or control was added and incubated for 1 h followed by washing. Detection antibody was then added at 50 μL per well, incubated for 1 h, washed, and read on the MESO QuickPlex SQ 120 (Meso Scale Discovery).

### Immunofluorescent Staining

After washing cells with PBS to remove non-attached cells, samples were fixed at room temperature for 15 min with 4% paraformaldehyde. Cells were then permeabilized and blocked with 5% BSA and 0.1% TritonX-100 for 1 h at room temperature. Smooth alpha muscle antibody (a-SMA, Abcam, ab5694), was diluted 1:200 in 2% BSA and incubated for 1 h at room temperature. After washing the samples, secondary antibodies (anti-rabbit, 488) and Phalloidin (647, Abcam, ab176759) were added in 2 % BSA at 1:200 dilution and incubated at room temperature for 1 h. Finally, samples were stained for nuclei using Hoechst diluted in DPBS at 1:1000. Samples were imaged using the Zeiss Axiovert 40 C-FL ready 1 microscope, and three representative images for each condition were taken at 60× and analyzed using Imaris x64.9.7.2 with the threshold recommended for each channel.

### Statistics

Results were expressed as mean values±standard deviation (SD), with differences as determined by ANOVA for analysis of variance with Dunnett's multiple comparison test. The results were considered significant when displaying P-values <0.05 and are indicated by asterisks, *: p<0.05 or **: p<0.01, *: p<0.001, *p<=0.0001.

### REFERENCES

[1] M. J. Russo, M. Han, P. E. Desroches, C. S. Manasa, J. Dennaoui, A. F. Quigley, R. M. I. Kapsa, S. E. Moulton, R. M. Guijt, G. W. Greene, S. M. Silva, ACS Sens 2021, 6, 1482.

[2] E. E. M. Van Den Bosch, N. H. M. De Bont, J. Qiu, O.-J. Gelling, A Promising Solution to Enhance the Sensocompatibility of Biosensors in Continuous Glucose Monitoring Systems, Vol. 7, 2013.

[3] P. Vergidis, R. Patel, Infect Dis Clin North Am 2012, 26, 173.

[4] C. R. Arciola, D. Campoccia, L. Montanaro, Nature Reviews Microbiology 2018 16:72018, 16, 397

[5] S. Seget, E. Rusak, M. Partyka, E. Samulska, A. Pyziak-Skupień, H. Kamińska, E. Skała-Zamorowska, P. Jarosz-Chobot, Acta Diabeto/2021, 58, 191.

[6] D. Zhang, Q. Chen, C. Shi, M. Chen, K. Ma, J. Wan, R. Liu, 2020, 2007226, 1.

[7] Y. Wang, S. Vaddiraju, B. Gu, F. Papadimitrakopoulos, D. J. Burgess, J Diabetes Sci Technol 2015, 9, 966.

[8] J. Sabaté del Río, O. Y. F. Henry, P. Jolly, D. E. Ingber, Nat Nanotechnol 2019, 14,1143.

[9] S. S. Timilsina, N. Durr, M. Yafia, H. Sallum, P. Jolly, E. Donald, 2021, 1.

[10] U. Zupančič, P. Jolly, P. Estrela, D. Moschou, D. E. Ingber, Adv Funct Mater 2021, 31,1.

[11] S. A. Poursamar, A. N. Lehner, M. Azami, S. Ebrahimi-Barough, A. Samadikuchaksaraei, A. P. M. Antunes, Mater Sci Eng C Mater Biol Appl 2016, 63, 1.

[12] D. Utami Nike, N. Izzah Md Fadilah, N. Sallehuddin, A. Yasser Hamdi Nor Azlan, F. Hani Imran, M. Maarof, M. Busra Fauzi.

[13] H. Koo, R. N. Allan, R. P. Howlin, P. Stoodley, L. Hall-Stoodley, Nat Rev Microbiol 2017, 15, 740

[14] G. Zeng, R. Ogaki, R. L. Meyer, Acta Biomater 2015, 24, 64.

[15] S. Mahira, A. Jain, W. Khan, A. J. Domb, Antimicrobial Materials—An Overview, 2019.

[16] K. Bush, Ann N Y Acad Sci 2013, 1277, 5.

[17] D. Sun, M. Babar Shahzad, M. Li, G. Wang, D. Xu, Materials Technology 2015, 30, B90.

[18] J. A. Del Olmo, L. Ruiz-Rubio, L. Pérez-Alvarez, V. Sáez-Martínez, J. L. Vilas-Vilela, Antibacterial coatings for improving the performance of biomaterials, Vol. 10, MDPI AG, 2020.

[19] J. C. Tiller, C. J. Liao, K. Lewis, A. M. Klibanov, Proc Natl Acad Sci U S A 2001, 98, 5981.

[20] C. Adlhart, J. Verran, N. F. Azevedo, H. Olmez, M. M. Keinänen-Toivola, I. Gouveia, L. F. Melo, F. Crijns, Journal of Hospital Infection 2018, 99, 239.

[21] P. Carvalho, P. Sampaio, S. Azevedo, C. Vaz, J. P. Espinós, V. Teixeira, J. O. Carneiro, Appl Surf Sci 2014, 307, 548.

[22] J. Sabaté del Río, O. Y. F. Henry, P. Jolly, D. E. Ingber, An antifouling coating that enables affinity-based electrochemical biosensing in complex biological fluids, Vol. 14, 2019, pp. 1143-1149.

[23] B. D. Saboo, P. A. Talaviya, Indian J Endocrinol Metab 2012, 16, S259.

[24] U. Klueh, J. T. Frailey, Y. Qiao, O. Antar, D. L. Kreutzer, Biomaterials 2014, 35, 3145.

[25] R. Cleeland, E. Squires, Am J Med 1984, 77, 3.

[26] G. F. Dall, S. T. J. Tsang, P. J. Gwynne, S. P. Mackenzie, A. H. R. W. Simpson, S. J. Breusch, M. P. Gallagher, J Antimicrob Chemother 2018, 73, 1830.

[27] A. O. Elzoghby, Journal of Controlled Release 2013, 172, 1075.

[28] A. Lescoat, V. Lecureur, M. Roussel, B. L. Sunnaram, A. Ballerie, G. Coiffier, S. Jouneau, O. Fardel, T. Fest, P. Jégo, Clin Rheumatol 2017, 36, 1649.

[29] L. Ziegler-Heitbrock, J Leukoc Biol 2007, 81, 584.

[30] G. Monneret, F. Venet, Crit Care 2014, 18, 102.

[31] A. Palojärvi, J. Petäjä, S. Siitonen, C. Janér, S. Andersson, Pediatric Research 2013 73:1 2012, 73, 469.

[32] Z. Li, X. Ju, P. A. Silveira, E. Abadir, W. H. Hsu, D. N. J. Hart, G. J. Clark, Front Immunol 2019, 10, 1312.

[33] X. Ju, P. A. Silveira, W.-H. Hsu, Z. Elgundi, R. Alingcastre, N. D. Verma, P. D. Fromm, J. L. Hsu, C. Bryant, Z. Li, F. Kupresanin, T.-H. Lo, C. Clarke, K. Lee, H. McGuire, B. Fazekas de St. Groth, S. R. Larsen, J. Gibson, K. F. Bradstock, G. J. Clark, D. N. J. Hart, J Immunol 2016, 197, 4613.

[34] B. Hinz, G. Celetta, J. J. Tomasek, G. Gabbiani, C. Chaponnier, Alpha-Smooth Muscle Actin Expression Upregulates Fibroblast Contractile Activity, Vol. 12, 2001.

[35] S. Franz, S. Rammelt, D. Scharnweber, J. C. Simon, Immune responses to implants—A review of the implications for the design of immunomodulatory biomaterials, Vol. 32, 2011, pp. 6692-6709.

[36] M. Scatena, K. V. Eaton, M. F. Jackson, S. A. Lund, C. M. Giachelli, Macrophages: The bad, the ugly, and the good in the inflammatory response to biomaterials, 2016.

[37] J. Xu, H. Lee, Chemosensors 2020, Vol. 8, Page 66 2020, 8, 66.

### EXAMPLE 2

Medical devices that contain implantable or wearable biosensors remain limited by their short lifespan due to passivation and electrode deterioration, adverse immune responses from tissue trauma during implantation/insertion, encapsulation responses that alter analyte accessibility and the surface characteristics of the sensors, and a physiological mismatch between the sensor and surrounding tissue. In addition, non-specific absorption can lead to the attachment of microbes and biofilm formation, severely limiting the lifetime of the sensor. Here, is described a new formulation of the thin-film nanocomposite electroconductive coating that advances previous eRAPID design and exhibits antimicrobial activity while retaining outstanding antifouling properties, high electroconductivity, and excellent electrochemical behavior, which should have great value for increasing the lifespan and functionality of implantable and wearable medical devices. Specifically, it is demonstrated that application of this coating inhibits microbial attachments and proliferation on electrochemical sensors and limits the non-specific binding of proteins that might passivate the sensitivity and stability of the sensor. Moreover, the biocompatibility of the biosensor is improved by replacing cytotoxic components with non-toxic alternatives.

With global trends in a growing geriatric population, electrochemical biosensors are a cornerstone of precision medicine and the future of health care with invaluable advantages over other sensing technologies. Recent advancements in electronics and microfabrication have promoted the development of implantable biosensors, offering continuous real-time sensing of biochemical markers to diagnose, monitor, manage, and treat various diseases (e. g, diabetes).

Two main challenges hinder the implementation and long-term use of implantable biosensors: 1) Biofouling (non-specific cell/protein absorption) of biomolecules present in biological fluids or tissues can promote electrode deterioration and passivation, and thereby reduce electrochemical performance (sensitivity/specificity) and the longevity of the sensor⁽¹⁾. Non-specific binding of proteins to the sensor surface also leads to microbial chemotaxis, adhesion, and biofilm formation, increasing infection risks and implant failures⁽²⁾; 2) Inflammatory responses may arise from the implantation process, the physiological mismatch between the mechanical moduli of the electrode and surrounding tissue, and other immunogenic implant constituents/surface characteristics. In addition, the development of a foreign body response (FBR) may lead to fibrous capsule formation surrounding the device, further compromising sensor functionality.

Numerous technologies address this challenge by utilizing antifouling coatings⁽³⁾. While many of the techniques employed may prevent non-specific binding, few actively utilize antimicrobial components to secure a biostatic environment or use downright cytotoxic techniques⁽⁴⁾ and their electroconductivity properties are often poor.

### Results

Described herein are further advancements to the eRAPID nanocomposite (NC) coating for electrochemical sensors. The eRAPID NC coating consists of a matrix of bovine serum albumin (BSA) and reduced graphene oxide (rGOx) cross-linked with glutaraldehyde (GTA) (NC-BSA/rGOx). Herein is an improved coating that improves the sensor's antimicrobial and antifouling properties, as well as its biocompatibility^{(5,6)}, while retaining the other desirable features of the electroconductive eRAPID coating.

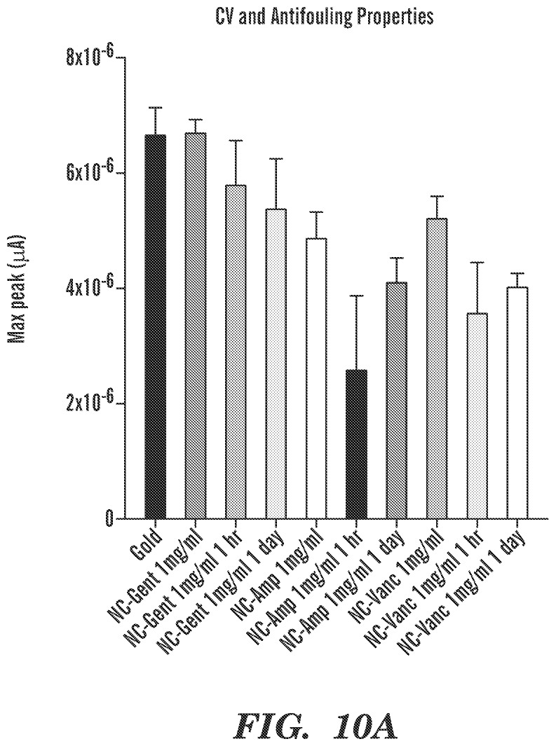

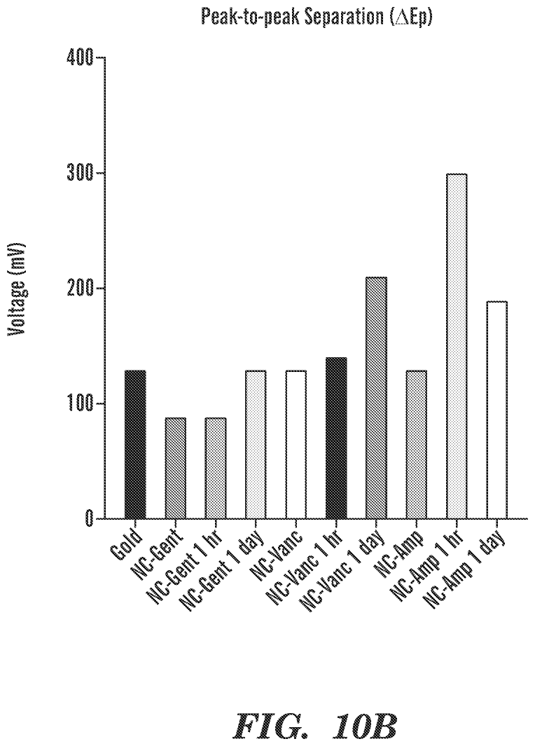

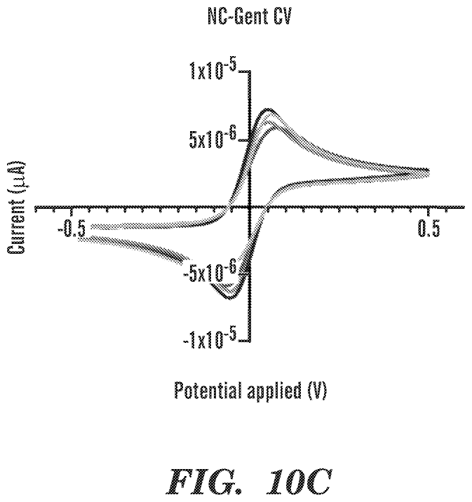

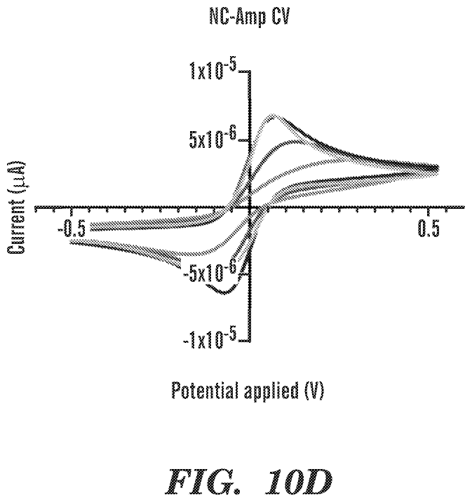

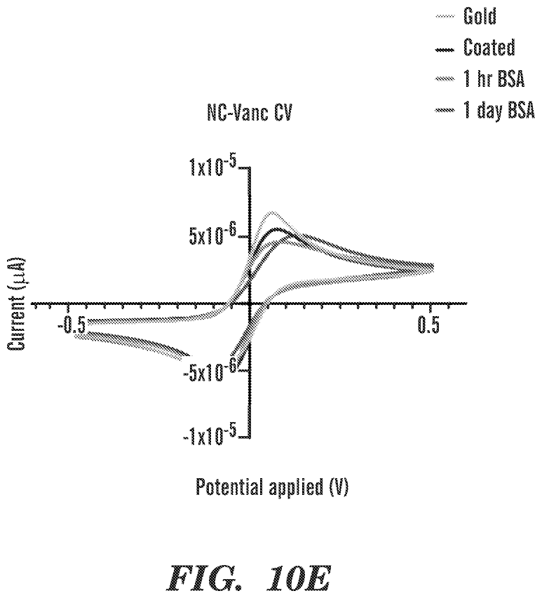

As a proof of concept, the inventors first incorporated antibiotics with amine groups crosslinked to the coating to mitigate microbial adhesion actively and prevent the leaching of antibiotics into the sample as seen in FIG. 10. Specifically, the inventors made a gentamicin-crosslinked nanocomposite (NC-gent), ampicillin-crosslinked nanocomposite (NC-amp), and vancomycin-crosslinked nanocomposite (NC-vanc) using different concentrations of antibiotic. Briefly, gentamicin sulfate salt, ampicillin sodium salt, and vancomycin hydrochloride (Sigma-Aldrich) were dissolved in DI water at 70 mg/ml and added to the coating 1 μl per chip for a final concentration of 1 mg/ml or diluted further to 0.1 mg/ml. Next, the electrochemical performance and antifouling properties of the NCs were tested using cyclic voltammetry (CV) after quenching the coating for 30 minutes with ethanolamine followed by 1 hour and 1 day of incubation with 1% BSA. These were compared to an uncoated electrode (gold). As seen in FIG. 2, the antibiotic-crosslinked NCs maintained remarkably high current densities and low ΔEps even after a 1-day incubation with 1% BSA demonstrating retention of excellent electrochemical characteristics, which is key to fabricating highly sensitive biosensors.

In addition, the inventors created a version of this coating using antimicrobial nanoparticles. Zinc oxide nanoparticles (ZnO NPs) (<18 nm) and silver nanoparticles (Ag NPs) (30-50 nm) (US Research Nanomaterials Inc.) were added to the nanocomposite coating at 3.2 and 5 mg/ml, respectively. The particles were each dispersed in PBS using sonication for 1 hour and then added to the BSA/rGOx NC and placed in a sonication bath for an additional 30 minutes. The coatings were otherwise prepared as usual using the rapid drop-casting method, which consists of heating on a hotplate at 85° C. for 30 seconds (rapid), or incubated at room temperature overnight (overnight).

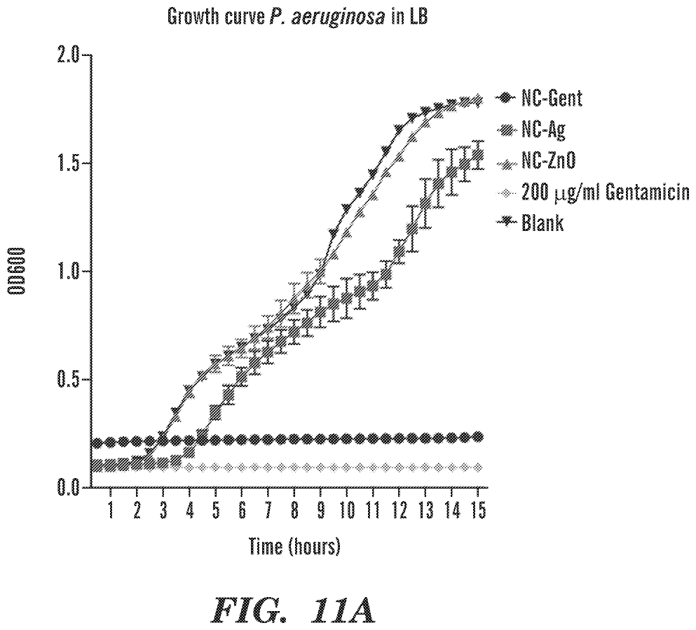

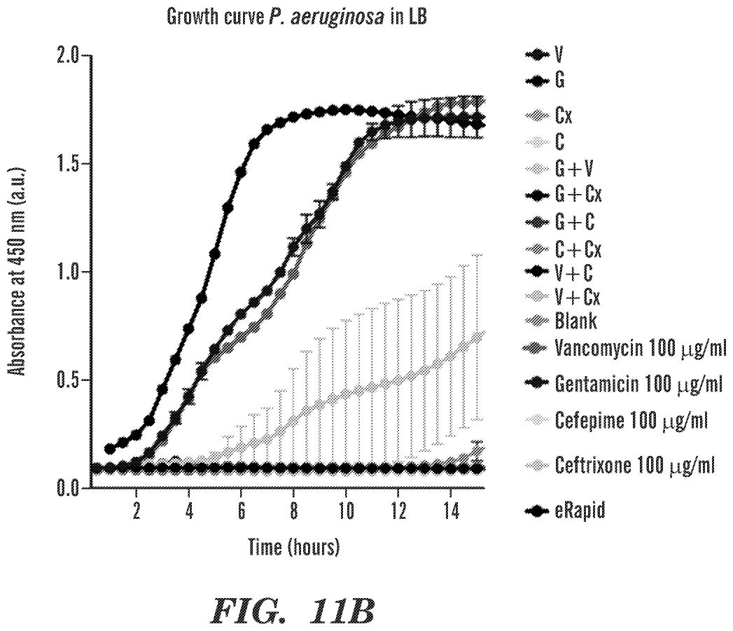

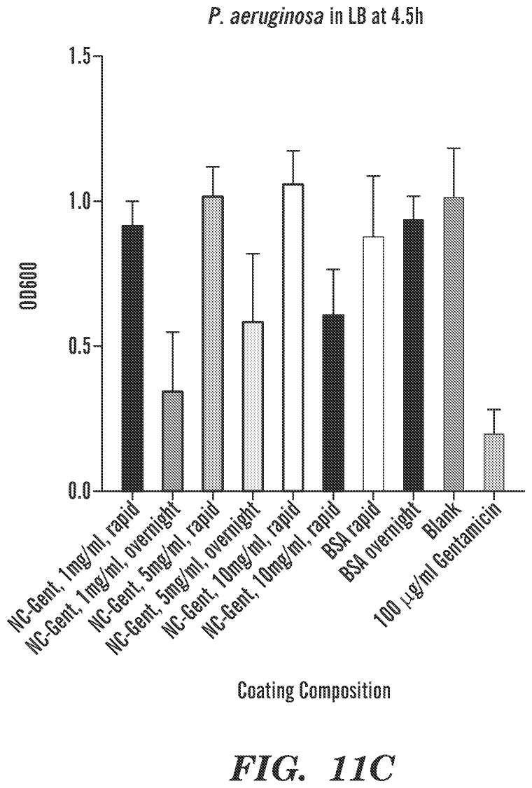

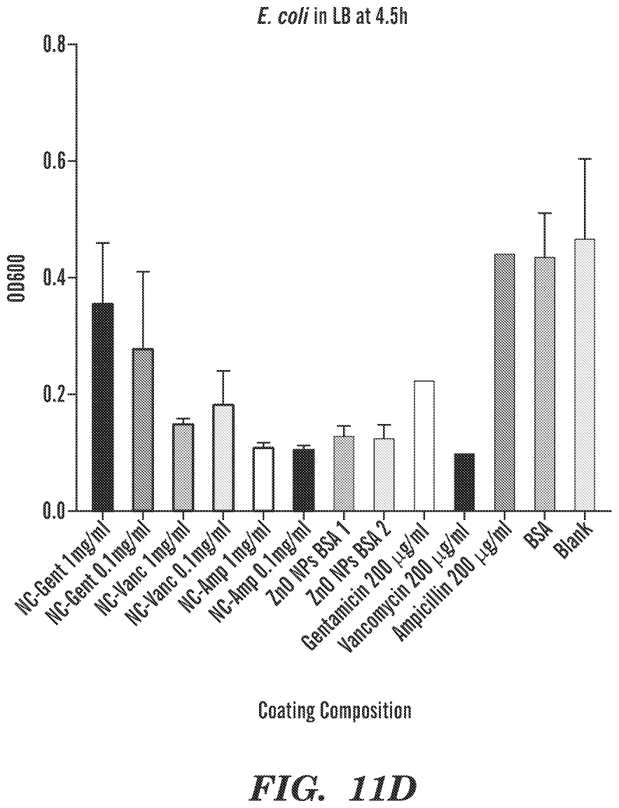

The inventors then tested the antimicrobial efficacy of these novel coatings of the ZnO NP nanocomposite (NC-ZnO) and Ag Np nanocomposite (NC-Ag) using a broth microdilution assay. Briefly, a 96-well plate was coated at the well bottom with various versions of the antibiotic crosslinked nanocomposite. Escherichia coli and Pseudomonas aeruginosa bacteria cultivated to logarithmic phase at 37° C. in LB media were adjusted to 0.5±0.05 MacFarland, diluted 1:20 and added 100 μl to each well followed by 100 μl LB medium for a final concentration of ~2×10⁶ CFU/mL, verified by plating. The plate was incubated at 37° C. in a plate reader under shaking for 10 hr with optical density (OD) measurements taken every 30 min at 600 nm. The mid-logarithmic growth phase was determined at the OD values for each treatment group averaged and compared as seen in FIGS. 11A-11D. Surprisingly, the coatings containing antibiotics inhibited growth compared to the control.

Secondly, the inventors improved the coating biocompatibility coating by replacing the glutaraldehyde crosslinker with genipin (GNP), a natural crosslinking low-toxic agent. GNP (Adooq Bioscience) was dissolved in 1 mg/ml 50% ethanol and added at a 1:69 ratio to the BSA/rGOx nanocomposite. 200 μl of monocytes isolated from PBMC in RPMi were seeded monocyte onto a 96-well plate coated with the original BSA/rGOx/GTA-crosslinked coating and the novel GNP crosslinked coating. In addition, a version with recombinant human serum albumin (HSA) was included. As a positive control, a polyether urethane (PU) film containing 0.1% zinc diethyldithiocarbamate (ZDEC) and a high-density polyurethane film as a negative control was used in accordance with ISO 10993-1:2018—Biological evaluation of medical devices. The cells were incubated at 37° C., 5% CO2 for 24 hours. After 24 h incubation, some cells were harvested, replicates pooled, and surface markers stained using fluorescence-activated cell sorting (FACS) staining followed by flow cytometry analysis. The remaining cells were treated with alamarBlue to assess cell metabolic activity. Finally, cells were washed and resuspended in a fresh cell culture medium with alamarBlue equal to 10% of the total volume. The cells were measured using fluorescence spectrophotometry at an excitation wavelength of 560 nm and an emission wavelength of 590 nm.

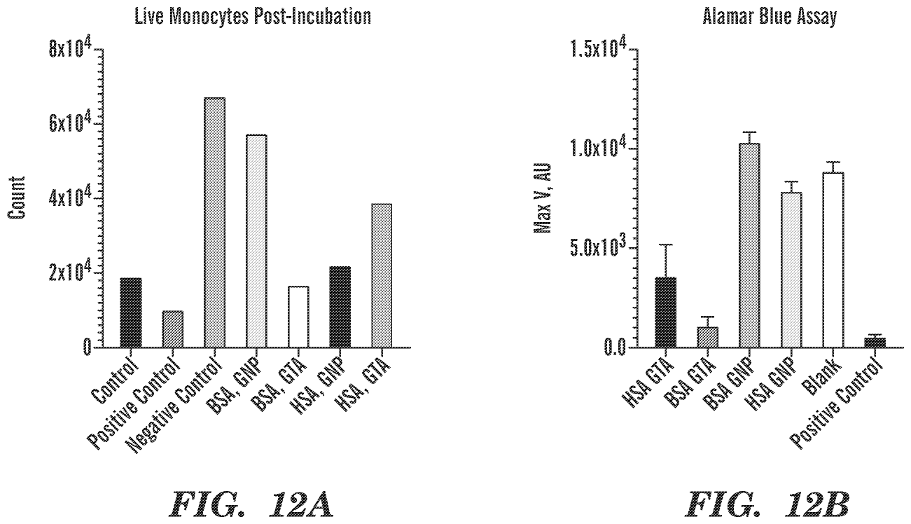

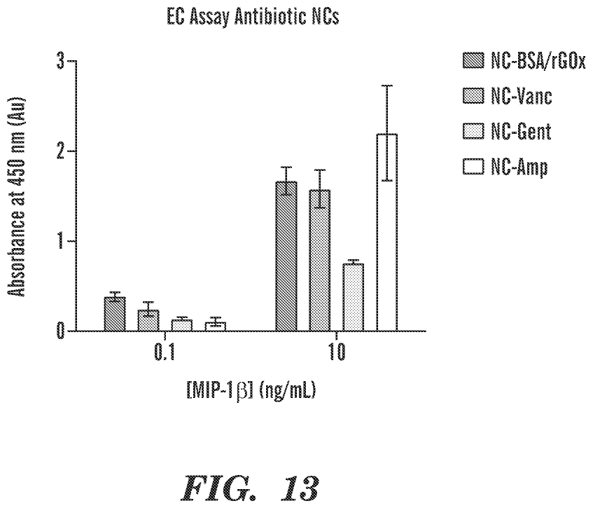

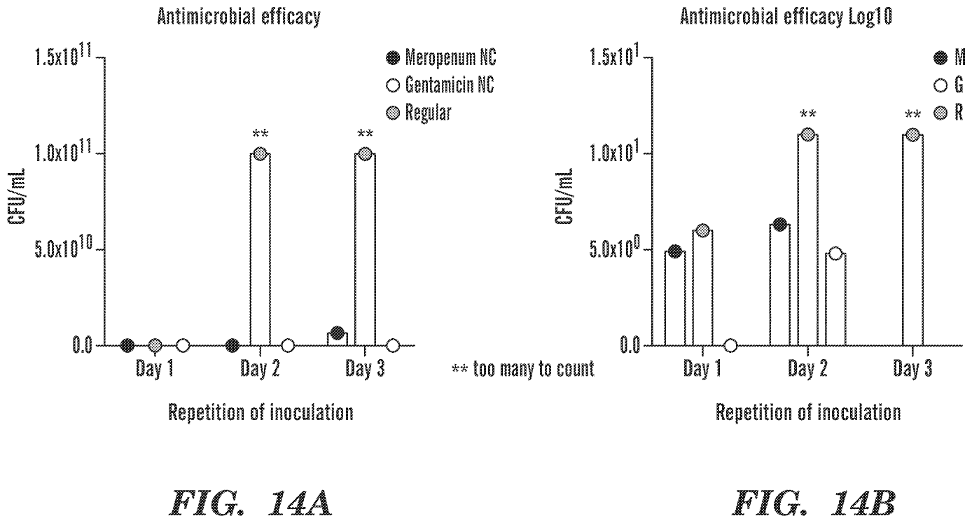

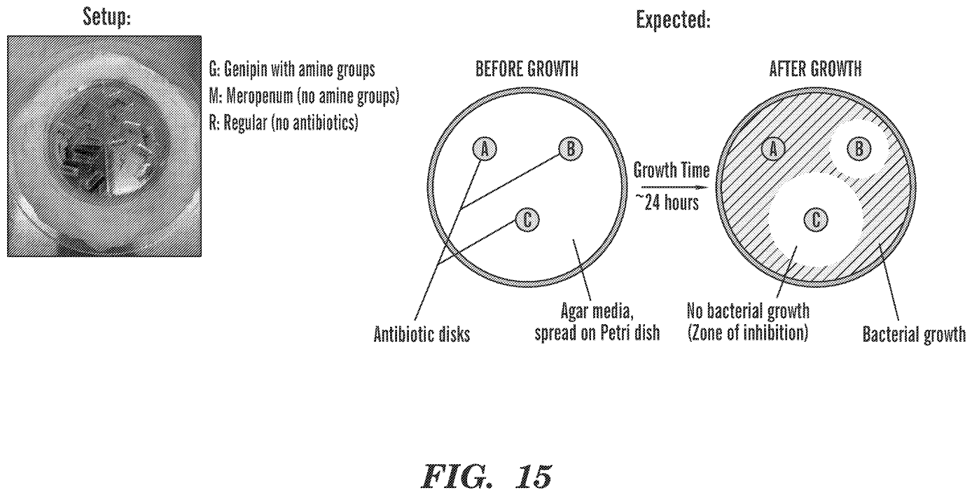

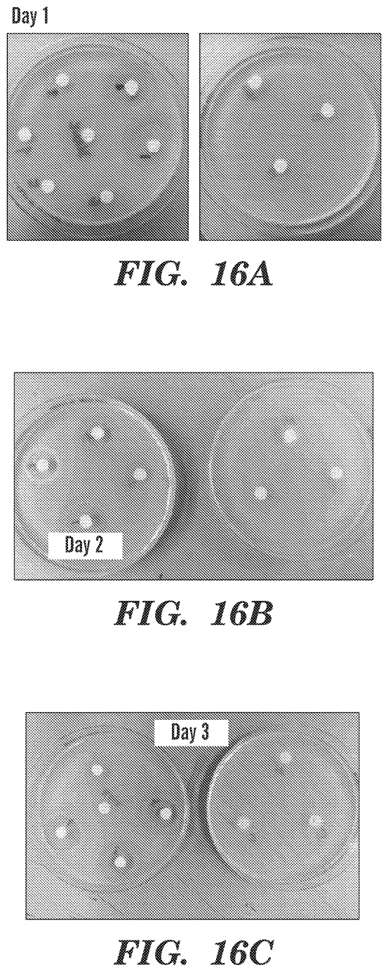

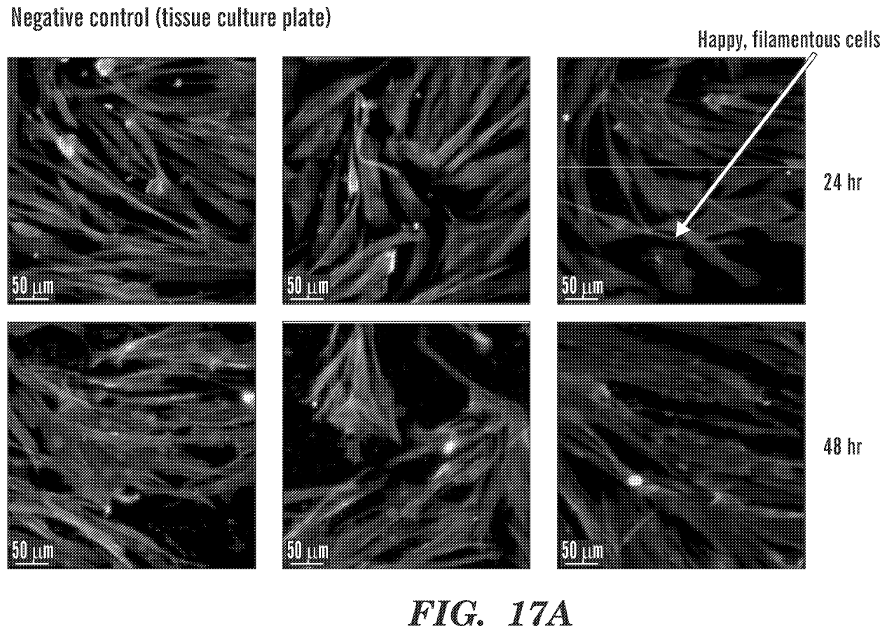

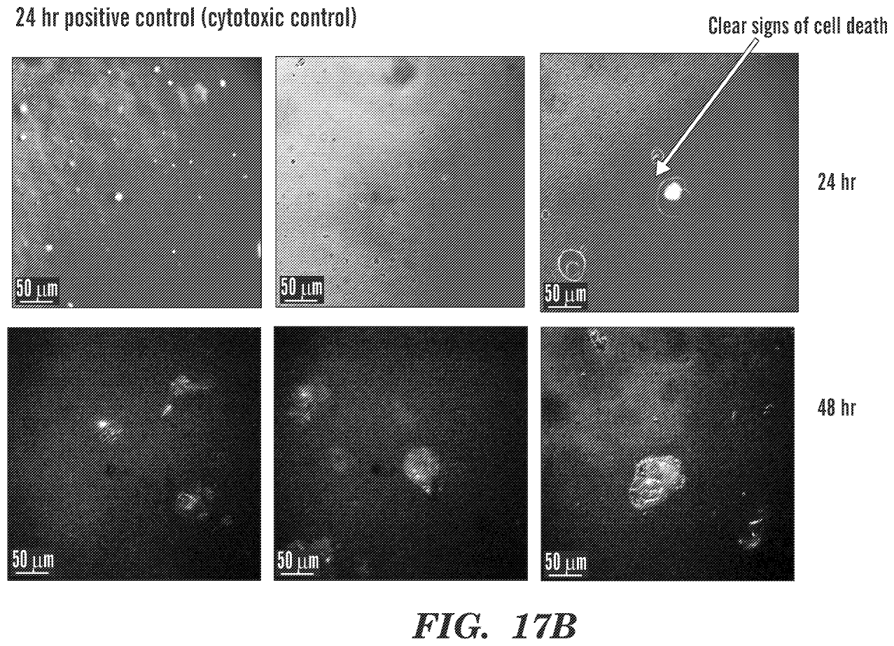

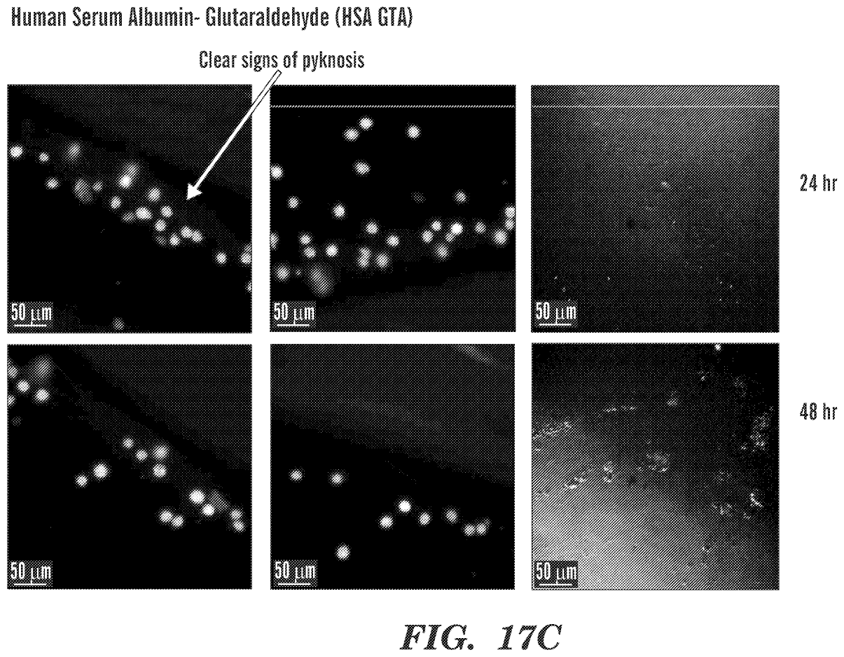

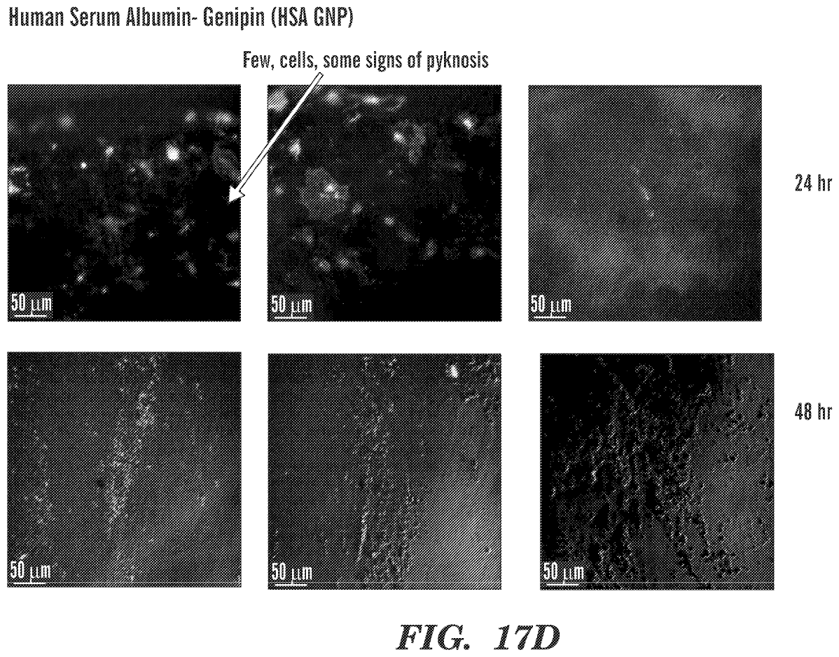

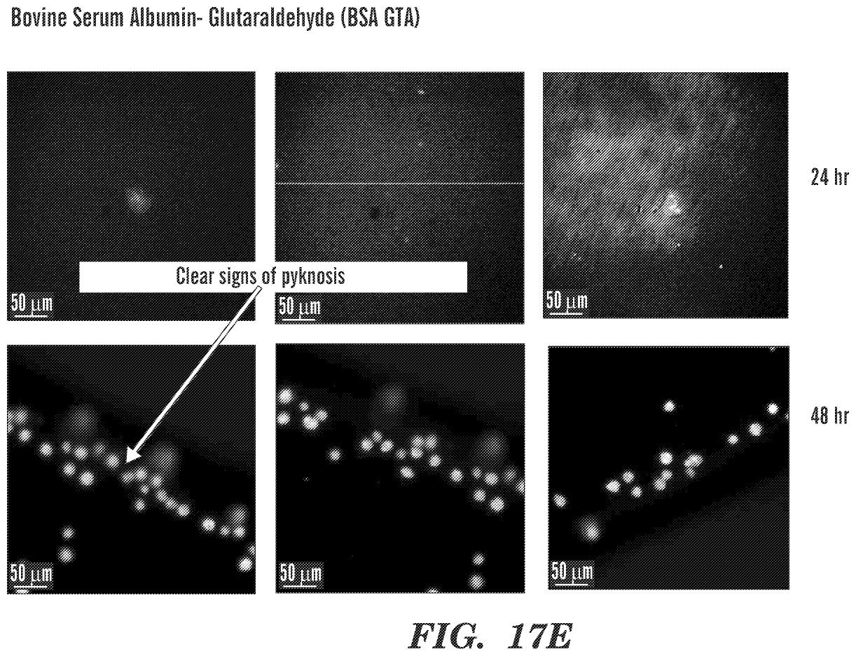

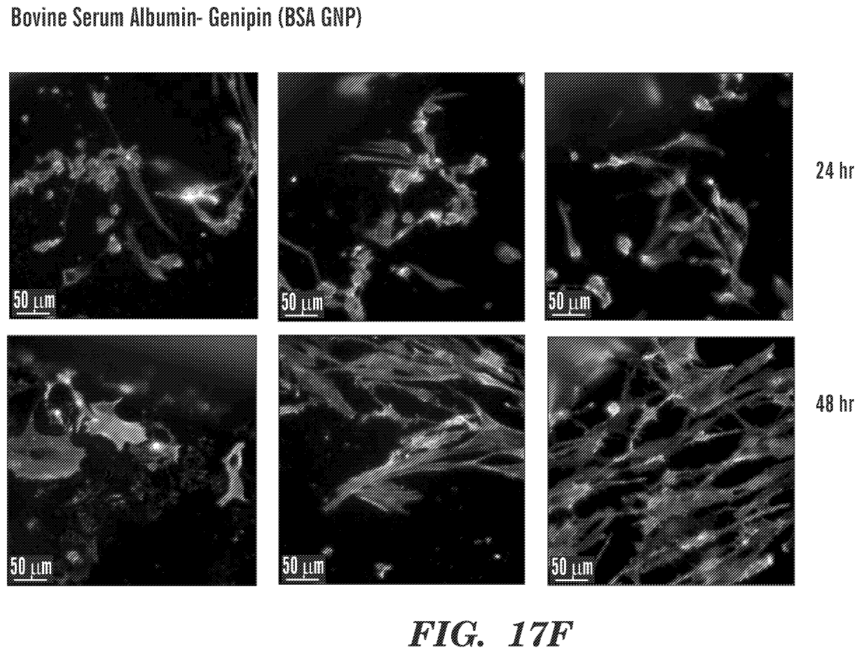

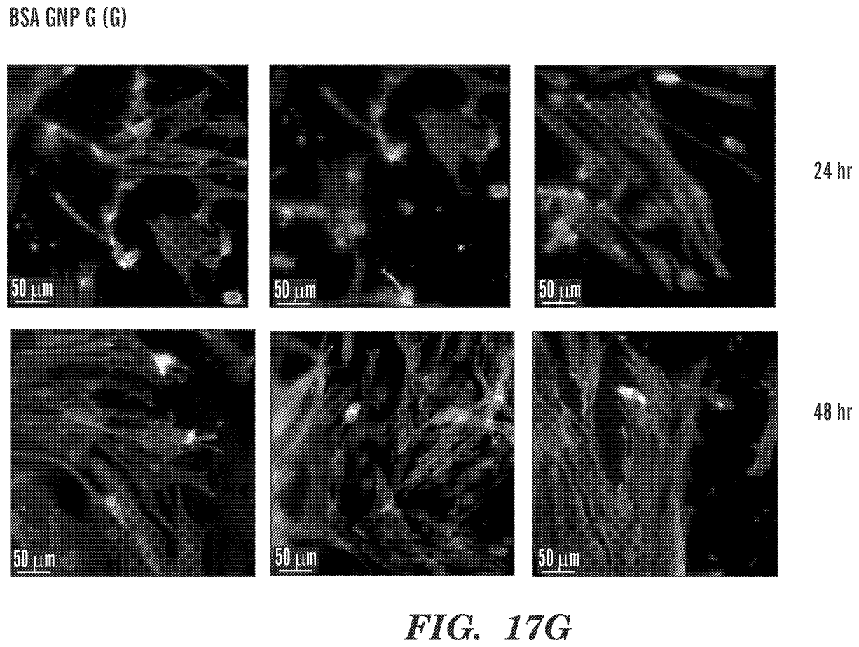

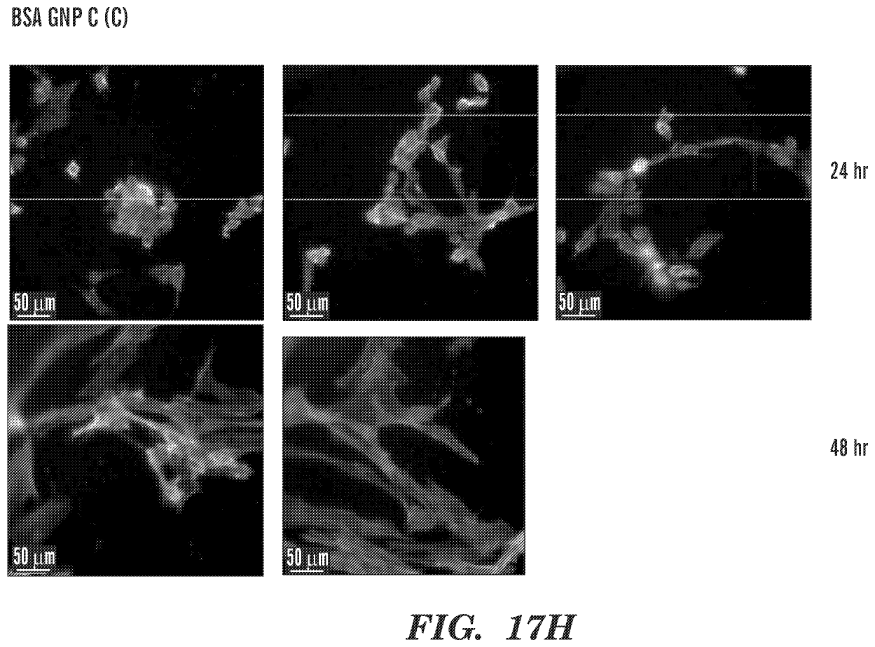

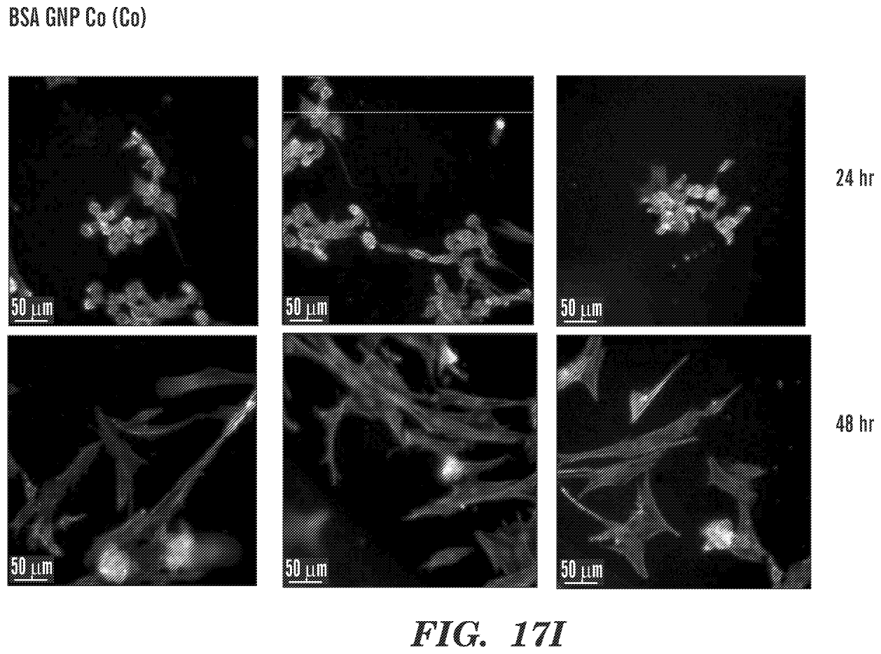

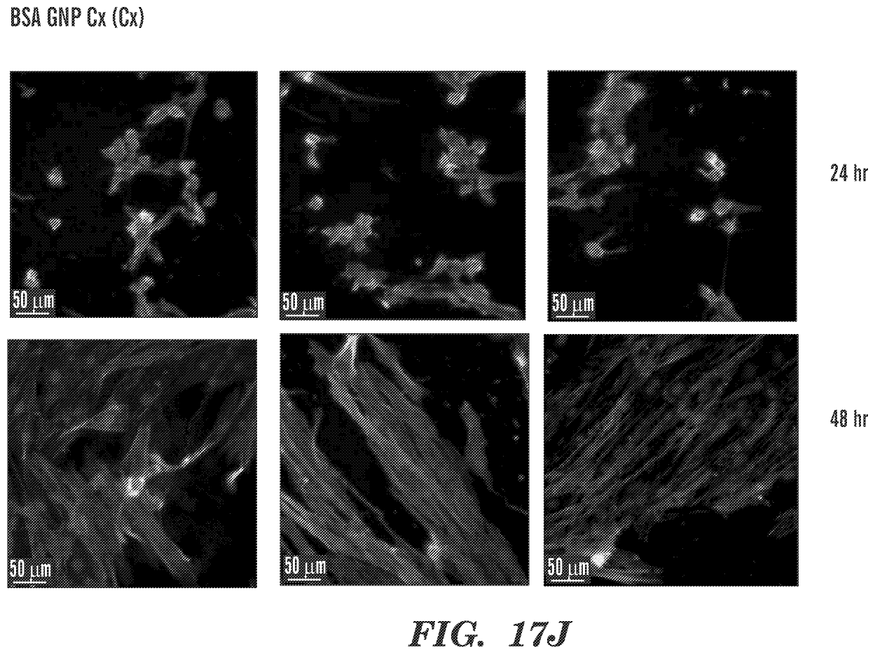

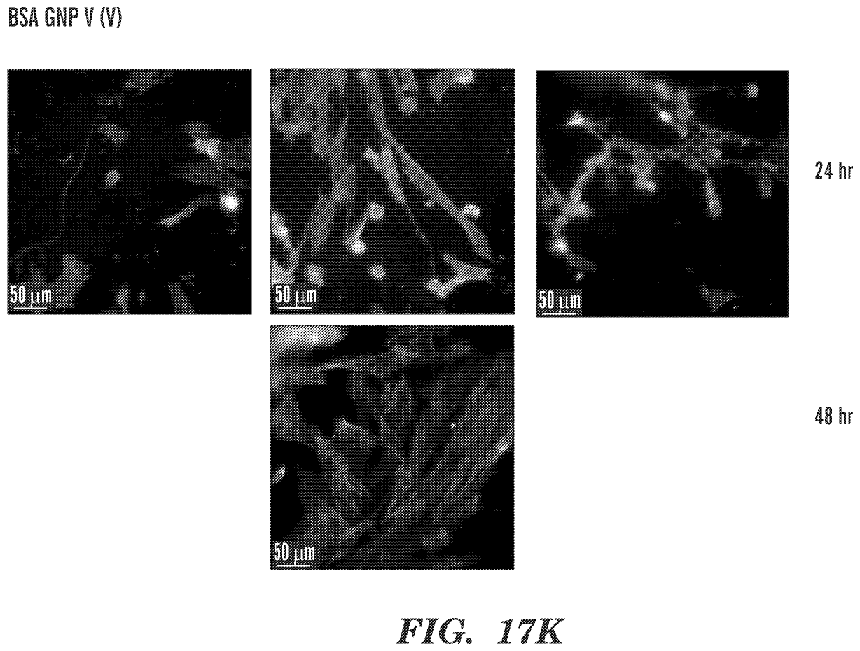

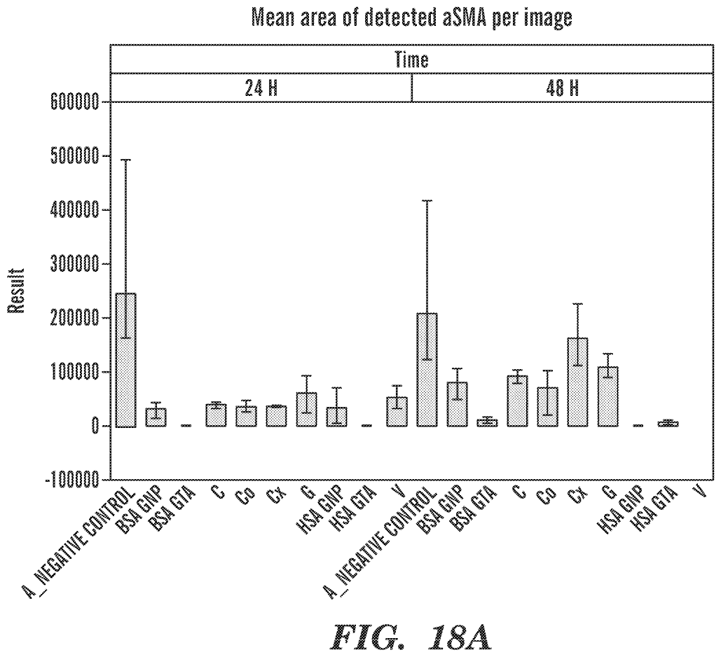

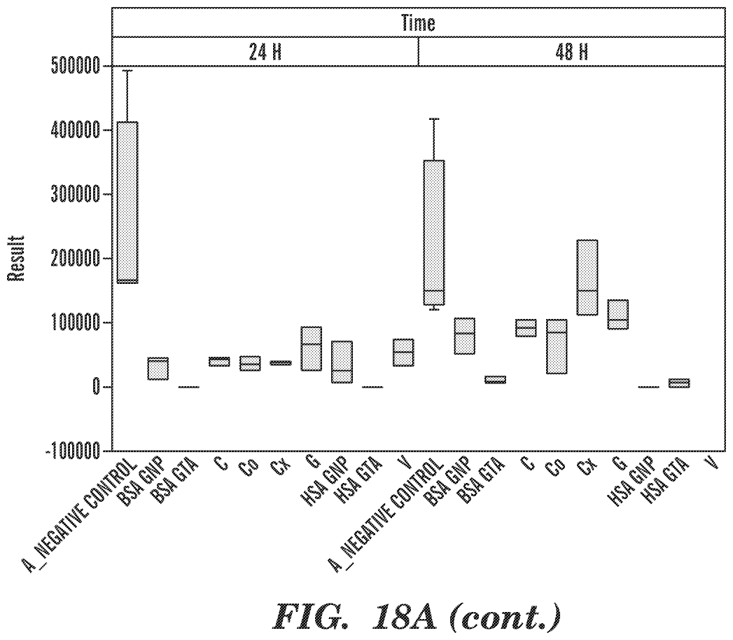

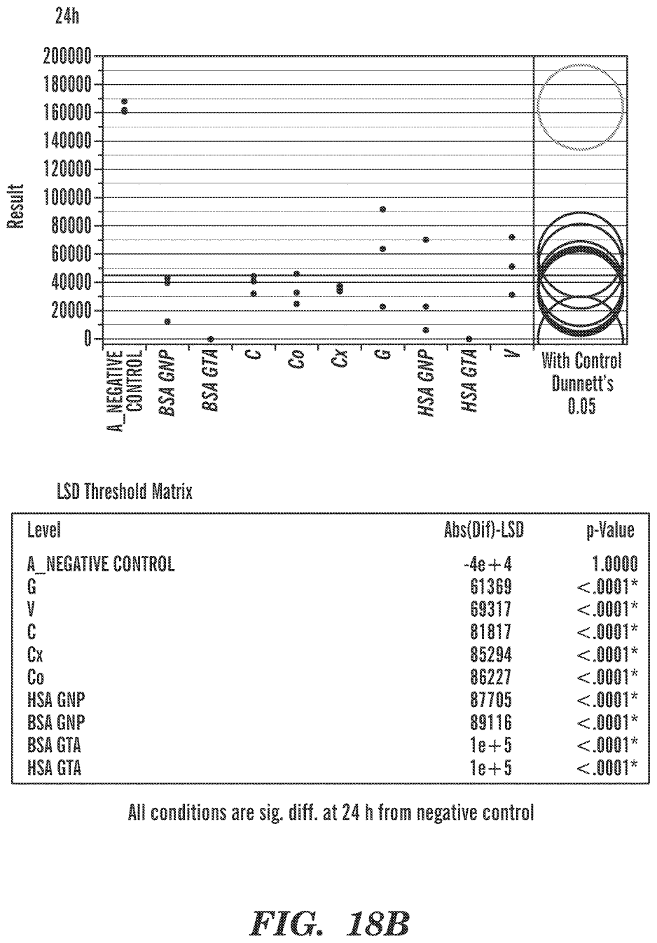

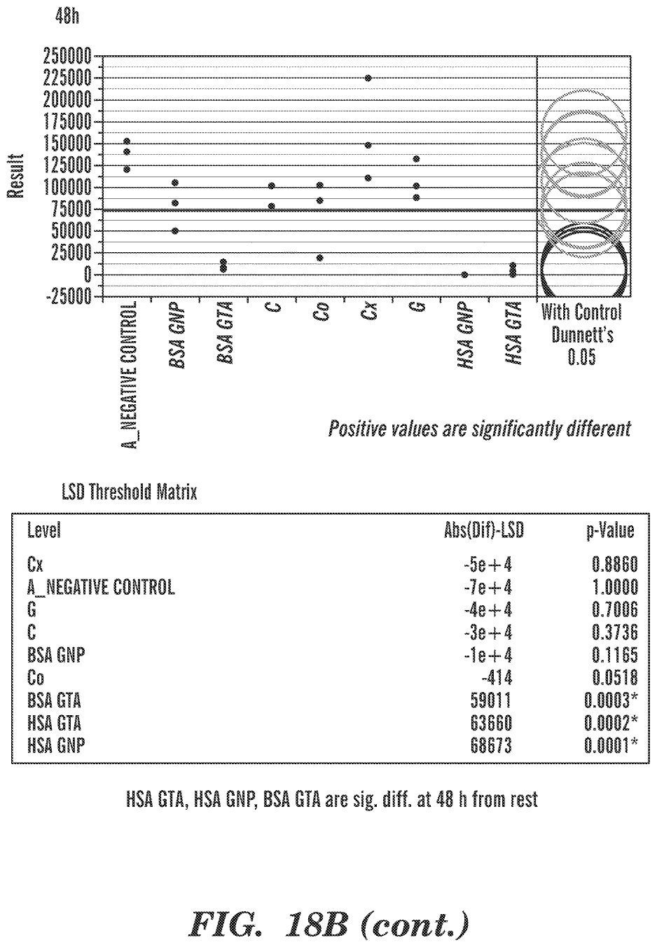

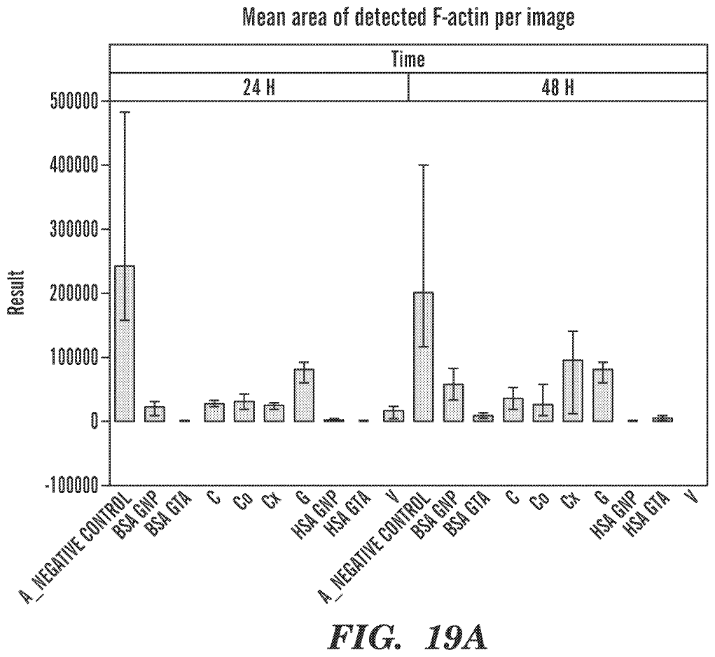

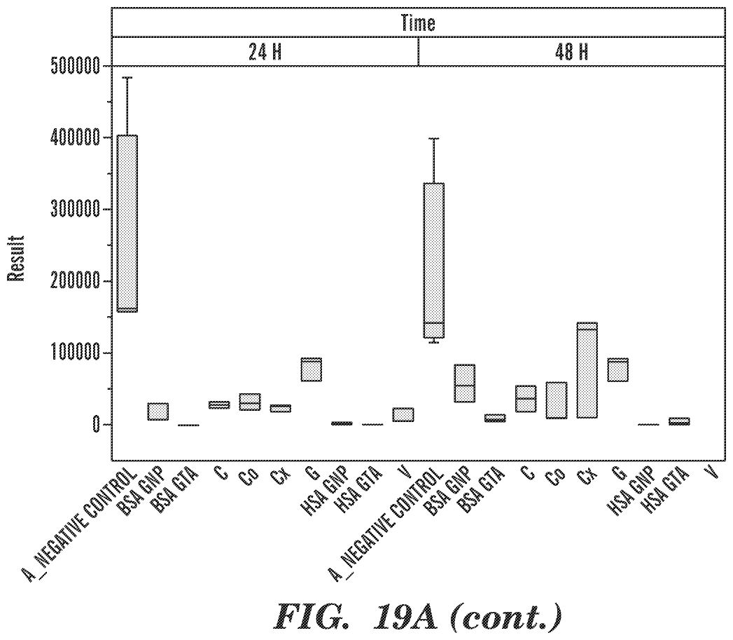

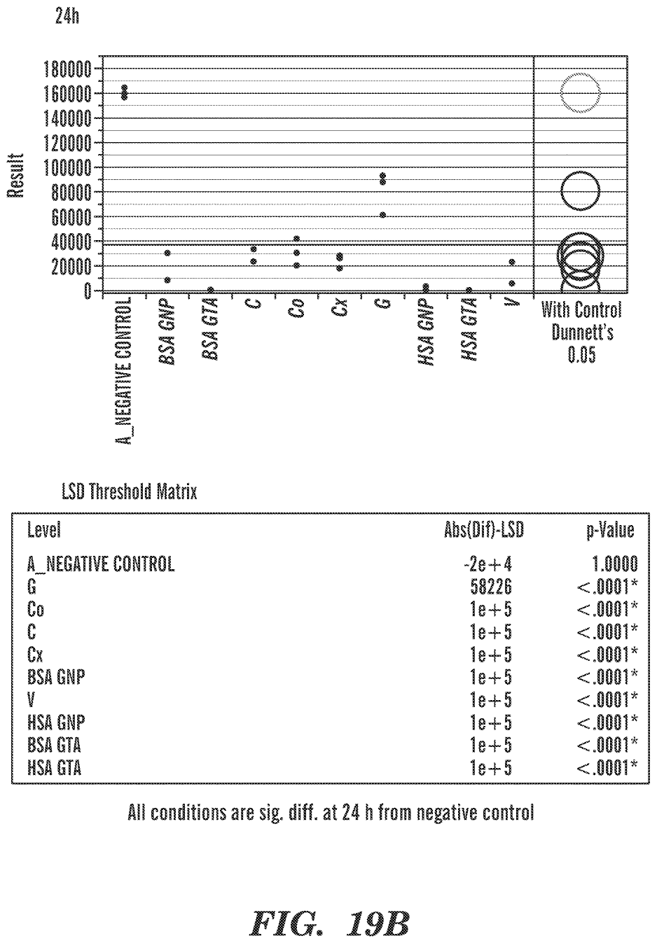

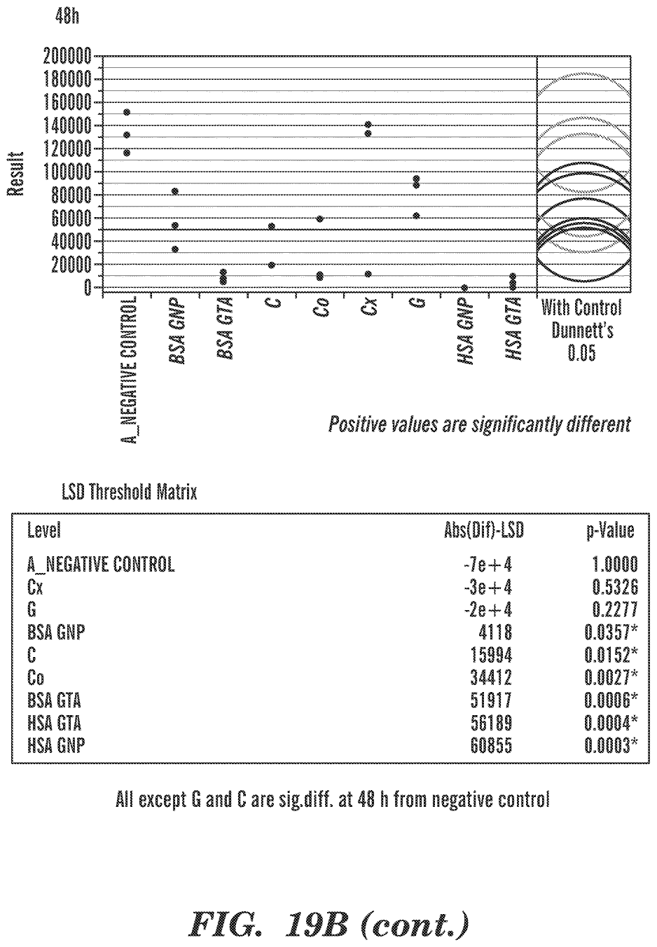

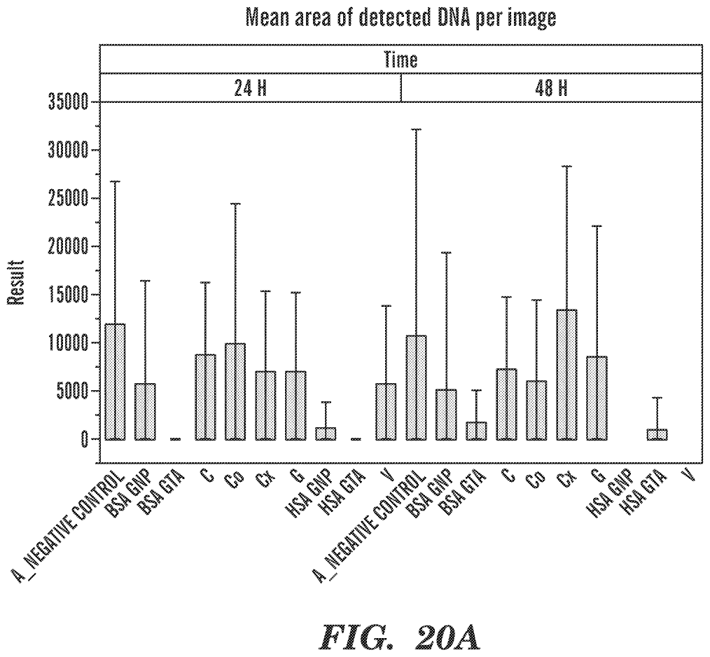

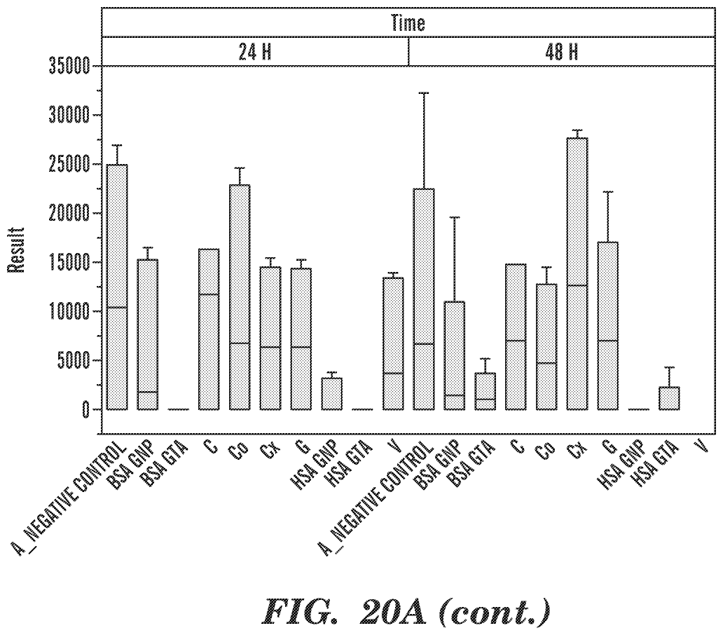

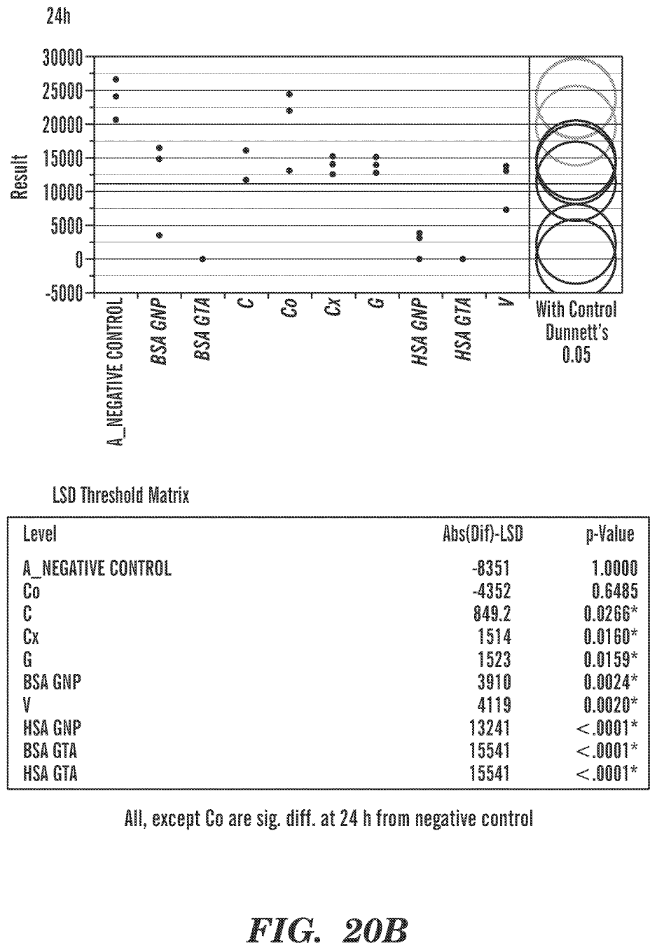

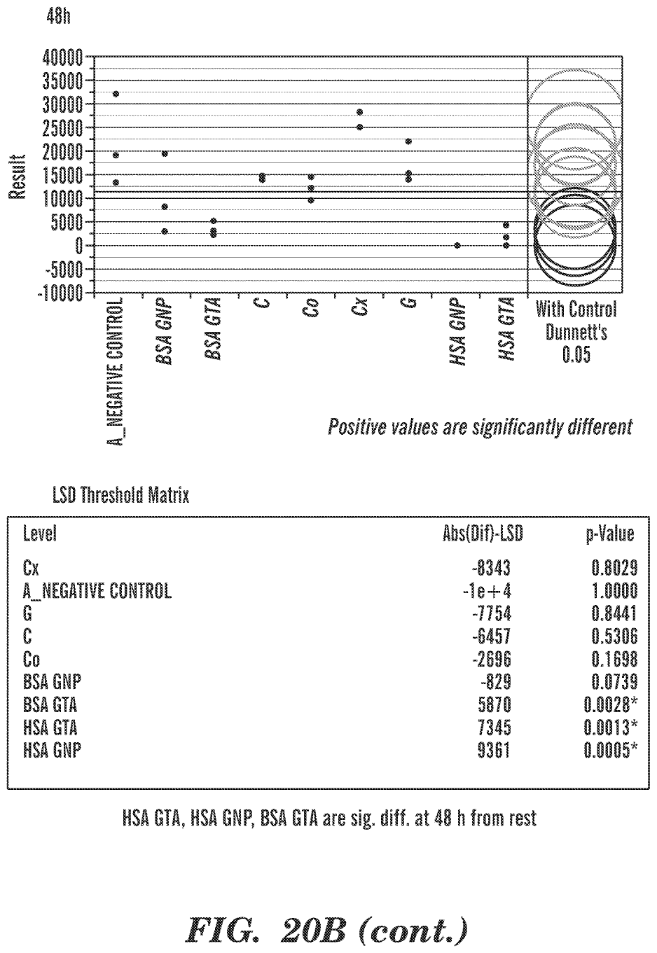

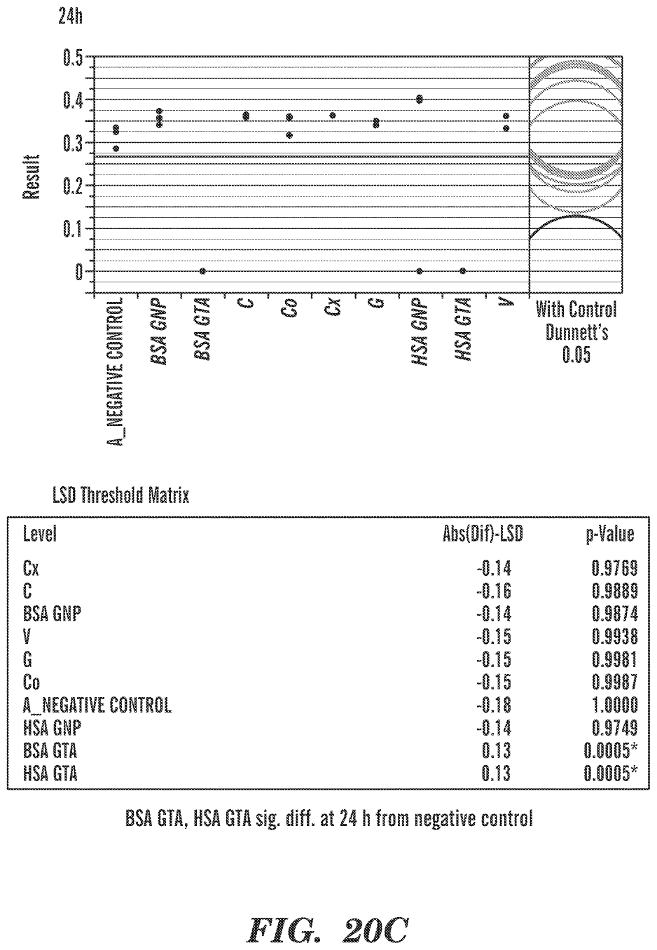

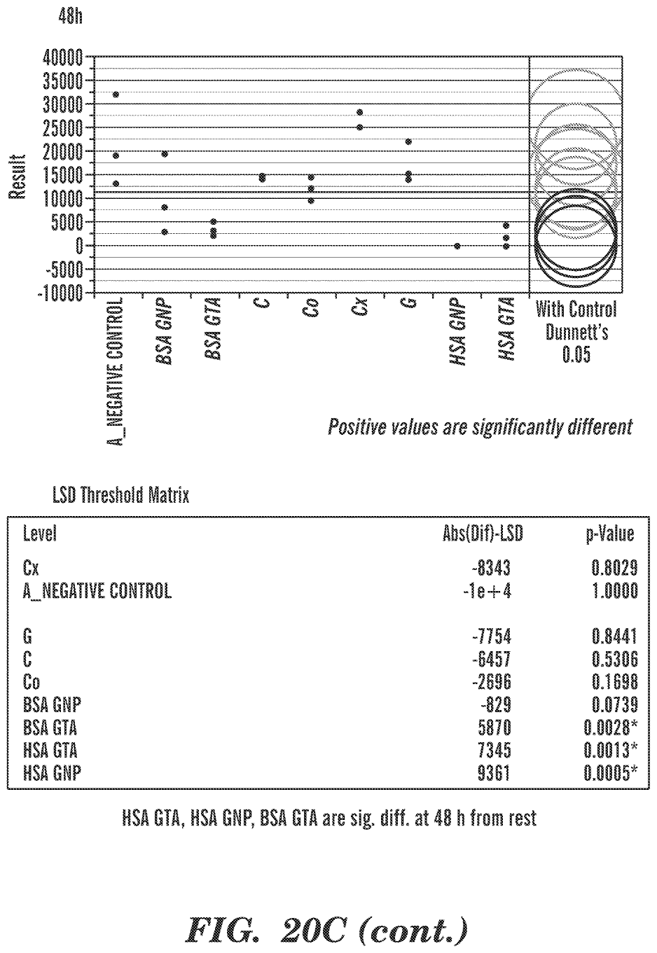

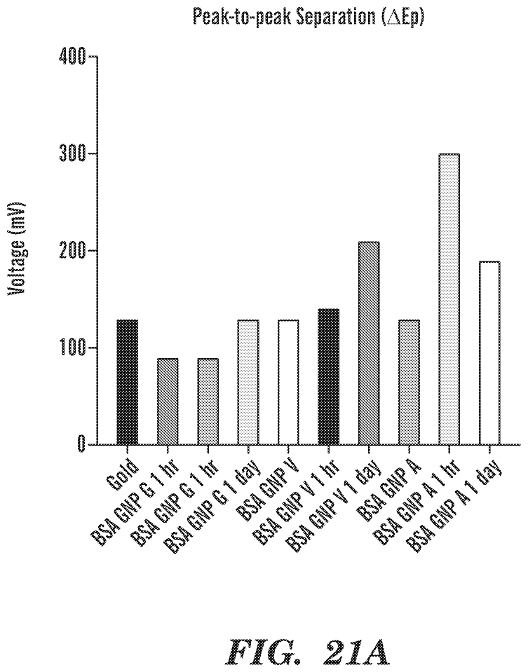

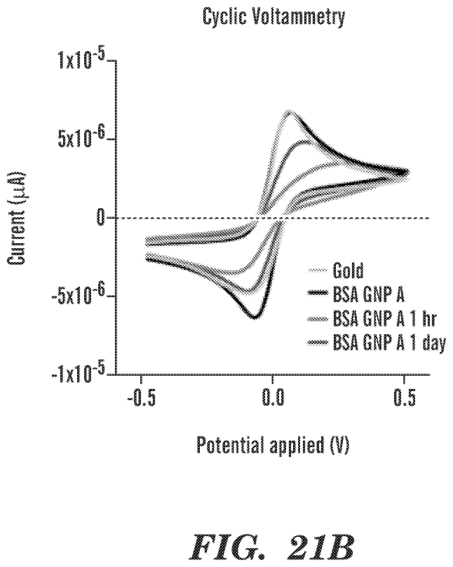

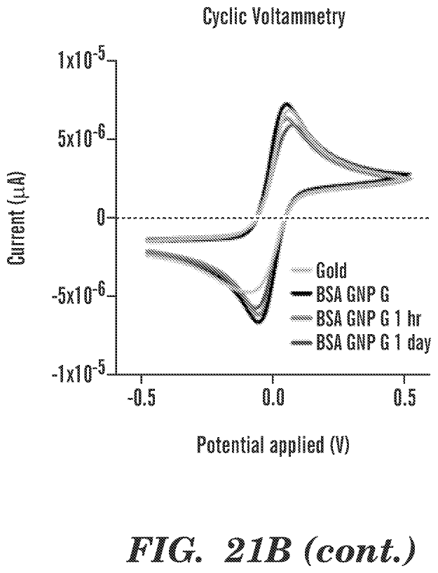

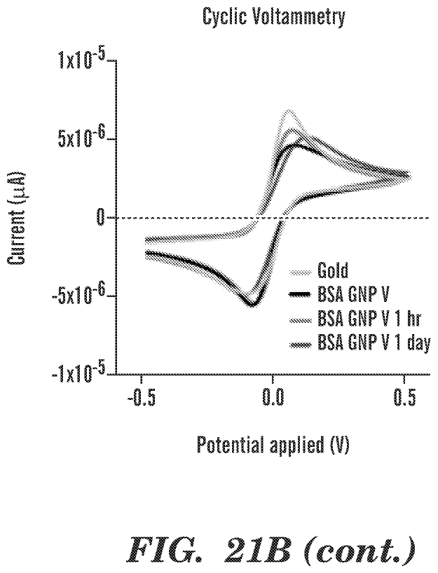

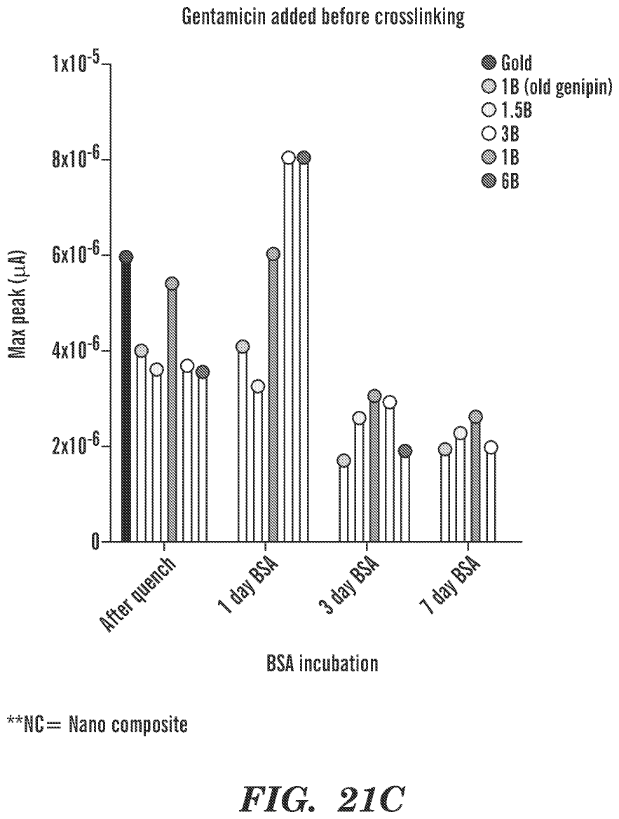

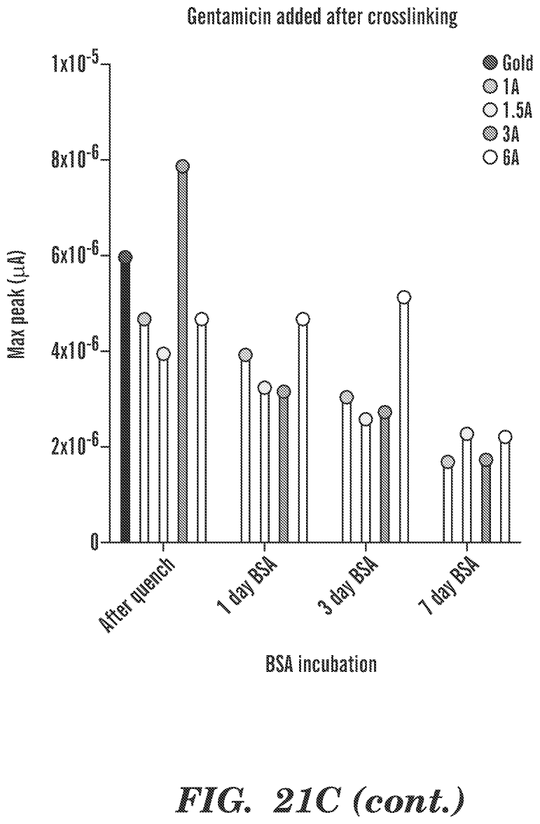

The GNP crosslinked nanocomposite resulted in increased monocyte viability and metabolic activity, indicating lower toxicity than the original GTA-linked coating as seen in FIGS. 12A and 12B.

To assess the viability of the BSA/GOx/GA/antibiotic coatings to perform an electrochemical assay, coated chips were functionalized with capture probes, and an immunoassay was performed. Four groups were created: three groups were coated with a BSA/GOx/GA with vancomycin, gentamicin, or ampicillin. One group was coated without an additional antibiotic component as a control. First, sensors were covalently linked with capture probes (MIP-1β capture antibodies) through 1-Ethyl-3-(3-dimethylaminopropyl)carbodiimide/N-hydroxysuccinimide (EDC/NHS) chemistry. Following this linking, the surface chemistry was quenched using ethanolamine and bovine serum albumin (BSA). To investigate whether this change in surface chemistry caused any sensitivity decrease, three different concentrations of MIP- 16 antigen: 0 (baseline), 0.1, and 10 ng/mL were incubated on separate chips. Chips were then incubated with detection antibody conjugated to biotin, followed by streptavidin-poly(horseradish peroxidase) (HRP), and finally 3,3′,5,5′-Tetramethylbenzidine (TMB) One.

### REFERENCES

1. Russo, M. J., Han, M., Desroches, P. E., Manasa, C. S., Dennaoui, J., Quigley, A.

F., . . . & Silva, S. M. (2021). Antifouling strategies for electrochemical biosensing: mechanisms and performance toward point of care based diagnostic applications. ACS sensors, 6(4), 1482-1507.

2. Arciola, C. R., Campoccia, D., & Montanaro, L. (2018). Implant infections: adhesion, biofilm formation and immune evasion. Nature Reviews Microbiology, 16(7), 397-409.

3. Xu, J., & Lee, H. (2020). Anti-biofouling strategies for long-term continuous use of implantable biosensors. Chemosensors, 8(3), 66.

4. von Woedtke, T., Schlosser, M., Urban, G., Hartmann, V., Jülich, W. D., Abel, P. U., & Wilhelm, L. (2003). The influence of antimicrobial treatments on the cytocompatibility of polyurethane biosensor membranes. Biosensors and Bioelectronics, 19(3), 269-276.

5. Sabaté del Río, J., Henry, O. Y., Jolly, P., & Ingber, D. E. (2019). An antifouling coating that enables affinity-based electrochemical biosensing in complex biological fluids. Nature nanotechnology, 14(12), 1143-1149.

6. Jolly, P., Henry, O.Y.F., Sabaté del Río, J., Ingber, D.E., “Electrically Conductive Antifouling Coating Composition,” WO2019023567, 2019.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A composition comprising a mixture of a proteinaceous material, a conductive element, and an anti-microbial agent.

2. The composition of claim 1, wherein the anti-microbial agent is covalently linked to the proteinaceous material or the conductive element.

3. The composition of claim 1 or 2, wherein the anti-microbial agent is covalently linked to the proteinaceous material or the conductive element via a linker.

4. The composition of claim 3, wherein the linker comprises genipin, polyethylene glycol (PEG), or glutaraldehyde.

5. The composition of claim 3 or 4, wherein a ratio of the anti-microbial agent to the cross-linker is from about 100:1 to about 10:1 (w/w).

6. The composition of any one of claims 1-5, wherein a ratio of the anti-microbial agent to the proteinaceous material is from about 30:1 to about 10:1 (w/w).

7. The composition of any one of claims 1-6, wherein a ratio of the anti-microbial agent to the conductive element is from about 20:1 to about 5:1 (w/w).

8. The composition of any one of claims 1-7, wherein a ratio of the proteinaceous material to the conductive element is from about 10:1 to about 1:1 (w/w).

9. The composition of any one of claims 1-8, wherein the anti-microbial agent is an anti-bacterial agent, anti-fungal agent or anti-viral agent.

10. The composition of any one of claims 1-9, wherein the anti-microbial agent is an anti-bacterial agent.

11. The composition of claim 10, wherein the anti-bacterial agent is selected from the group consisting of macrolides or ketolides such as erythromycin, azithromycin, clarithromycin, and telithromycin; beta-lactams including penicillin, cephalosporin, and carbapenems such as carbapenem, imipenem, and meropenem; monolactams such as penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, cefiriaxone, cefoperazone, ceftazidime, cefixime, cefpodoxime, ceftibuten, cefdinir, cefpirome, cefepime, cefadroxil, ceftriaxone, ceftobiprole and astreonam; quinolones such as nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, ganefloxacin, gemifloxacin and pazufloxacin; antibacterial sulfonamides and antibacterial sulphanilamides, including para-aminobenzoic acid, sulfadiazine, sulfisoxazole, sulfamethoxazole and sulfathalidine; aminoglycosides such as streptomycin, neomycin, kanamycin, paromycin, gentamicin, tobramycin, amikacin, netilmicin, spectinomycin, sisomicin, dibekalin and isepamicin; tetracyclines such as tetracycline, chlortetracycline, demeclocycline, minocycline, oxytetracycline, methacycline, doxycycline; rifamycins such as rifampicin (also called rifampin), rifapentine, rifabutin, bezoxazinorifamycin and rifaximin; lincosamides such as lincomycin and clindamycin; glycopeptides such as vancomycin and teicoplanin; streptogramins such as quinupristin and daflopristin; oxazolidinones such as linezolid; polymyxin, colistin and colymycin; trimethoprim, bacitracin, and phosphonomycin.

12. The composition of any one of claims 1-9, wherein the anti-microbial agent is an anti-fungal agent.

13. The composition of claim 12, wherein the anti-fungal agent is selected from the group consisting of azoles (e.g., barleyconazole, butoconazole, clortrimazole, econazole, fluconazole, isavuconazole, itraconazole, ketoconazole, miconazole, oxyconazole, posaconazole, ravuconazole, saperconazole, sulconazole, tercocnazole, tioconazole, voriconazole, and ciclopirox), polyenes (e.g., natamycin, lucensomycin, nystatin, amphotericin B, etc.), echinocandins (e.g., Cancidas), pradimicins (e.g., beanomicins, nikkomycins, sordarins, allylamines, etc.), Triclosan, Piroctone, fenpropimorph, terbinafine, cyclopyroxolamine, flucitocin, griseofulvin haloprozin, tolnaftate, naphthypine, hydrochloride, morpholine, butenapin, undecylenic acid, propionic acid, and derivatives and analogs thereof.

14. The composition of any one of claims 1-13, wherein the anti-microbial agent comprises a functional group for cross-linking, e.g., an amino (NH₂) group.

15. The composition of any one of claims 1-9, wherein the anti-microbial agent is an antimicrobial peptide or polymer.

16. The composition of any one of claims 1-9, wherein the anti-microbial agent is a metal particle.

17. The composition of claim 16, wherein anti-microbial agent is titanium oxide, copper, or silver nanoparticles.

18. The composition of any one of claims 1-17, wherein the composition comprises at least two (e.g., 3, 4, 5, or more) different anti-microbial agents.

19. The composition of any one of claims 1-18, wherein the proteinaceous material is denatured.

20. The composition of any one of claims 1-19, wherein the proteinaceous material is non-reversibly denatured.

21. The composition of any one of claims 1-20, wherein the proteinaceous material is a globular protein.

22. The composition of any one of claims 1-21, wherein the proteinaceous material is a non-glycosylated protein.

23. The composition of any one of claims 1-22, wherein the proteinaceous material is a serum albumin protein.

24. The composition of any one of claims 1-23, wherein the proteinaceous material is bovine serum albumin (BSA) or human serum albumin (HSA).

25. The composition of any one of claims 1-24, wherein the proteinaceous material is cross-linked with the conductive element.

26. The composition of any one of claims 1-25, wherein the proteinaceous material is cross-linked to the conductive element by a linker.

27. The composition of claim 26, wherein the linker is genipin, polyethylene glycol, or glutaraldehyde.

28. The composition of any one of claims 1-27, wherein the proteinaceous material is cross-linked to itself.

29. The composition of any one of claims 1-28, wherein the proteinaceous material is cross-linked to itself by a linker.

30. The composition of claim 29 wherein the linker is genipin, polyethylene glycol, or glutaraldehyde.

31. The composition of any one of claims 1-30, wherein the conducting material comprises conductive particles, conductive rods, conductive fibers, conductive nano-particles, conductive polymers, conductive nano-flakes, conductive nanotubes, semi-conductive particles, semi-conductive rods, semi-conductive fibers, semi-conductive nano-particles, semi-conductive nano-flakes, semi-conductive nanotubes, or semi-conductive polymers.

32. The composition of any one of claims 1-31, wherein the conducting material is a metal, a metalloid, a conducting polymer, a conducting carbon based material, or any combination thereof.

33. The composition of any one of claims 1-32, wherein the conducting material comprises an allotrope of carbon atoms arranged in a hexagonal lattice.

34. The composition of any one of claims 1-33, wherein the conducting material is graphite, functionalised graphene oxide, reduced graphene oxide, or carbon nano-tubes (CNTs).

35. The composition of claim 34, wherein the carbon nanotubes are carboxylated carbon nanotubes or aminated carbon nanotubes.

36. The composition of claim 34, wherein the reduced graphene oxide is a carboxylated reduced graphene oxide or an aminated reduced graphene oxide.

37. The composition of any one of claims 1-32, wherein the conductive material comprises gold.

38. The composition of any one of claims 1-37, wherein the mixture further comprises a target binding molecule capable of binding with a target molecule.

39. The composition of claim 38, wherein the target binding molecule is covalently linked to the proteinaceous material, or the conductive or semi-conductive material.

40. The composition of claim 38 or 39, wherein the target binding molecule is a receptor, a ligand for a receptor, an antibody, antigen binding fragment of an antibody, an antigen, an enzyme or a nucleic acid.

41. The composition of any one of claims 38-40, wherein the target binding molecule is an antibody, an antigen binding fragment of an antibody or an antigen.

42. The composition of any one of claims 1-41, wherein the composition further comprises a polymer, e.g., a polymer selected from the group consisting of polyethylene glycol (PEG), alginate, polytetrafluoroethylene (PTFE), polyacetylene (PA), polyaniline (PANI), polypyrrole (PPy), polythiophene (PTH), poly(para-phenylene) (PPP), poly(phenylenevinylene) (PPV), and polyfuran (PF).

43. The composition of any one of claims 1-41, wherein the composition further comprises a a therapeutic agent, e.g., anti-inflammatory drugs sirolimus, everolimus, biolimus (A9), zotarolimus (ABT-578), tacrolimus, and pimecrolimus, genistein, steroids (dexamethasone, prednisolone, methylprednisolone and hydrocortisone), fluocinolone acetonide, hormones etc.

44. The composition of any one of claims 1-42, wherein the mixture is a homogenous mixture.

45. The composition of any one of claims 1-43, wherein the composition is antifouling.

46. The composition of any one of claims 1-44, wherein the composition has antimicrobial activity.

47. A surface with antifouling properties, wherein the surface comprises an antifouling coating layer comprising a composition of any one of claims 1-46.

48. The surface of claim 47, wherein the antifouling coating layer is directly or indirectly connected with an electrode.

49. An electrode comprising: (i) a conductive substrate (e.g., an electrically conductive substrate); and (2) an antifouling coating layer on at least a portion of a surface of the conductive substrate, and wherein the antifouling coating layer comprises a composition of any one of claims 1-46.

50. The electrode of claim 49, wherein the conductive substrate comprises gold, silver, copper, platinum, aluminum, stainless steel, tungsten, indium tin oxide, titanium, lead, nickel, palladium, zirconium, niobium, tantalum, chromium, molybdenum, manganese, rhenium, ruthenium, rhodium, iridium, silicon, polyimide, parylene, benzocyclobutene, carbon, graphite, or any combination thereof.

51. The electrode of claim 49 or 50, wherein the conductive substrate comprises a flexible substrate.

52. The electrode of claim 51, wherein the flexible substrate comprises polyethylene terephthalate, polyethylene naphathalate, polyimides, polymeric hydrocarbons, celluloses, plastics, polycarbonates, polystyrenes, or any combination thereof.

53. The electrode of any one of claims 49-52, wherein the antifouling coating layer is adapted for contact with an analyte or a sample comprising an analyte.

54. The electrode of any one of claims 49-53, wherein the electrode is a planar or 3-dimensional electrode.

55. The electrode of any one of claims 49-54, wherein the antifouling coating layer has a thickness of from about 0.1 nm to about 10 μm.

56. An organic field effect transistor comprising:
a. a source electrode;
b. a drain electrode;
c. an organic conducting or semiconducting layer connecting the source electrode to the drain electrode; and
d. an antifouling coating layer on at least a portion of the surface of the organic conducting or semiconducting layer, and wherein the antifouling coating layer comprises a composition of any one of claims 1-46.

57. The organic field effect transistor of claim 56, wherein the organic conducting or semiconducting layer has a thickness of from about 0.1 nm to about 10 μm.

58. The organic field effect transistor of claim 56 or 57, wherein the antifouling coating layer has a thickness of from about 0.1 nm to about 10 μm.

59. The organic field effect transistor of any one of claims 56-58, wherein the organic conducting or semiconducting layer comprises one or more organic compounds having conducting and/or semiconducting properties.

60. The organic field effect transistor of claim 59, wherein the organic compounds having conducting and/or semiconducting properties are selected from the group consisting of polyacetylenes, porphyrins, phthalocyanins, fullerenes, polyparaphenylenes, polyphenylenevinylenes, polyfluorenes, polythiophenes, polypyrroles, polypyridines, polycarbazoles, polypyridinevinylenes, polyarylvinylenes, poly (p-phenylmethylvinylenes), derivatives and co-polymers thereof, and any combination thereof.

61. The organic field effect transistor of claim 59 or 60, wherein the organic compounds having conducting and/or semiconducting properties are selected from the group consisting of poly(3-hexylthiophene) (P3HT), perfluoro-3,6-dioxa-4-methyl-7-octenesulfonic acid copolymer, tetrafluoroethylene-perfluoro-3,6-dioxa-4-methyl-7-octenesulfonic acid copolymer (e.g. nafion), poly(9,9-dioctylfluorene-2,7-diyl-co-bis-N, N-(4-butylphenyl)-bis-N, N-phenyl-1,4-phenylenediamine), poly(9,9-dioctylfluorene-2,7-diyl-co-benzothiadiazole), (6,6)-phenyl-C61-butyric acid methyl ester, poly(2-methoxy-5-(2′-ethyl-hexyloxy)-1,4-phenylene vinylene), poly(4-vinylphenol) (PVP), and copolymers thereof, and any combination thereof.

62. The organic field effect transistor of any one of claims 56-61, wherein the organic field effect transistor further comprises a conducting polymer layer between the organic conducting or semiconducting layer and the antifouling layer.

63. The organic field effect transistor of claim 62, wherein the conducting polymer layer comprises one or more of fluorinated polymer, poly methyl methacrylate (PMMA), polyhydroxyethylmethacrylate (PolyHEMA), polyacetylenes, porphyrins, phthalocyanins, fullerenes, polyparaphenylenes, polyphenylenevinylenes, polyfluorenes, polythiophenes, polypyrroles, polypyridines, polycarbazoles, polypyridinevinylenes, polyarylvinylenes, poly(p-phenylmethylvinylenes), and derivatives, co-polymers, mixtures and blends thereof.

64. The organic field effect transistor of claim 62 or 63, wherein the conducting polymer layer comprises one or more of poly(3-hexylthiophene) (P3HT), perfluoro-3,6-dioxa-4-methyl-7-octenesulfonic acid copolymer, tetrafluoroethylene-perfluoro-3,6-dioxa-4-methyl-7-octenesulfonic acid copolymer (e.g. nafion), poly(9,9-dioctylfluorene-2,7-diyl-co-bis-N, N-(4-butylphenyl)-bis-N, N-phenyl-1,4-phenylenediamine), poly(9,9-dioctylfluorene-2,7-diyl-co-benzothiadiazole), (6,6)-phenyl-C61-butyric acid methyl ester, poly(2-methoxy-5-(2′-ethyl-hexyloxy)-1,4-phenylene vinylene), poly(4-vinylphenol) (PVP), and copolymers thereof, and any combination thereof.

65. The organic field effect transistor of any one of claims 62-64, wherein the conducting polymer layer has a thickness of from about 0.1 nm to about 10 μm.

66. The organic field effect transistor of any one of claims 56-65, wherein the organic field effect transistor further comprises gate electrode in contact with the antifouling coating layer or the organic conducting or semiconducting layer.

67. The organic field effect transistor of any one of claims 56-66, wherein the antifouling coating layer and/or the organic conducting or semiconducting layer is configured to be connected to an ohmic conductor for applying a gate voltage to said organic conducting or semiconducting layer (e.g., via antifouling layer).

68. The organic field effect transistor of claim 67, wherein the antifouling coating layer is configured to be connected to the ohmic conductor.

69. The organic field effect transistor of claim 67 or 68, wherein the organic field effect transistor further comprises an ohmic conductor in contact with the antifouling coating layer and/or the organic conducting or semiconducting layer.

70. The organic field effect transistor of any one of claims 67-69, wherein the ohmic conductor is a gate electrode.

71. A method for detecting a target analyte in a sample, the method comprising: contacting a sample suspected of comprising a target analyte with an electrode of any one the preceding claims and detecting binding of the target analyte with the target binding ligand.

72. The method of claim 71, wherein said detecting the binding of the target molecule with the target binding ligand comprises applying a voltage to the electrode.

73. The method of claim 71 or 72, wherein said detecting the binding of the target molecule with the target binding ligand comprises measuring a current generated from electrode.

74. A method for detecting a target analyte in a sample, the method comprising: contacting a sample suspected of comprising a target analyte with an organic field effect transistor of any one of the preceding claims and detecting binding of the target analyte with the target binding ligand.

75. The method of claim 74, wherein said detecting the binding of the target molecule with the target binding ligand comprises applying a voltage to the drain electrode.

76. The method of claim 74 or 75, wherein said detecting the binding of the target molecule with the target binding ligand comprises applying a voltage to the ohmic conductor.

77. The method of any one of claims 74-76, wherein said detecting the binding of the target molecule with the target binding ligand comprises grounding the source electrode.

78. The method of any one of claims 74-77, wherein said detecting the binding of the target molecule with the target binding ligand comprises detecting a drain current.

79. The method of any one of claims 71-78, wherein said detecting the binding of the target molecule with the target binding molecule comprises contacting a second target binding molecule to the target molecule, wherein the second target binding molecule comprises a detectable label.

80. The method of claim 79, wherein said contacting with the second target binding molecule is prior to contacting the sample with the electrode or the organic field effect transistor.

81. The method of claim 79, wherein said contacting with the second target binding molecule is after contacting the sample with the electrode or the organic field effect transistor.

82. The method of any one of claims 79-81, wherein the detectable label comprises an enzyme, a fluorophore, a chemiluminescent label, colloidal gold, colored glass or plastic (e.g., polystyrene, polypropylene, and latex) beads, a radiolabel, a quantum dot, or any combination thereof.

83. The method of any one of claims 79-82, wherein the detectable label comprises an enzyme.

84. The method of claim 83, wherein the enzyme is a peroxidase, alkaline phosphatase, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase or acetylcholinesterase.

85. The method of claim 83 or 84, wherein the enzyme is a peroxidase or alkaline phosphatase.

86. The method of any one of claims 83-85, wherein the method further comprises contacting the enzyme with a substrate of the enzyme.

87. The method of any one of claims 79-86, wherein the detectable label facilitates generation of a charge carrier.

88. The method of claim 87, wherein said detecting the binding of the target molecule with the target binding molecule comprises detecting the charge carrier.

89. The method of any one of claims 79-88, wherein the target analyte is a peptide, a polypeptide, a peptidomimetic, a nucleic acid, an oligosaccharide, a polysaccharide, an amino acid, nucleoside, a nucleotide, a carbohydrate, a lipid, a peptidoglycan, a cell, microbial matter, an antigen, a lipid, a steroid, a hormone, a lipopolysaccharide, an endotoxin, a therapeutic agent, a lipid-binding molecule, a cofactor, a small molecule, a toxin, a biological threat agent (e.g., spore, viral, cellular and protein toxin), or any combination thereof.

90. The method of any one of claims 79-89, wherein the target analyte is a protein, an antibody, an antigen binding fragment of an antibody, an antigen, a hormone, or a metabolite.

91. The method of any one of claims 79-90, wherein the target analyte is a tumour marker or a clinical chemistry target.

92. The method of any one of claims 79-91, wherein the sample is a biological sample (e.g., blood, saliva, amniotic fluid, sputum, urine, semen, cerebrospinal fluid, bronchial aspirate, perspiration, mucus, liquefied stool sample, synovial fluid, lymphatic fluid, tears, tracheal aspirate, lactation product, and any combination thereof).

93. The method of any one of claims 79-92, wherein the sample is a food, an ingredient for preparing a food, poultry, meat, fish, beverage, or dairy product.

94. The method of any one of claims 79-93, wherein the sample is a non-biological sample (e.g., water, salt water, pond water, river water, reservoir water, brine, drinking water, industrial water, brown water, waste water, sewerage, soil, and mixtures thereof.

95. The method of any one of claims 79-94, wherein the sample is pre-processed prior to contacting with the electrode or the organic field effect transistor.

96. A sensor comprising an electrode of any one of claims 49-55 or an organic field effect transistor of any one claims 56-70.

97. The sensor of claim 96, wherein the sensor comprises a fluid-contact surface and the electrode or the organic field effect transistor is immobilized on at least a portion of the fluid-contact surface.

98. The sensor of claim 96 or 97, wherein the sensor comprises one or more microfluidic flow cells.

99. The sensor of any one of claims 96-98, wherein the sensor comprises one or more microfluidic flow cells.

100. The sensor of any one of claims 97-99, wherein the fluid-contact surface further comprises a positive control electrode and/or a negative control electrode immobilized thereon.

101. Use of an electrode of any one of claims 49-55 or an organic field effect transistor of any one claims 56-70 for detecting a target analyte in a sample.

102. A kit comprising a composition, surface, electrode, organic field effect transistor or sensor of any one of the preceding claims.
